SEQUENCE LISTING (27 sequences)

SEQ ID NO: 1 - DNA, 737 bp, Nicotiana tabacum
  source 1..737: mol_type = other DNA
atggtactaaaaacaatagaaattttgaagaatgaaattgccatagaggaggaatcagtgattatcactgaagatgttaaggccggtcttgttcttgtggacatcatcaatggcttttgcaccgttggggctggaaatctggcaccaagagagccaaacaggcagatctctgaaatgattgatgaatcaacaaggctggctagattgttctgtgacaagaaatggcctattcttgcctttcttgattcacaccaccctgacaaacttgaacacccttatcctcctcactgtatcactggcactgatgaatccaatttggttcctgcactaagatggttggagaaggaacaaaacgtaacaatcaggcgtaaagactgctatgatggctatattggttcgtttcaggaggatggctctaatgtatttgttgattgggtgaaaaacaacaaaattcaaactttgttggttgtaggggcatgtacagacatttgtgtgctggatttcgtttgctctacattatcagctaggaacccggcttcctcgaccctttggaggaagtagtagtttactctcagggatgtgccacttttgattttcctgcctccatggcaagaaacaccaaagatatttcaccacatcctcaggagctgatgcatcatgtaggactttatatggcgaaggaaaggggtgctaaaatagctagagaagtctccttcaacagcttgaagaaaccatga

SEQ ID NO: 2 - protein, 244 aa, Nicotiana tabacum
MVLKTIEILKNEIAIEEESVIITEDVKAGLVLVDIINGFCTVGAGNLAPREPNRQISEMIDESTRLARLFCDKKWPILAFLDSHHPDKLEHPYPPHCITGTDESNLVPALRWLEKEQNVTIRRKDCYDGYIGSFQEDGSNVFVDWVKNNKIQTLLVVGACTDICVLDFVCSTLSARNRGFLDPLEEVVVYSQGCATFDFPASMARNKDISPHPQELMHHVGLYMAKERGAKIAREVSFNSLKKP

SEQ ID NO: 3 - DNA, 738 bp, Nicotiana tabacum
  source 1..738: mol_type = other DNA
atggtaataaaaacaatagatattttgaagaatgaaattcccatagaggaggaatcactgatcatcactgaagatgtgaaagctggtcttgttcttgtggatatcatcaatggcttttgcactgttggagctggaaatctggcaccaagagagccaaacaggcagatctctgaaatgattgaggaatcaaaaaggctggctagattgttctgtgacaagaaatggcctattcttgcctttcttgattcacaccaacctcacaaacttgaacacccttatcctcctcactgtatcactggcactgatgaatccaatttggttcctgcactaagatggttggagaaggaacaaaacgtaacaatcaggcgtaaagattgctacgatggctatattggttcttttcaggaggatggctctaatgtatttgttgattgggtgaaaaacaacaaaattcaaactttgttggttgtaggggtatgtacagacatttgtgtgctggatttcgtttgctctacattatcagctaagaaccgcggtttcctcaatcctttggaacaagtagtagtttactctaaaggatgtgccacttttgattttccagcctccatggcaagaaacagcaaagatatttcaccacatcctcaggagctgatgcatcatgtaggactttatatggcgaaggaaaggggtgctaaaatagctagagaagtctccttcgacagcttgaagaaaccatga

SEQ ID NO: 4 - protein, 245 aa, Nicotiana tabacum
MVIKTIDILKNEIPIEEESLIITEDVKAGLVLVDIINGFCTVGAGNLAPREPNRQISEMIEESKRLARLFCDKKWPILAFLDSHQPHKLEHPYPPHCITGTDESNLVPALRWLEKEQNVTIRRKDCYDGYIGSFQEDGSNVFVDWVKNNKIQTLLVVGVCTDICVLDFVCSTLSAKNRGFLNPLEQVVVYSKGCATFDFPASMARNSKDISPHPQELMHHVGLYMAKERGAKIAREVSFDSLKKP

SEQ ID NO: 5 - DNA, 753 bp, Nicotiana tabacum
  source 1..753: mol_type = other DNA
atgggaacggtggcgaaagaggcggcgattgatttactgaaaaatgagattccggtgaaggaagatgagcctctgtttctctccggcgacgtcaacaccggtctcgtactcgtcgacatcgttaatggcttctgcactgtcggcgctggctatttggctccagtagcacctgataaacaaatttcagcaatggttgatgagtcggttaaacttgcaaaattgttttgtgagaagaaatggcctatttatgctcttcttgattctcatcatcctaatgtgcccgagccacctcatcctcctcattgtatcgctggaacggatgaatctaagttggttcctgctctgcagtggttggaaagtgaaccgaatgtgacactgcgatgcaaggattgcattgatgggttcgttggttcgattgagaaagatggatctaatgtctttgtagattgggtgaaagctaatgagattaaagctatattggttgtagggatatgcacggacatttgcctgcttgattttgtgtgttctgccttatctgcaaggaaccgtggatttctctcccccctggaagatgtgattgtatattcccgtggctgttctacttttgatcttccagcacagattgctaacataaaaggagccttacctcatccacaggaattgatgcatcatataggcctttacatggccaaaggaagaggagcaaaagtagtttcagaggtctcctttgatacatatgcaaaagcgacataa

SEQ ID NO: 6 - protein, 250 aa, Nicotiana tabacum
MGTVAKEAAIDLLKNEIPVKEDEPLFLSGDVNTGLVLVDIVNGFCTVGAGYLAPVAPDKQISAMVDESVKLAKLFCEKKWPIYALLDSHHPNVPEPPHPPHCIAGTDESKLVPALQWLESEPNVTLRCKDCIDGFVGSIEKDGSNVFVDWVKANEIKAILVVGICTDICLLDFVCSALSARNRGFLSPLEDVIVYSRGCSTFDLPAQIANIKGALPHPQELMHHIGLYMAKGRGAKVVSEVSFDTYAKAT

SEQ ID NO: 7 - DNA, 753 bp, Nicotiana tabacum
  source 1..753: mol_type = other DNA
atgggaacggtggcgaaagaggcggcgattgatttactgaaaaatgagattccggtggaggaagacgagcctctgtttatctccggcgacgtcaacaccggtctcgtactcgtggacatcgttaatggcttctgcactgtcggcgctggcaatttggctccagtggcacccgataaacaaatttcagcaatggttgatgagtcggttaaacttgcaaaaatgttttgtgagaagaaatggcctatttatgctcttcttgattctcatcatcctgatgtgcctgagccacctcatcctcctcattgtatcgctggaacagatgaatctaagttggttcctgctctgcaatggttggaaagtgaaccaaatgtgacactgcgatgcaaggattgcattgatgggtttcttggttcgattgagaaggatggatctaatgtcttcgtagattgggtgaaagctaatgagattaaagctatattggttgtagggatatgcacggacatgtgcgtgcttgattttgtgtgttctgccttatctgcaaggaaccgtggatttctctcccccctggaagatgtgattgtatattcccgtggctgtgctacttttgatcttccactgcagattgctaacataaaaggagccttacctcatccacaagaattgatgcatcatataggcctttacatggccaaaggaagaggagcaaaagtagtttcagagatctccattgatacaactgcaagagagacataa

SEQ ID NO: 8 - protein, 250 aa, Nicotiana tabacum
MGTVAKEAAIDLLKNEIPVEEDEPLFISGDVNTGLVLVDIVNGFCTVGAGNLAPVAPDKQISAMVDESVKLAKMFCEKKWPIYALLDSHHPDVPEPPHPPHCIAGTDESKLVPALQWLESEPNVTLRCKDCIDGFLGSIEKDGSNVFVDWVKANEIKAILVVGICTDMCVLDFVCSALSARNRGFLSPLEDVIVYSRGCATFDLPLQIANIKGALPHPQELMHHIGLYMAKGRGAKVVSEISIDTTARET

SEQ ID NO: 9 - DNA, 591 bp, Nicotiana tabacum
  source 1..591: mol_type = other DNA
atggcttcctcatcatacaggaaatatgaaacccgaaagcgaaatccagacccgaaatctgcggttcttttagtgatagacatacagaaccacttctactccatggccaagcctattctccccgctatcaacaccaccatcgacctctgccgacaggcttccgtgcctgtgatattcacgcgccaccgtcacaagtcaccggatgactacggcatgctctacgagtggtggaacggagacgtcatcagagatggcaccattgaagctgaactcataccggagttgaaccggagagatgctgacttggtggtagaaaagcacacttatagtgccttcagagatacaagcctagaggagaaactgttggagatggggataacggaggtaatagtgagtggagtaatgaccaacttgtgttgtgagactacagccagagaggcctttgtgagagggttcagagtcttcttttccacagatgctactgctacttcctcagcagaattgcatgatgctacactgaagaacttagcttatggtttcacttatttggttgactgcaaaaggattcaagatgcgttttcaaattcctga

SEQ ID NO: 10 - protein, 196 aa, Nicotiana tabacum
MASSSYRKYETRKRNPDPKSAVLLVIDIQNHFYSMAKPILPAINTTIDLCRQASVPVIFTRHRHKSPDDYGMLYEWWNGDVIRDGTIEAELIPELNRRDADLVVEKHTYSAFRDTSLEEKLLEMGITEVIVSGVMTNLCCETTAREAFVRGFRVFFSTDATATSSAELHDATLKNLAYGFTYLVDCKRIQDAFSNS

SEQ ID NO: 11 - DNA, 579 bp, Nicotiana tabacum
  source 1..579: mol_type = other DNA
atggcttcctcatcatacaggaaatatgaaacccgaaagcgaaatccagacccgaaatctgcggttcttttagtgatagatatgcagaactacttctactccatggccaagcctattctccccgctatcaacactaccatcgacctctgccgacaggcttccgtgcctgtgatattcacgcgccaccgtcacaagtcaccggatgactacggcatgctctacgagtggtggaacggagacatcattaaagacggcaccgttgaggctgaactcataccggagttggaccgaagagaaagcgacttggtggtggaaaaacacacttacaatacaaacctagaggagaaactgttggagatggggaaaaaggaggtaatagtgagtggagtaatgaccaacctgtgttgtgagacaacagccagagaggcatttgttagaggattcagagtcttcttttcgacagatgctactgctacttcctcagcagaattgcatgatgctacactgaagaacttagcatatggtttcacttatttggttgactgcaaaaggattcaatctgcgttttcaaattcctga

SEQ ID NO: 12 - protein, 192 aa, Nicotiana tabacum
MASSSYRKYETRKRNPDPKSAVLLVIDMQNYFYSMAKPILPAINTTIDLCRQASVPVIFTRHRHKSPDDYGMLYEWWNGDIIKDGTVEAELIPELDRRESDLVVEKHTYNTNLEEKLLEMGKKEVIVSGVMTNLCCETTAREAFVRGFRVFFSTDATATSSAELHDATLKNLAYGFTYLVDCKRIQSAFSNS

SEQ ID NO: 13 - DNA, 585 bp, Nicotiana tabacum
  source 1..585: mol_type = other DNA
atggcctcggaattaaatgttggcaaatggaagaacgtagctctccttgtcatagacatgcagaaagacttcatatttggccctatgcaagtaaaaggaggtcaagctatcgtccctaacgttatcaaagctgttgaggttgcaagaaactgtggcattcacatcgtttgggttgtacgtgagaatgatccattagggagagatgttgaattatttcgtcgacactttaaaccaaagctggcatcaaagggtagtgttggggctgaactagttgatgggctcgttatggacgtagaagaggattacaagctggtcaaaacacgtttcagcgcatttcttaacactcaccttcactcatatcttcagaccaatggcattactgatttggtcttcactggtgtccaaacaccaaattgcatacggcaaactgtatttgatgcagtatcattggactataaacaagtgacagttattactgacgccacagctgctgctacacctgatatacacattgcaaacatacttgacatgaaaaatattggagtagcaacccctacattggaagaatggtgccaatctacatag

SEQ ID NO: 14 - protein, 194 aa, Nicotiana tabacum
MASELNVGKWKNVALLVIDMQKDFIFGPMQVKGGQAIVPNVIKAVEVARNCGIHIVWVVRENDPLGRDVELFRRHFKPKLASKGSVGAELVDGLVMDVEEDYKLVKTRFSAFLNTHLHSYLQTNGITDLVFTGVQTPNCIRQTVFDAVSLDYKQVTVITDATAAATPDIHIANILDMKNIGVATPTLEEWCQST

SEQ ID NO: 15 - DNA, 510 bp, Nicotiana tabacum
  source 1..510: mol_type = other DNA
atggccacggaatatgtaggcaaatggaagaacgtagctctccttgtcatagacatgcagatagacttcatatttggccctatgcaagtaaaaggaggtcaagctatcgtccctaacgttatcaaagctgttgaggttgcgagaaactgtggcattcacatcgtttgggttgtacgggagaatgatccattagggagagatgttgaattatttcgtcggcaccttaaaccaaagctggcatcaaagggtagtgtaggggctgaactaattgatgggctcgttatggacgtagaaaaggattacaagctggtcaaaacacgtttcagtgcatttcttaacacgcaccttcactcatattttcagaccaatggcattactaatctggtcttcaccggtgtccaaacaccaaattgcatacggcaaactgtgtttgatgcagtatcattggactataaacaagtgacagttattactgacgccacagctgctgctacacctgatatacacatt

SEQ ID NO: 16 - protein, 170 aa, Nicotiana tabacum
MATEYVGKWKNVALLVIDMQIDFIFGPMQVKGGQAIVPNVIKAVEVARNCGIHIVWVVRENDPLGRDVELFRRHLKPKLASKGSVGAELIDGLVMDVEKDYKLVKTRFSAFLNTHLHSYFQTNGITNLVFTGVQTPNCIRQTVFDAVSLDYKQVTVITDATAAATPDIHI

SEQ ID NO: 17 - DNA, 603 bp, Nicotiana tabacum
  source 1..603: mol_type = other DNA
atggcctcagaaaatggttgtaaatggaacaaaaccgctcttcttgtcatagacatgcagaaagattttgtactaccaggtgctcctatgctagttaaaggaggcgaagctattgttcctaacgttatcaagactgttgaggttgcaagaaaccgttgcatccccattatttgggttgtccgtgagcatgatccattaggtagagatgttgaattgtttcgtcggcatttatatggtgatgggaaaccaaaacctacatcaaagggtagtgtcggggcagaattggttgatgggcttgttattcaggaagatgattacaaattggtgaaaacccggttcagtgcattttttaacacgaaccttcattcgtatcttcagggcattggcattaccaacttggtcattattggtgttcaaactccaaattgcatacggcagactgtctttgatgctgtagcattggactatgaacgtgttacggtcattatagatgccacagctgctgctactcctgatatacatatcgcaaacataattgacatgaaaaatgtaggggtggtgactcctacgttagaagaatggtgccagtctatggagcaatag

SEQ ID NO: 18 - protein, 200 aa, Nicotiana tabacum
MASENGCKWNKTALLVIDMQKDFVLPGAPMLVKGGEAIVPNVIKTVEVARNRCIPIIWVVREHDPLGRDVELFRRHLYGDGKPKPTSKGSVGAELVDGLVIQEDDYKLVKTRFSAFFNTNLHSYLQGIGITNLVIIGVQTPNCIRQTVFDAVALDYERVTVIIDATAAATPDIHIANIIDMKNVGVVTPTLEEWCQSMEQ

SEQ ID NO: 19 - DNA, 612 bp, Nicotiana tabacum
  source 1..612: mol_type = other DNA
atggcctcagaaaatggttgtaaatggaacaaaactgctcttcttgtcatagacatgcagaaagattttgtactaccaggtgctcctatgctagttaaaggaggcgaagctattgttcctaacgttatcaagaccgttgaggttacaagaagccgcggcatccccattatttgggttgtccgtgagcatgatcaattaggtagagatgttgaattgtttcgtcggcatttatatggtgatggaaaaccaaaaccaacatcaaagggtagtgtgggggcagagctagttgatgggcttgttattgaggaagatgattacaaattggtgaaaacacggttcagtgcattttttaacacgaaccttcattcgtatcttcagggcattggcattaccaacttggtcattattggtgttcaaactccaaactgcatacggcagactgtctttgatgctgtagcattggactatgaacgtgttacggtcattatagatgccacagctgctgctactcctgatatacatatcgcgaacatatttgacatgaaaaatgtgggggtggcaactcctacgttagaagaatggtgccggtctaaggagcaatacaagtgctag

SEQ ID NO: 20 - protein, 203 aa, Nicotiana tabacum
MASENGCKWNKTALLVIDMQKDFVLPGAPMLVKGGEAIVPNVIKTVEVTRSRGIPIIWVVREHDQLGRDVELFRRHLYGDGKPKPTSKGSVGAELVDGLVIEEDDYKLVKTRFSAFFNTNLHSYLQGIGITNLVIIGVQTPNCIRQTVFDAVALDYERVTVIIDATAAATPDIHIANIFDMKNVGVATPTLEEWCRSKEQYKC

SEQ ID NO: 21 - DNA, 59 bp, synthetic construct
  misc_feature 1..59: note = RNAi plasmid construct
  source 1..59: mol_type = other DNA
acagatgctactgctacttcctcagcagaattgcatgatgctacactgaagaacttagc

SEQ ID NO: 22 - DNA, 26 bp, synthetic construct
  misc_feature 1..26: note = forward primer
  source 1..26: mol_type = other DNA
ttcctcatcatacaggaaatatgaaa

SEQ ID NO: 23 - DNA, 22 bp, synthetic construct
  misc_feature 1..22: note = reverse primer
  source 1..22: mol_type = other DNA
actcgtagagcatgccgtagtc

SEQ ID NO: 24 - DNA, 231 bp, Nicotiana tabacum
  source 1..231: mol_type = other DNA
atggcttcctcatcatacaggaaatatgaaacccgaaagcgaaatccagacccgaaatctgcggttcttttagtgatagacatacagaaccacttctactccatggccaagcctattctccccgctatcaacaccaccatcgacctctgccgacaggcttccgtgcctgtgatattcacgcgccaccgtcacaagtcaccggatgactacggcatgctctacgagtggtag

SEQ ID NO: 25 - protein, 76 aa, Nicotiana tabacum
MASSSYRKYETRKRNPDPKSAVLLVIDIQNHFYSMAKPILPAINTTIDLCRQASVPVIFTRHRHKSPDDYGMLYEW

SEQ ID NO: 26 - DNA, 231 bp, Nicotiana tabacum
  source 1..231: mol_type = other DNA
atggcttcctcatcatacaggaaatatgaaacccgaaagcgaaatccagacccgaaatctgcggttcttttagtgatagatatgcagaactacttctactccatggccaagcctattctccccgctatcaacactaccatcgacctctgccgacaggcttccgtgcctgtgatattcacgcgccaccgtcacaagtcaccggatgactacggcatgctctacgagtggtag

SEQ ID NO: 27 - protein, 76 aa, Nicotiana tabacum
MASSSYRKYETRKRNPDPKSAVLLVIDMQNYFYSMAKPILPAINTTIDLCRQASVPVIFTRHRHKSPDDYGMLYEW

(19) United States
(12) Patent Application Publication
(10) Pub. No.: US 2025/0084427 A1
(43) Pub. Date: Mar. 13, 2025

(54) MODULATION OF NICOTINE PRODUCTION BY ALTERATION OF NICOTINAMIDASE EXPRESSION OR FUNCTION IN PLANTS

(71) Applicant: PHILIP MORRIS PRODUCTS S.A., Neuchâtel (CH)

(72) Inventors: Lucien BOVET, La Chaux-de-Fonds (CH); Aurore HILFIKER, Salavaux (CH)

(21) Appl. No.: 18/722,452

(22) PCT Filed: Dec. 15, 2022
(86) PCT No.: PCT/EP2022/086183

§ 371 Date: Jun. 20, 2024

(30) Foreign Application Priority Data
Dec. 21, 2021 (EP) 21216648.2

(51) Int. Cl. C12N 15/82; A24B 3/12; A24B 15/10; C12N 9/80

(52) U.S. Cl. CPC C12N 15/8243; A24B 3/12; A24B 15/10; C12N 9/80; C12Y 305/01019

(57) ABSTRACT

There is described herein a plant cell having modulated expression or activity of a nicotinamidase enzyme, said plant cell comprising: (a) a polynucleotide sequence comprising, consisting or consisting essentially of a sequence encoding the nicotinamidase enzyme; or (b) a polypeptide encoded by the polynucleotide set forth in (a); wherein said plant cell comprises at least one modification which modulates the expression or activity of the polynucleotide or the polypeptide as compared to a control plant cell in which the expression or activity of the polynucleotide or polypeptide has not been modified. Furthermore, said poly nucleotide (a) of said plant cell comprises: (i) a polynucleotide comprising, consisting or consisting essentially of a sequence having at least 70% sequence identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 SEQ ID NO:24 or SEQ ID NO:26; and/or (ii) a construct, vector or expression vector comprising the isolated polynucleotide set forth in (i) or set forth in SEQ ID NO:21. Furthermore, wherein said polypeptide (b) of said plant cell comprises: a polypeptide encoded by the polynucleotide set forth above; and/or a polypeptide comprising, consisting or consisting essentially of a sequence having at least 90% sequence identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:25 or SEQ ID NO:27.

### FIELD OF THE INVENTION

The present invention discloses the polynucleotide sequences of genes encoding nicotinamidases from plants, and variants, homologues and fragments thereof. The polypeptide sequences encoded thereby and variants, homologues and fragments thereof, are also disclosed. The modulation of the expression of the one or more nicotinamidase genes or the function or activity of the nicotinamidase polypeptide(s) encoded thereby to modulate the level of tobacco in a plant or part thereof is also disclosed. In particular, the modified plant can be Nicotiana tabacum.

### BACKGROUND

There has been a longstanding view that cigarettes with reduced levels of nicotine could reduce nicotine dependence, thus reducing daily consumption, reducing the uptake of smoking and increasing attempts at quitting and increasing successful quitting (Benowitz and Henningfield, 1994. The New England Journal of Medicine, 331 (2): 123-125). This could reduce tobacco exposure and levels of smoking-associated morbidity or mortality.

Previous studies have attempted to modulate the levels of alkaloids in Nicotiana tabacum by targeting the genes involved in the alkaloid synthesis pathways. In particular, downregulation of ornithine decarboxylase in N. tabacum using RNAi has been found to result in reduced nicotine content and an increased anatabine content (DeBoer et al., 2011. Phytochemistry, 72:344-355, DeBoer et al., 2013 supra, Dalton et al., 2016. Journal of Experimental Botany, 67 (11): 3367-3381), A more recent study (Martinez et al. 2020. Planta 251 (4): 92) contemplates that multiple different biosynthetic pathways catalysed by a variety of different enzymes may feed substrate towards nicotine biosynthesis in commercial cultivars of tobacco. There is a continuing need in the art for commercially viable plants with modified levels of nicotine, especially Nicotiana tabacum. In particular, there is consumer demand for low nicotine tobacco and tobacco products due to perceived health benefits. The present invention seeks to address these and other needs.

### SUMMARY OF THE INVENTION

The present inventors have determined that modulating the expression of nicotinamidases in plants can result in modulated levels of nicotine therein. Advantageously, the level of nicotine modulation is observed without any significant impact on the growth phenotype of the host plant, which is suitably Nicotiana tabacum because this species is important in the context of the commercial production of tobacco.

Nicotinamidases convert nicotinamide to nicotinic acid. This reaction is an important step in the production of nicotinamide adenine dinucleotide (NAD). Hence nicotinamidases play an important role in NAD+ homeostasis in many prokaryotes, plants, and non-vertebrates (e.g., fungi, nematodes, and insects) by being part of the nicotinamide salvage pathway. In Arabidopsis thaliana, four genes, AtNIC1 to 4, are known to be active nicotinamidases. The gene At2g22570 encodes a nicotinamidase (AtNIC1), which is expressed in all tissues, with a highest level observed in roots and stems. Mutant plants have been shown to exhibit lower levels of NAD and NADP under normal growth conditions, indicating a function of AtNIC1 in a yeast-type NAD salvage pathway (Wang and Pichersky, 2007 Plant. J. 49 (6): 1020-9). Schippers et al. (2008. Plant Cell, 20 (10): 2909-2925), confirmed the function of AtNIC1 in the NAD salvage pathway active in the leaf.

Nicotinamidase is also part of the pyridine nucleotide cycle proposed by Wagner (Wagner et al., 1986. Planta, 167 (2): 226-32) to be involved in the nicotinic acid synthesis via the recycling of NAD. However, previously this enzyme has not been investigated in relation to tobacco synthesis because the knockout of a gene with such a crucial role in metabolism would have been expected to have a significant detrimental effect on plant metabolism.

In enzymology, a nicotinamidase (EC 3.5.1.19) is an enzyme that catalyses the chemical reaction between the two substrates nicotinamide and water, to produce nicotinate and ammonia. Nicotinamide is incorporated into NADH and NADPH and is a cofactor in a wide variety of enzymatic oxidation reduction reactions, including glycolysis, the citric acid cycle, and the electron transport chain. This makes the recycling of nicotinamide essential for the maintenance of cell activities. Most studies performed to date have however been focused on the pathways and enzymes of the NAD salvage pathway in yeast and animals, with few studies in plants.

The present inventors are the first to study the activity of nicotinamidases in their alternative pathway of nicotine synthesis in plants, though their role in nicotine synthesis via the pyridine nucleotide cycle was previously described by Wagner et al. (1986 see above). Disruption of nicotinamidase was previously considered likely to have a large negative effect on plant growth, and has therefore not been previously investigated. Overexpression of a nicotinamidase gene in Arabidopsis is known to increase plant biomass in addition to drought tolerance (Ahmad et al., 2021. Plant Mol Biol. 107:63-84), and therefore reduced expression would be expected to reduce biomass in plants.

The inventors have identified ten nicotinamidases in tobacco (described herein as SEQ ID NOs: 1, 3, 5, 7, 9, 11, 13, 15, 17 and 19) and the polypeptides they encode (described herein as SEQ ID Nos: 2, 4, 6, 8, 10, 12, 14, 16, 18 and 20).

When two nicotinamidases (NIC3-S and NIC3-T, as represented in SEQ ID NOs: 9 and 11) are silenced in Nicotiana tabacum, there is a significant reduction of nicotine levels in leaves, with only a slight but non-significant reduction in biomass. This demonstrates that nicotinamidase modulation can advantageously be used to alter the nicotine content of tobacco and products thereof.

In a first aspect, there is described a plant cell having modulated expression or activity of a nicotinamidase enzyme, the nicotinamidase enzyme comprising, consisting or consisting essentially of: (a) a polynucleotide sequence comprising, consisting or consisting essentially of a sequence encoding the nicotinamidase enzyme; or (b) a polypeptide encoded by the polynucleotide set forth in (a); wherein said plant cell comprises at least one modification which modulates the expression or activity of the polynucleotide or the polypeptide as compared to a control plant cell in which the expression or activity of the polynucleotide or polypeptide has not been modified.

Suitably, said plant cell is a non-naturally occurring or transgenic plant cell. Suitably, said plant cell is a tobacco plant cell, most suitably, a Nicotiana plant cell, most suitably, a Nicotiana tabacum plant cell.

Suitably, said polynucleotide (a) comprises: (i) a polynucleotide comprising, consisting or consisting essentially of a sequence having at least 70% sequence identity to SEQ ID NO:1, SEQ ID NO:3, SEQ ID NO:5, SEQ ID NO:7, SEQ ID NO:9, SEQ ID NO: 11, SEQ ID NO: 13, SEQ ID NO: 15, SEQ ID NO: 17, SEQ ID NO: 19 SEQ ID NO:24 or SEQ ID NO:26; and/or (ii) a construct, vector or expression vector comprising the isolated polynucleotide set forth in (i) or set forth in SEQ ID NO:21,

Suitably, said polypeptide (b) comprises: a polypeptide encoded by the polynucleotide set forth in (i) or (ii) above; and/or a polypeptide comprising, consisting or consisting essentially of a sequence having at least 90% sequence identity to SEQ ID NO:2, SEQ ID NO:4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:25 or SEQ ID NO:27.

Suitably said nicotinamidase enzyme is NIC3. Suitably said nicotinamidase enzyme is NIC3-S and NIC3-T, as represented in SEQ ID NOs: 9 and 11.

Suitably, the plant cell set forth above comprises a polynucleotide comprising, consisting or consisting essentially of a sequence having at least 80% sequence identity to SEQ ID NOs: 1, 3, 5, 7, 9, 11, 13, 15, 17, 19, 24 or 26 suitably, wherein the plant cell comprises a polynucleotide comprising, consisting or consisting essentially of a sequence having at least 85% sequence identity to SEQ ID NOs: 1, 3, 5, 7, 9, 11, 13, 15, 17, 19, 24 or 26; or wherein said plant cell comprises a polypeptide comprising, consisting or consisting essentially of a sequence having at least 95% sequence identity to SEQ ID NOs: 2, 4, 6, 8, 10, 12, 14, 16, 18, 20, 25 or 27 or at least 94% sequence identity to or SEQ ID NOs: 2, 4, 6, 8, 10, 12, 14, 16, 18, 20, 25 or 27 suitably, wherein the plant cell comprises a polypeptide comprising, consisting or consisting essentially of a sequence having at least 93% sequence identity to SEQ ID NOs: 2, 4, 6, 8, 10, 12, 14, 16, 18, 20, 25 or 27.

Suitably, the at least one modification of the plant cell is a modification of the plant cell's genome, or a modification of the construct, vector or expression vector, or a transgenic modification; suitably, wherein the modification of the plant cell's genome, or the modification of the construct, vector, or expression vector is a mutation or edit, most suitably wherein the modification of the plant cell's genome, or the modification of the construct, vector, or expression vector is a truncation. Suitably said truncation is caused by a mutation encoding a stop codon, most suitably wherein said mutation in a polynucleotide causes the translated polypeptide to be truncated (i.e. shorter) as compared to a control plant cell in which the stop codon has not been introduced.

Suitably, the modification of the plant cell decreases the expression or activity of the polynucleotide or the polypeptide as compared to the control plant cell; suitably, wherein the plant cell comprises an interference polynucleotide comprising a sequence that is at least 80% complementary to at least 50 nucleotides of an RNA transcribed from the polynucleotide of (i) as described above.

Suitably, the modulated expression or activity of the polynucleotide or the polypeptide of the plant cell modulates the level of nicotine in cured or dried leaf derived from the plant cell as compared to the level of nicotine in cured or dried leaf derived from a control plant, suitably wherein the level of nicotine in cured or dried leaf derived from the plant cell is decreased as compared to the level of nicotine in cured or dried leaf derived from a control plant. Additionally or alternatively, the modulated expression or activity of the polynucleotide or the polypeptide modulates the level of ammonia in cured or dried leaf derived from the plant cell as compared to the level of ammonia in cured or dried leaf derived from a control plant. Suitably, the level of ammonia in cured or dried leaf derived from the plant cell is decreased as compared to the level of ammonia in cured or dried leaf derived from a control plant

Suitably the modulated expression or activity of the polynucleotide or the polypeptide modulates the level of ammonia in cured or dried leaf derived from the plant cell as compared to the level of ammonia in cured or dried leaf derived from a control plant, suitably wherein the level of ammonia in cured or dried leaf derived from the plant cell is decreased as compared to the level of ammonia in cured or dried leaf derived from a control plant.

In a second aspect, there is described a plant or part thereof comprising the plant cell according to the first aspect; suitably, wherein the amount of nicotine is modified in at least a part of the plant as compared to a control plant or part thereof; suitably wherein the amount of nicotine is reduced in at least a part of the plant as compared to a control plant or part thereof, suitably wherein the amount of nicotine is reduced in at least the leaves of the plant as compared to the leaves of a control plant or part thereof.

In a third aspect, there is described plant material, cured plant material, or homogenized plant material, derived from the plant or part thereof of the second aspect, suitably wherein the cured plant material is air-cured or sun-cured or flue-cured plant material; suitably, wherein the plant material, cured plant material, or homogenized plant material comprises biomass, seed, stem, flowers, or leaves from the plant or part thereof of the second aspect.

In a fourth aspect, there is described a tobacco product comprising the plant cell of the first aspect, a part of the plant of the second aspect, or the plant material of the third aspect, suitably wherein said tobacco product comprises reduced nicotine content, suitably wherein said tobacco product comprises reduced nicotine content compared to a control tobacco product suitably wherein said tobacco product comprises reduced ammonia content, suitably wherein said tobacco product comprises reduced ammonia content compared to a control tobacco product.

In a fifth aspect, a method is provided for producing the plant of the second aspect, comprising the steps of: (a) providing a plant cell of any comprising a polynucleotide comprising, consisting or consisting essentially of a polynucleotide comprising, consisting or consisting essentially of a sequence having at least 80% sequence identity to SEQ ID NOs: 1, 3, 5, 7, 9, 11, 13, 15, 17, 19, 24 or 26; (b) modifying the plant cell to modulate the expression of said polynucleotide as compared to a control plant cell; and (c) propagating the plant cell into a plant, suitably wherein step (c) comprises cultivating the plant from a cutting or seedling comprising the plant cell. Optionally the step of modifying the plant cell comprises introducing a stop codon to the polynucleotide comprising, consisting or consisting essentially of a sequence having at least 80% sequence identity to SEQ ID NOs: 1, 3, 5, 7, 9, 11, 13, 15, 17, 19, 24 or 26, suitably wherein introduction of said stop codon causes truncation of polypeptide encoded by the polynucleotide comprising, consisting or consisting essentially of a sequence having at least 80% sequence identity to SEQ ID NOs: 1, 3, 5, 7, 9, 11, 13, 15, 17, 19, 24 or 26.

Suitably, the step of modifying the plant cell comprises modifying the genome of the cell by genome editing or genome engineering; suitably, wherein the genome editing or genome engineering is selected from CRISPR/Cas technology, zinc finger nuclease-mediated mutagenesis, chemical or radiation mutagenesis, homologous recombination, oligonucleotide-directed mutagenesis and meganuclease-mediated mutagenesis.

Suitably, the step of modifying the plant cell comprises transfecting the cell with a construct comprising a polynucleotide comprising, consisting, or consisting essentially of a sequence having at least 80% sequence identity to SEQ ID NO: 21 operably linked to a promoter; and/or wherein the step of modifying the plant cell comprises introducing an interference polynucleotide comprising a sequence that is at least 80% complementary to an RNA transcribed from the polynucleotide of (i) above into the cell; suitably, wherein the plant cell is transfected with a construct expressing an interference polynucleotide comprising a sequence that is at least 80% complementary to at least 50 nucleotides of an RNA transcribed from the polynucleotide of (i) above.

Suitably, the step of modifying the plant cell comprises introducing a stop codon to the polynucleotide comprising, consisting or consisting essentially of a sequence having at least 80% sequence identity to SEQ ID NOs: 1, 3, 5, 7, 9, 11, 13, 15, 17, 19, 24 or 26; suitably, wherein introduction of said stop codon causes the truncation of the polypeptide encoded by the polynucleotide comprising, consisting or consisting essentially of a sequence having at least 80% sequence identity to SEQ ID NOs: 1, 3, 5, 7, 9, 11, 13, 15, 17, 19, 24 or 26. Suitably the stop codon is a mutation from TGG (Tryptophan) to TAG, most suitably said stop codon is introduced to the 77th codon of the polynucleotide comprising, consisting or consisting essentially of a sequence having at least 99%, 95%, 90%, 85% or 80%, sequence identity to SEQ ID NOs: 1, 3, 5, 7, 9, 11, 13, 15, 17, 19. Suitably, the step of modifying the plant cell comprises introducing a stop codon to the polynucleotide comprising, consisting or consisting essentially of a sequence having at least 99%, 98%, 97%, 95%, 90%, 85% or 80% sequence identity to SEQ ID NO:24 or SEQ ID NO:26.

Suitably the introduction of said stop codon causes the polypeptide encoded by the polynucleotide comprising, consisting or consisting essentially of a sequence having at least 80% sequence identity to SEQ ID NOs: 1, 3, 5, 7, 9, 11, 13, 15, 17, 19 to be truncated by 1%, 2%, 5%, 10%, 15%, 20%, 25%, 30%, 35% 40%, 45%, 50%, 55%, 60%, 65% or 70% in length. Suitably the introduction of said stop codon causes the polypeptide encoded by the truncated polynucleotide to comprise, consist or consist essentially of a sequence having at least 95%, 90%, 85% or 85% sequence identity to SEQ ID NOs: 2, 4, 6, 8, 10, 12, 14, 16, 18, 20, 25 or 27. Suitably said polypeptide is truncated.

Suitably the introduction of a stop codon causes the polypeptide encoded by the polynucleotide comprising, consisting or consisting essentially of a sequence having at least 80% sequence identity to SEQ ID NOs: 1, 3, 5, 7, 9, 11, 13, 15, 17, 19 to be truncated and to be truncated by 1%, 2%, 5%, 10%, 15%, 20%, 25%, 30%, 35% 40%, 45%, 50%, 55%, 60%, 65% or 70% in length. Suitably said truncated polypeptide comprises, consists or consists essentially of a sequence having at least 95%, 90%, 85% or 85% sequence identity to SEQ ID NOs: 2, 4, 6, 8, 10, 12, 14, 16, 18 or 20. Suitably said truncated polypeptide comprises, consists or consists essentially of a sequence having at least 95%, 90%, 85% or 85% sequence identity to SEQ ID NOs: 2, 4, 6, 8, 10, 12, 14, 16, 18 or 20 wherein said polypeptide is truncated after amino acid 76 or the amino acid homologous to amino acid 76 of SEQ ID NO: 10 or SEQ ID NO: 12. Suitably said truncated polypeptide comprises, consists or consists essentially of a sequence having at least 95%, 90%, 85% or 85% sequence identity to SEQ ID NO: 25 or SEQ ID NO:27.

In a sixth aspect there is provided a tobacco product comprising a reduced nicotine level, suitably wherein said tobacco product comprises a reduced nicotine level compared to a control tobacco product, suitably wherein said tobacco product comprises a nicotine level reduced by at least 10%, 15%, 20%, 25%, 30%, 25%, 40%, 45%, 50%, 55%, 60%, 65%, 70% or 75% compared to a control tobacco product. Optionally said tobacco product further comprises reduced ammonia level, suitably wherein said tobacco product comprises reduced ammonia level compared to a control tobacco product. suitably wherein said tobacco product comprises an ammonia level reduced by at least 10%, 15%, 20%, 25%, 30%, 25%, 40%, 45%, 50%, 55%, 60%, 65%, 70% or 75% compared to a control tobacco product. Preferably said tobacco product comprises a plant cell according to a previous aspect.

In a seventh aspect, a method is provided for producing cured plant material with an altered amount of nicotine derived therefrom or an altered amount of nicotine as compared to control plant material, comprising the steps of: (a) providing a plant or part thereof according to the second aspect or the plant material according to this third aspect; (b) optionally harvesting the plant material therefrom; and (c) curing the plant material; suitably, wherein the plant material comprises cured leaves, cured stems or cured flowers, or a mixture thereof; and/or wherein the curing method is selected from the group consisting of air curing, fire curing, smoke curing, and flue curing.

### SOME ADVANTAGES

The present invention allows the production of tobacco from tobacco species such as Nicotiana tabacum and Nicotiana rustica wherein the nicotine content is modulated, most suitably wherein it is reduced.

Reduced nicotine tobacco is thought to reduce nicotine dependence in tobacco users, thus reducing daily consumption.

The present invention shows that knock down of nicotinamidase genes significantly affects nicotine biosynthesis, with plants showing up to a 90% reduction in nicotine content.

Mutants of NIC3 (SEQ ID NOs: 9 and 11, with the polypeptides shown as SEQ ID NOs: 10 and 12) in Nicotiana tabacum advantageously have a normal phenotype and the biomass is not or is only little affected. This can be important for the commercial production of tobacco when crop yields are important and when a normal leaf phenotype is required.

In addition, a reduction in ammonia content of the plants as a whole is also seen. In particular ammonia production in the leaves, where there is greatest nicotinamidase expression, is reduced most significantly if nicotinamidase expression is correspondingly reduced. Thus, the present invention provides tobacco plants and tobacco products comprising reduced nicotine and reduced ammonia.

Advantageously, the reduction of ammonia reduces irritation and inflammation in tobacco users, especially tobacco smokers.

To the best of the inventors' knowledge, nobody has shown such results before, and in view of its crucial role in energy metabolism in plants, nobody would have expected to be able to modify or knock out a nicotinamidase gene in any plant without extreme and undesirable effects on plant growth and viability.

### BRIEF DESCRIPTION OF THE DRAWINGS

FIG. 1 is a table showing expression of nicotinamidase and putative nicotinamidase genes in field grown Nicotiana tabacum. The data units are in fragment per kilobase of transcript per million mapped fragments (FPKM) resulting from RNAseq analyses with field ground tissues of the same plants.

FIG. 2 is a diagram of the gateway vector (pENTR221) used for plant transformation.

FIG. 3 is a graph showing NIC3-S(SEQ ID NO:9) and NIC3-T (SEQ ID NO:11) expression in Burley leaf under air-curing between 0 and 192 hours.

FIG. 4 is a graph showing gene expression of NIC3-S and NIC3-S (combined) in control (CT1-E427, n=15) and NIC3-RNAi transformed plants (T1-E427-15, n=4 and T1-E427-4, n=4) tobacco leaves after 48 hours air-curing. Analysis s performed by qPCR with the primers shown as SEQ ID NO:22 and SEQ ID NO:23 herein. An ANOVA HSD test is used for analysis, wherein p<0.001 is indicated by (**).

FIG. 5 is a graph showing the level of total alkaloids (TA) in control (CT1-E427, n=15) and NIC3-RNAi (T1-E427-15, n=4 and T1-E427-4, n=4) tobacco leaves after air-curing. An ANOVA HSD test is used for analysis, wherein p<0.001 is indicated by (***).

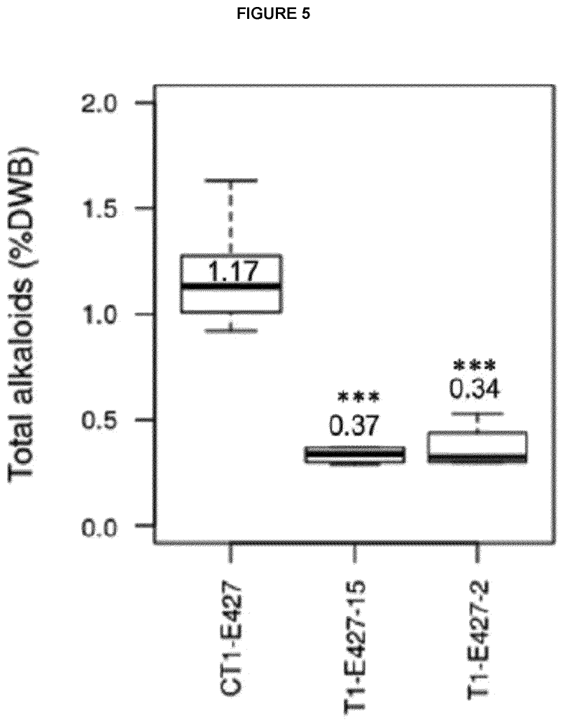

FIG. 6 is a two-panel graph. The left panel shows the level of ammonia in control (CT1-E427, n=15) and NIC3-RNAi (T1-E427-15, n=4 and T1-E427-4, n=4) tobacco leaves after air-curing. An ANOVA HSD test was used for analysis, wherein p<0.05 is indicated by (*). The right panel shows the fresh weight (FW) biomass of 4 mature leaves harvested at mid stalk position, (CT1-E427, n=15) and NIC3-RNAi (T1-E427-15, n=4 and T1-E427-4, n=4) tobacco leaves after air-curing. An ANOVA HSD test was used, wherein p<0.05 is indicated by (*).

FIG. 7 is a graph showing the nicotine content in control and NIC3-RNAi tobacco leaves after air-curing. The level of nicotine (mg/g) in control (CT1-E427-2, n=4, CT1-E427-4, n=4, CT1-E427-5, n=2) and NIC3-RNAi (T1-E427-15, n=4 and T1-E427-2, n=4) tobacco leaves after air-curing is indicated. The letters a-b indicates statistically significant differences based on one-way ANOVA analysis with Tukey's HSD test. Tukey's box plot was used with median as a center value.

FIG. 8 is a table showing the one-way ANOVA analysis with Tukey's HSD test of comparison of the nicotine content between control and NIC3-RNAi tobacco leaves after air curing.

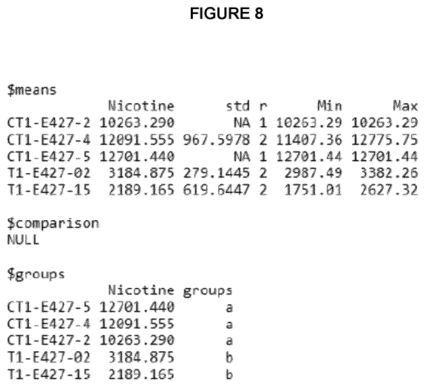

FIG. 9 is a table showing the level of nicotine in control and NIC3-RNAi tobacco leaves after air curing. The level of nicotine (% Dry Weight Basis) in control (CT1-E427, n=15) and NIC3-RNAi (T1-E427-15, n=4 and T1-E427-2, n=4) tobacco leaves after air-curing is indicated. SD (Standard Deviation) and an ANOVA HSD test were used for analysis, and the table indicates where p<0.05. FIG. 9 is a table of the data values presented in FIGS. 7 and 8, but expressed in % Dry Weight.

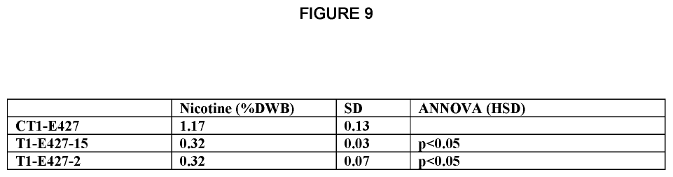

### DETAILED DESCRIPTION

Section headings as used in this disclosure are for organisation purposes and are not intended to be limiting.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The singular forms “a,” “and” and “the” include plural references unless the context clearly dictates otherwise.

The term “and/or” means (a) or (b) or both (a) and (b).

The present disclosure contemplates other embodiments “comprising,” “consisting of” and “consisting essentially of” the embodiments or elements presented herein, whether explicitly set forth or not.

For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used throughout the specification and the claims, the following terms have the following meanings:

“Coding sequence” or “polynucleotide encoding” means the nucleotides (RNA or DNA molecule) that comprise a polynucleotide which encodes a polypeptide. The coding sequence can further include initiation and termination signals operably linked to regulatory elements including a promoter and polyadenylation signal capable of directing expression in the cells of an individual or mammal to which the polynucleotide is administered. The coding sequence may be codon optimized.

“Complement” or “complementary” can mean Watson-Crick (for example, A-T/U and C-G) or Hoogsteen base pairing between nucleotides or nucleotide analogs. “Complementarity” refers to a property shared between two polynucleotides, such that when they are aligned antiparallel to each other, the nucleotide bases at each position will be complementary.

“Construct” refers to a double-stranded, recombinant polynucleotide fragment comprising one or more polynucleotides. The construct comprises a “template strand” base-paired with a complementary “sense or coding strand.” A given construct can be inserted into a vector in two possible orientations, either in the same (or sense) orientation or in the reverse (or anti-sense) orientation with respect to the orientation of a promoter positioned within a vector-such as an expression vector.

The term “control” in the context of a control plant or control plant cells means a plant or plant cells in which the expression, function or activity of one or more genes or polypeptides has not been modified (for example, increased or decreased) and so it can provide a comparison with a plant in which the expression, function or activity of the same one or more genes or polypeptides has been modified. As used herein, a “control plant” is a plant that is substantially equivalent to a test plant or modified plant in all parameters with the exception of the test parameters. For example, when referring to a plant into which a polynucleotide has been introduced, a control plant is an equivalent plant into which no such polynucleotide has been introduced. A control plant can be an equivalent plant into which a control polynucleotide has been introduced. In such instances, the control polynucleotide is one that is expected to result in little or no phenotypic effect on the plant. The control plant may comprise an empty vector. The control plant may correspond to a wild-type (WT) plant. The control plant may be a null segregant wherein the T1 segregant no longer possesses the transgene.

“Donor DNA” or “donor template” refers to a double-stranded DNA fragment or molecule that includes at least a portion of the gene of interest. The donor DNA may encode a fully-functional polypeptide or a partially-functional polypeptide.

“Endogenous gene or polypeptide” refers to a gene or polypeptide that originates from the genome of an organism and has not undergone a change, such as a loss, gain, or exchange of genetic material. An endogenous gene undergoes normal gene transmission and gene expression. An endogenous polypeptide undergoes normal expression.

“Enhancer sequences” refer to the sequences that can increase gene expression. These sequences can be located upstream, within introns or downstream of the transcribed region. The transcribed region is comprised of the exons and the intervening introns, from the promoter to the transcription termination region. The enhancement of gene expression can be through various mechanisms including increasing transcriptional efficiency, stabilization of mature mRNA and translational enhancement.

“Expression” refers to the production of a functional product. For example, expression of a polynucleotide fragment may refer to transcription of the polynucleotide fragment (for example, transcription resulting in mRNA or functional RNA) and/or translation of mRNA into a precursor or mature polypeptide. “Overexpression” refers to the production of a gene product in transgenic organisms that exceeds levels of production in a null segregating (or non-transgenic) organism from the same experiment.

“Functional” and “full-functional” describes a polypeptide that has biological function or activity. A “functional gene” refers to a gene transcribed to mRNA, which is translated to a functional or active polypeptide.

“Genetic construct” refers to DNA or RNA molecules that comprise a polynucleotide that encodes a polypeptide. The coding sequence can include initiation and termination signals operably linked to regulatory elements including a promoter and polyadenylation signal capable of directing expression.

“Genome editing” refers to changing an endogenous gene that encodes an endogenous polypeptide, such that polypeptide expression of a truncated endogenous polypeptide or an endogenous polypeptide having an amino acid substitution is obtained. Genome editing can include replacing the region of the endogenous gene to be targeted or replacing the entire endogenous gene with a copy of the gene that has a truncation or an amino acid substitution with a repair mechanism—such as HDR. Genome editing may also include generating an amino acid substitution in the endogenous gene by generating a double stranded break in the endogenous gene that is then repaired using NHEJ. NHEJ may add or delete at least one base pair during repair which may generate an amino acid substitution. Genome editing may also include deleting a gene segment by the simultaneous action of two nucleases on the same DNA strand in order to create a truncation between the two nuclease target sites and repairing the DNA break by NHEJ.

“Heterologous” with respect to a sequence means a sequence that originates from a foreign species, or, if from the same species, is substantially modified from its native form in composition and/or genomic locus by deliberate human intervention.

“Homology-directed repair” or “HDR” refers to a mechanism in cells to repair double strand DNA lesions when a homologous piece of DNA is present in the nucleus, mostly in G2 and S phase of the cell cycle. HDR uses a donor DNA or donor template to guide repair and may be used to create specific sequence changes to the genome, including the targeted addition of whole genes. If a donor template is provided along with the site specific nuclease, then the cellular machinery will repair the break by homologous recombination, which is enhanced several orders of magnitude in the presence of DNA cleavage. When the homologous DNA piece is absent, NHEJ may take place instead.

The terms “homology” or “similarity” refer to the degree of sequence similarity between two polypeptides or between two polynucleotide molecules compared by sequence alignment. The degree of homology between two discrete polynucleotides being compared is a function of the number of identical, or matching, nucleotides at comparable positions.

“Identical” or “identity” in the context of two or more polynucleotides or polypeptides means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. Percentage identity can be determined over the full length of a sequence. When comparing DNA and RNA, thymine (T) and uracil (U) may be considered equivalent. Identity may be determined manually or by using a computer sequence algorithm such as ClustalW, ClustalX, BLAST, FASTA or Smith-Waterman. The popular multiple alignment program ClustalW (Nucleic Acids Research (1994) 22, 4673-4680; Nucleic Acids Research (1997), 24, 4876-4882) is a suitable way for generating multiple alignments of polypeptides or polynucleotides. Suitable parameters for ClustalW maybe as follows: For polynucleotide alignments: Gap Open Penalty=15.0, Gap Extension Penalty=6.66, and Matrix=Identity. For polypeptide alignments: Gap Open Penalty=10. o, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment. Suitably, calculation of percentage identities is then calculated from such an alignment as (N/T), where N is the number of positions at which the sequences share an identical residue, and T is the total number of positions compared including gaps but excluding overhangs.

The term “increase” or “increased” refers to an increase of from about 10% to about 99%, or an increase of at least 10%, at least 20%, at least 25%, at least 30%, at least 40%, at least 50%, at least 60%, at least 70%, at least 75%, at least 80%, at least 90%, at least 95%, at least 98%, at least 99%, at least 100%, at least 150%, or at least 200% or more or more of a quantity or a function or an activity, such as but not limited to polypeptide function or activity, transcriptional function or activity, and/or polypeptide expression. The term “increased,” or the phrase “an increased amount” can refer to a quantity or a function or an activity in a modified plant or a product generated from the modified plant that is more than what would be found in a plant or a product from the same variety of plant processed in the same manner, which has not been modified. Thus, in some contexts, a wild-type plant of the same variety that has been processed in the same manner is used as a control by which to measure whether an increase in quantity is obtained.

The term “reduce” or “reduced” as used herein, refers to a reduction of from about 10% to about 99%, or a reduction of at least 10%, at least 20%, at least 25%, at least 30%, at least 40%, at least 50%, at least 60%, at least 70%, at least 75%, at least 80%, at least 90%, at least 95%, at least 98%, at least 99%, or at least 100% or, or at least 150%, or at least 200% more of a quantity or a function—such as polypeptide function, transcriptional function, or polypeptide expression. The term “reduced,” or the phrase “a reduced amount” can refer to a quantity or a function in a modified plant or a product generated from the modified plant that is less than what would be found in a plant or a product from the same variety of plant processed in the same manner, which has not been modified. Thus, in some contexts, a wild-type plant of the same variety that has been processed in the same manner is used as a control by which to measure whether a reduction in quantity is obtained.

The term “inhibit” or “inhibited” refers to a reduction of from about 98% to about 100%, or a reduction of at least 98%, at least 99%, but particularly of 100%, of a quantity or a function or an activity, such as but not limited to polypeptide function or activity, transcriptional function or activity, and/or polypeptide expression.

The term “introduced” means providing a polynucleotide (for example, a construct) or polypeptide into a cell. Introduced includes reference to the incorporation of a polynucleotide into a eukaryotic cell where the polynucleotide may be incorporated into the genome of the cell, and includes reference to the transient provision of a polynucleotide or polypeptide to the cell. Introduced includes reference to stable or transient transformation methods, as well as sexually crossing. Thus, “introduced” in the context of inserting a polynucleotide (for example, a recombinant construct/expression construct) into a cell, means “transfection” or “transformation” or “transduction” and includes reference to the incorporation of a polynucleotide into a eukaryotic cell where the polynucleotide may be incorporated into the genome of the cell (for example, chromosome, plasmid, plastid or mitochondrial DNA), converted into an autonomous replicon, or transiently expressed (for example, transfected mRNA).

The terms “isolated” or “purified” refer to material that is substantially or essentially free from components that normally accompany it as found in its native state. Purity and homogeneity are typically determined using analytical chemistry techniques such as polyacrylamide gel electrophoresis or high performance liquid chromatography. A polypeptide that is the predominant species present in a preparation is substantially purified. In particular, an isolated polynucleotide is separated from open reading frames that flank the desired gene and encode polypeptides other than the desired polypeptide. The term “purified” as used herein denotes that a polynucleotide or polypeptide gives rise to essentially one band in an electrophoretic gel. Particularly, it means that the polynucleotide or polypeptide is at least 85% pure, more suitably at least 95% pure, and most suitably at least 99% pure. Isolated polynucleotides may be purified from a host cell in which they naturally occur. Conventional polynucleotide purification methods known to skilled artisans may be used to obtain isolated polynucleotides. The term also embraces recombinant polynucleotides and chemically synthesized polynucleotides.

The term “level” refers to an amount, and is used interchangeably with “content”. Nicotine content or nicotine level is the amount of nicotine contained in the feature described. Said amount may suitably be measured by weight. Ammonia content or ammonia level is the amount of ammonia contained in the feature described. Said amount may suitably be measured by weight. Suitably the ammonia content is measured using a quantitative method, suitably the SKALAR method. The SKALAR method is an agreed ISO method by CORESTA (Cooperation Centre for Scientific Research Relative to Tobacco) for the quantification of ammonia in tobacco. It is method No. 79-Determination of Ammonia in Tobacco and Tobacco Products by lon Chromatographic Analysis (ISO 21045).

“Modulate” or “modulating” refers to causing or facilitating a qualitative or quantitative change, alteration, or modification in a process, pathway, function or activity of interest. Without limitation, such a change, alteration, or modification may be an increase or decrease in the relative process, pathway, function or activity of interest. For example, gene expression or polypeptide expression or polypeptide function or activity can be modulated. Typically, the relative change, alteration, or modification will be determined by comparison to a control. “Nicotinamidase” refers to an enzyme or polypeptide, or a polynucleotide or gene encoding said enzyme or polypeptide, wherein said enzyme or polypeptide catalyses the chemical reaction:

Nicotinamide+H₂O [symbol] nicotinate+NH₃

Nicotinamidases described herein belong to the enzyme class nicotinamide amidohydrolase, also known as nicotinamide deaminase or nicotinamide amidase, with EC number 3.5.1.19. “Nicotinamide”, also known as niacinamide or NAM, is a water soluble form of the essential human nutrient B₃ having the structure shown below.

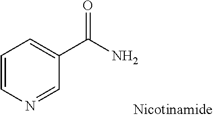

“Nicotinate”, also known as nicotinic acid or niacin, is the essential human nutrient B₃ and has the structure shown below.

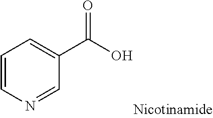

“Ammonia”, as produced by the reaction catalysed by Nicotinamidases as described above, has the chemical formula NH₃

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The term ‘non-naturally occurring’ describes an entity—such as a polynucleotide, a genetic mutation, a polypeptide, a plant, a plant cell and plant material—that is not formed by nature or that does not exist in nature. Such non-naturally occurring entities or artificial entities may be made, synthesized, initiated, modified, intervened, or manipulated by methods described herein or that are known in the art. Such non-naturally occurring entities or artificial entities may be made, synthesized, initiated, modified, intervened, or manipulated by man. Thus, by way of example, a non-naturally occurring plant, a non-naturally occurring plant cell or non-naturally occurring plant material may be made using traditional plant breeding techniques—such as backcrossing—or by genetic manipulation technologies—such as antisense RNA, interfering RNA, meganuclease and the like. By way of further example, a non-naturally occurring plant, a non-naturally occurring plant cell or non-naturally occurring plant material may be made by introgression of or by transferring one or more genetic mutations (for example one or more polymorphisms) from a first plant or plant cell into a second plant or plant cell (which may itself be naturally occurring), such that the resulting plant, plant cell or plant material or the progeny thereof comprises a genetic constitution (for example, a genome, a chromosome or a segment thereof) that is not formed by nature or that does not exist in nature. The resulting plant, plant cell or plant material is thus artificial or non-naturally occurring. Accordingly, an artificial or non-naturally occurring plant or plant cell may be made by modifying a genetic sequence in a first naturally occurring plant or plant cell, even if the resulting genetic sequence occurs naturally in a second plant or plant cell that comprises a different genetic background from the first plant or plant cell. In certain embodiments, a mutation is not a naturally occurring mutation that exists naturally in a polynucleotide or a polypeptide—such as a gene or a polypeptide. Differences in genetic background can be detected by phenotypic differences or by molecular biology techniques known in the art—such as polynucleotide sequencing, presence or absence of genetic markers (for example, microsatellite RNA markers).

“Oligonucleotide” or “polynucleotide” means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a given sequence under stringent hybridization conditions. Thus, a polynucleotide also encompasses a probe that hybridizes under stringent hybridization conditions. Polynucleotides may be single stranded or double stranded, or may contain portions of both double stranded and single stranded sequence. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Polynucleotides may be obtained by chemical synthesis methods or by recombinant methods. The specificity of single-stranded DNA to hybridize complementary fragments is determined by the “stringency” of the reaction conditions (Sambrook et al., Molecular Cloning and Laboratory Manual, Second Ed., Cold Spring Harbor (1989)). Hybridization stringency increases as the propensity to form DNA duplexes decreases. In polynucleotide hybridization reactions, the stringency can be chosen to favor specific hybridizations (high stringency), which can be used to identify, for example, full-length clones from a library. Less-specific hybridizations (low stringency) can be used to identify related, but not exact (homologous, but not identical), DNA molecules or segments. DNA duplexes are stabilised by: (1) the number of complementary base pairs; (2) the type of base pairs; (3) salt concentration (ionic strength) of the reaction mixture; (4) the temperature of the reaction; and (5) the presence of certain organic solvents, such as formamide, which decrease DNA duplex stability. In general, the longer the probe, the higher the temperature required for proper annealing. A common approach is to vary the temperature; higher relative temperatures result in more stringent reaction conditions. To hybridize under “stringent conditions” describes hybridization protocols in which polynucleotides at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the given sequence hybridize to the given sequence at equilibrium. Since the given sequences are generally present at excess, at Tm, 50% of the probes are occupied at equilibrium.

“Stringent hybridization conditions” are conditions that enable a probe, primer, or oligonucleotide to hybridize only to its specific sequence. Stringent conditions are sequence-dependent and will differ. Stringent conditions typically comprise: (1) low ionic strength and high temperature washes, for example 15 mM sodium chloride, 1.5 mM sodium citrate, 0.1% sodium dodecyl sulfate, at 50° C.; (2) a denaturing agent during hybridization, for example, 50% (v/v) formamide, 0.1% bovine serum albumin, 0.1% Ficoll, 0.1% polyvinylpyrrolidone, 50 mM sodium phosphate buffer (750 mM sodium chloride, 75 mM sodium citrate; pH 6.5), at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with a wash at 42° C. in 0.2×SSC (sodium chloride/sodium citrate) and 50% formamide at 55° C., followed by a high-stringency wash consisting of 0.1×SSC containing EDTA at 55° C.

Suitably, the conditions are such that sequences at least about 65%, 70%, 75%, 85%, 90%, 95%, 98%, or 99% homologous to each other typically remain hybridized to each other. “Moderately stringent conditions” use washing solutions and hybridization conditions that are less stringent, such that a polynucleotide will hybridize to the entire, fragments, derivatives, or analogs of the polynucleotide. One example comprises hybridization in 6×SSC, 5×Denhardt's solution, 0.5% SDS and 100 μg/mL denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (see Ausubel et al., Current Protocols in Molecular Biology, Volumes 1-3, John Wiley & Sons, Inc., Hoboken, N.J. (1993); Kriegler, Gene Transfer and Expression: A Laboratory Manual, Stockton Press, New York, N.Y. (1990); Perbal, A Practical Guide to Molecular Cloning, 2nd edition, John Wiley & Sons, New York, N.Y. (1988)). “Low stringent conditions” use washing solutions and hybridization conditions that are less stringent than those for moderate stringency, such that a polynucleotide will hybridize to the entire, fragments, derivatives, or analogs of the polynucleotide. A non-limiting example of low stringency hybridization conditions includes hybridization in 35% formamide, 5×SSC, 50 mM Tris HCl (pH 7.5), 5 mM EDTA, 0.02% PVP, 0.02% Ficoll, 0.2% BSA, 100 μg/mL denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross-species hybridizations, are well-described (see Ausubel et al., 1993; Kriegler, 1990).

“Operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. “Operably linked” refers to the association of polynucleotide fragments in a single fragment so that the function of one is regulated by the other. For example, a promoter is operably linked with a polynucleotide fragment when it is capable of regulating the transcription of that polynucleotide fragment.

The term “plant” refers to any plant at any stage of its life cycle or development, and its progenies. In one embodiment, the plant is a tobacco plant, which refers to a plant belonging to the genus Nicotiana. The term includes reference to whole plants, plant organs, plant tissues, plant propagules, plant seeds, plant cells and progeny of same. Plant cells include, without limitation, cells from seeds, suspension cultures, embryos, meristematic regions, callus tissue, leaves, roots, shoots, gametophytes, sporophytes, pollen, and microspores. Suitable species, cultivars, hybrids and varieties of tobacco plant are described herein.

“Polynucleotide”, “polynucleotide sequence” or “polynucleotide fragment” are used interchangeably herein and refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. A polynucleotide can be, without limitation, a genomic DNA, complementary DNA (cDNA), mRNA, or antisense RNA or a fragment(s) thereof. Moreover, a polynucleotide can be single-stranded or double-stranded, a mixture of single-stranded and double-stranded regions, a hybrid molecule comprising DNA and RNA, or a hybrid molecule with a mixture of single-stranded and double-stranded regions or a fragment(s) thereof. In addition, the polynucleotide can be composed of triple-stranded regions comprising DNA, RNA, or both or a fragment(s) thereof. A polynucleotide can contain one or more modified bases, such as phosphothioates, and can be a peptide nucleic acid (PNA). Generally, polynucleotides can be assembled from isolated or cloned fragments of cDNA, genomic DNA, oligonucleotides, or individual nucleotides, or a combination of the foregoing. Although the polynucleotides described herein are shown as DNA sequences, the polynucleotides include their corresponding RNA sequences, and their complementary (for example, completely complementary) DNA or RNA sequences, including the reverse complements thereof. The polynucleotides of the present disclosure are set forth in the accompanying sequence listing.

“Polypeptide” or “polypeptide sequence” refer to a polymer of amino acids in which one or more amino acid residues is an artificial chemical analogue of a corresponding naturally occurring amino acid, as well as to naturally occurring polymers of amino acids. The terms are also inclusive of modifications including, but not limited to, glycosylation, lipid attachment, sulfation, gamma-carboxylation of glutamic acid residues, hydroxylation and ADP-ribosylation. The polypeptides of the present disclosure are set forth in the accompanying sequence listing. “Promoter” means a synthetic or naturally-derived molecule which is capable of conferring, activating or enhancing expression of a polynucleotide in a cell. The term refers to a polynucleotide element/sequence, typically positioned upstream and operably-linked to a double-stranded polynucleotide fragment. Promoters can be derived entirely from regions proximate to a native gene of interest, or can be composed of different elements derived from different native promoters or synthetic polynucleotide segments. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents.

“Tissue-specific promoter” and “tissue-preferred promoter” as used interchangeably herein refer to a promoter that is expressed predominantly but not necessarily exclusively in one tissue or organ, but that may also be expressed in one specific cell. A “developmentally regulated promoter” refers to a promoter whose function is determined by developmental events. A “constitutive promoter” refers to a promoter that causes a gene to be expressed in most cell types at most times. An “inducible promoter” selectively expresses an operably linked DNA sequence in response to the presence of an endogenous or exogenous stimulus, for example by chemical compounds (chemical inducers) or in response to environmental, hormonal, chemical, and/or developmental signals. Examples of inducible or regulated promoters include promoters regulated by light, heat, stress, flooding or drought, pathogens, phytohormones, wounding, or chemicals such as ethanol, jasmonate, salicylic acid, or safeners.

“Recombinant” as used herein refers to an artificial combination of two otherwise separated segments of sequence—such as by chemical synthesis or by the manipulation of isolated segments of polynucleotides by genetic engineering techniques. The term also includes reference to a cell or vector, that has been modified by the introduction of a heterologous polynucleotide or a cell derived from a cell so modified, but does not encompass the alteration of the cell or vector by naturally occurring events (for example, spontaneous mutation, natural transformation or transduction or transposition) such as those occurring without deliberate human intervention.

“Recombinant construct” refers to a combination of polynucleotides that are not normally found together in nature. Accordingly, a recombinant construct may comprise regulatory sequences and coding sequences that are derived from different sources, or regulatory sequences and coding sequences derived from the same source, but arranged in a manner different than that normally found in nature. The recombinant construct can be a recombinant DNA construct. “Regulatory sequences” and “regulatory elements” as used interchangeably herein refer to polynucleotide sequences located upstream (5′ non-coding sequences), within, or downstream (3′ non-coding sequences) of a coding sequence, and which influence the transcription, RNA processing or stability, or translation of the associated coding sequence. Regulatory sequences include promoters, translation leader sequences, introns, and polyadenylation recognition sequences. The terms “regulatory sequence” and “regulatory element” are used interchangeably herein.

“Site-specific nuclease” refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), CRISPR/Cas9-based systems, and meganucleases.

The term “tobacco” is used in a collective sense to refer to tobacco crops (for example, a plurality of tobacco plants grown in the field and not hydroponically grown tobacco), tobacco plants and parts thereof, including but not limited to, roots, stems, leaves, flowers, and seeds prepared and/or obtained, as described herein. It is understood that “tobacco” includes Nicotiana tabacum plants and products thereof.

The term “tobacco products” refers to consumer tobacco products, including but not limited to, smoking materials (for example, cigarettes, cigars, and pipe tobacco), snuff, chewing tobacco, gum, and lozenges, as well as components, materials and ingredients for manufacture of consumer tobacco products. Suitably, these tobacco products are manufactured from tobacco leaves and stems harvested from tobacco and cut, dried, cured, and/or fermented according to conventional techniques in tobacco preparation. The tobacco in the tobacco products may be combined with a binder, as described herein.

“Transcription terminator”, “termination sequences”, or “terminator” refers to DNA sequences located downstream of a coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Transgenic” refers to any cell, cell line, callus, tissue, plant part or plant, the genome of which has been altered by the presence of a heterologous polynucleotide, such as a recombinant construct, including those initial transgenic events as well as those created by sexual crosses or asexual propagation from the initial transgenic event. The term does not encompass the alteration of the genome (chromosomal or extra-chromosomal) by conventional plant breeding methods or by naturally occurring events—such as random cross-fertilization, non-recombinant viral infection, non-recombinant bacterial transformation, non-recombinant transposition, or spontaneous mutation.

“Transgenic plant” refers to a plant which comprises within its genome one or more heterologous polynucleotides, that is, a plant that contains recombinant genetic material not normally found therein and which has been introduced into the plant in question (or into progenitors of the plant) by human manipulation. For example, the heterologous polynucleotide can be stably integrated within the genome such that the polynucleotide is passed on to successive generations. The heterologous polynucleotide can be integrated into the genome alone or as part of a recombinant construct. The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems and the like. Each heterologous polynucleotide may confer a different trait to the transgenic plant.

“Transcription activator-like effector” or “TALE” refers to a polypeptide structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules may be arranged in any order to assemble an array that recognizes a defined sequence. A binding specificity of a TALE DNA-binding domain is determined by the RVD array followed by a single truncated repeat of 20 amino acids. A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors, methyltransferases, integrases, nucleases, and recombinases.

“Transcription activator-like effector nucleases” or “TALENs” as used interchangeably herein refers to engineered fusion polypeptides of the catalytic domain of a nuclease, such as endonuclease FokI, and a designed TALE DNA-binding domain that may be targeted to a custom DNA sequence.

A “TALEN monomer” refers to an engineered fusion polypeptide with a catalytic nuclease domain and a designed TALE DNA-binding domain. Two TALEN monomers may be designed to target and cleave a TALEN target region.

“Transgene” refers to a gene or genetic material containing a gene sequence that has been isolated from one organism and is introduced into a different organism. This non-native segment of DNA may retain the ability to produce RNA or polypeptide in the transgenic organism, or it may alter the normal function of the transgenic organism's genetic code. The introduction of a transgene has the potential to change the phenotype of an organism. “Variant” with respect to a polynucleotide means: (i) a portion or fragment of a polynucleotide; (ii) the complement of a polynucleotide or portion thereof; (iii) a polynucleotide that is substantially identical to a polynucleotide of interest or the complement thereof; or (iv) a polynucleotide that hybridizes under stringent conditions to the polynucleotide of interest, complement thereof, or a polynucleotide substantially identical thereto.

“Variant” with respect to a peptide or polypeptide means a peptide or polypeptide that differs in sequence by the insertion, deletion, or conservative substitution of amino acids, but retain at least one biological function or activity. Variant may also mean a polypeptide that retains at least one biological function or activity. A conservative substitution of an amino acid, that is, replacing an amino acid with a different amino acid of similar properties (for example, hydrophilicity, degree and distribution of charged regions) is recognized in the art as typically involving a minor change.

The term “variety” refers to a population of plants that share constant characteristics which separate them from other plants of the same species. While possessing one or more distinctive traits, a variety is further characterized by a very small overall variation between individuals within that variety. A variety is often sold commercially.

“Vector” refers to a polynucleotide vehicle that comprises a combination of polynucleotide components for enabling the transport of polynucleotides, polynucleotide constructs and polynucleotide conjugates and the like. A vector may be a viral vector, bacteriophage, bacterial artificial chromosome or yeast artificial chromosome. A vector may be a DNA or RNA vector. Suitable vectors include episomes capable of extra-chromosomal replication such as circular, double-stranded nucleotide plasmids; linearized double-stranded nucleotide plasmids; and other vectors of any origin. An “expression vector” as used herein is a polynucleotide vehicle that comprises a combination of polynucleotide components for enabling the expression of polynucleotide(s), polynucleotide constructs and polynucleotide conjugates and the like. Suitable expression vectors include episomes capable of extra-chromosomal replication such as circular, double-stranded nucleotide plasmids; linearized double-stranded nucleotide plasmids; and other functionally equivalent expression vectors of any origin. An expression vector comprises at least a promoter positioned upstream and operably-linked to a polynucleotide, polynucleotide constructs or polynucleotide conjugate, as defined below.

“Zinc finger” refers to a polypeptide structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

“Zinc finger nuclease” or “ZFN” refers to a chimeric polypeptide molecule comprising at least one zinc finger DNA binding domain effectively linked to at least one nuclease or part of a nuclease capable of cleaving DNA when fully assembled.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and polypeptide and polynucleotide chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

In one embodiment, there is provided an isolated polynucleotide comprising, consisting or consisting essentially of a sequence having at least 60% sequence identity to any of the sequences described herein, including any of polynucleotides shown in the sequence listing.

Suitably, the isolated polynucleotide comprises, consists or consists essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity thereto.

Suitably, the polynucleotide(s) described herein encode an active polypeptide that has at least about 50%, 60%, 70%, 80%, 90% 95%, 96%, 97%, 98%, 99%, 100% or more of the function or activity of the polypeptide(s) shown in the sequence listing.

In another embodiment, there is provided an isolated polynucleotide comprising, consisting or consisting essentially of a polynucleotide having at least 60% sequence identity to SEQ ID NO. 1, SEQ ID NO:3, SEQ ID NO:5, SEQ ID NO:7, SEQ ID NO:9, SEQ ID NO: 11, SEQ ID NO: 13, SEQ ID NO: 15, SEQ ID NO: 17, SEQ ID NO: 19 SEQ ID NO:24 or SEQ ID NO:26. Suitably, the isolated polynucleotide comprises, consists or consist essentially of a sequence having at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO. 1, 3, 5, 7, 9, 11, 13, 15, 17, 19, 24 or 26.

In another embodiment, there is provided polynucleotides comprising, consisting or consisting essentially of polynucleotides with substantial homology (that is, sequence similarity) or substantial identity to SEQ ID NO. 1, 3, 5, 7, 9, 11, 13, 15, 17, 19, 24 or 26.

In another embodiment, there is provided fragments of SEQ ID NO. 1, 3, 5, 7, 9, 11, 13, 15, 17, 19, 24 or 26 with substantial homology (that is, sequence similarity) or substantial identity thereto that have at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO. 1, 3, 5, 7, 9, 11, 13, 15, 17, 19, 24 or 26.

In another embodiment, there is provided polynucleotides comprising a sufficient or substantial degree of identity or similarity to SEQ ID NO. 1, 3, 5, 7, 9, 11, 13, 15, 17, 19, 24 or 26 that encode a polypeptide that functions as a nicotinamidase.

In another embodiment, there is provided a polymer of polynucleotides which comprises, consists or consists essentially of a polynucleotide designated herein as SEQ ID NO. 1, 3, 5, 7, 9, 11, 13, 15, 17, 19, 24 or 26.

Suitably, the polynucleotides described herein encode members of the nicotinamidase family of enzyme.

A polynucleotide as described herein can include a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA or a fragment(s) thereof. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions or a fragment(s) thereof. In addition, the polynucleotide can be composed of triple-stranded regions comprising DNA, RNA, or both or a fragment(s) thereof. A polynucleotide can contain one or more modified bases, such as phosphothioates, and can be a peptide nucleic acid. Generally, polynucleotides can be assembled from isolated or cloned fragments of cDNA, genomic DNA, oligonucleotides, or individual nucleotides, or a combination of the foregoing. Although the polynucleotides described herein are shown as DNA sequences, they include their corresponding RNA sequences, and their complementary (for example, completely complementary) DNA or RNA sequences, including the reverse complements thereof.

A polynucleotide as described herein will generally contain phosphodiester bonds, although in some cases, polynucleotide analogues are included that may have alternate backbones, comprising, for example, phosphoramidate, phosphorothioate, phosphorodithioate, or O-methylphophoroamidite linkages; and peptide polynucleotide backbones and linkages. Other analogue polynucleotides include those with positive backbones; non-ionic backbones, and non-ribose backbones. Modifications of the ribose-phosphate backbone may be done for a variety of reasons, for example, to increase the stability and half-life of such molecules in physiological environments or as probes on a biochip. Mixtures of naturally occurring polynucleotides and analogues can be made; alternatively, mixtures of different polynucleotide analogues, and mixtures of naturally occurring polynucleotides and analogues may be made. A variety of polynucleotide analogues are known, including, for example, phosphoramidate, phosphorothioate, phosphorodithioate, O-methylphophoroamidite linkages and peptide polynucleotide backbones and linkages. Other analogue polynucleotides include those with positive backbones, non-ionic backbones and non-ribose backbones. Polynucleotides containing one or more carbocyclic sugars are also included.

Other analogues include peptide polynucleotides which are peptide polynucleotide analogues. These backbones are substantially non-ionic under neutral conditions, in contrast to the highly charged phosphodiester backbone of naturally occurring polynucleotides. This may result in advantages. First, the peptide polynucleotide backbone may exhibit improved hybridization kinetics. Peptide polynucleotides have larger changes in the melting temperature for mismatched versus perfectly matched base pairs. DNA and RNA typically exhibit a 2-4° C. drop in melting temperature for an internal mismatch. With the non-ionic peptide polynucleotide backbone, the drop is closer to 7-9° C. Similarly, due to their non-ionic nature, hybridization of the bases attached to these backbones is relatively insensitive to salt concentration. In addition, peptide polynucleotides may not be degraded or degraded to a lesser extent by cellular enzymes, and thus may be more stable.

Among the uses of the disclosed polynucleotides, and fragments thereof, is the use of fragments as probes in hybridisation assays or primers for use in amplification assays. Such fragments generally comprise at least about 10, 11, 12, 13, 14, 15, 16, 17, 18, 19 or 20 or more contiguous nucleotides of a DNA sequence. In other embodiments, a DNA fragment comprises at least about 10, 15, 20, 30, 40, 50 or 60 or more contiguous nucleotides of a DNA sequence. Thus, in one aspect, there is also provided a method for detecting a polynucleotide comprising the use of the probes or primers or both. Exemplary primers are described herein. The basic parameters affecting the choice of hybridization conditions and guidance for devising suitable conditions are described by Sambrook, J., E. F. Fritsch, and T. Maniatis (1989, Molecular Cloning: A Laboratory Manual, Cold Spring Harbor Laboratory Press, Cold Spring Harbor, N.Y.). Using knowledge of the genetic code in combination with the polypeptide sequences described herein, sets of degenerate oligonucleotides can be prepared. Such oligonucleotides are useful as primers, for example, in polymerase chain reactions (PCR), whereby DNA fragments are isolated and amplified. In certain embodiments, degenerate primers can be used as probes for genetic libraries. Such libraries include cDNA libraries, genomic libraries, and even electronic express sequence tag or DNA libraries. Homologous sequences identified by this method would then be used as probes to identify homologues of the sequences identified herein.

Also of potential use are polynucleotides and oligonucleotides (for example, primers or probes) that hybridize under decreased stringency conditions, typically moderately stringent conditions, and commonly highly stringent conditions to the polynucleotide(s), as described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y. and can be readily determined by those having ordinary skill in the art based on, for example, the length or base composition of the polynucleotide.

One way of achieving moderately and high stringent conditions are defined herein. It should be understood that the wash temperature and wash salt concentration can be adjusted as necessary to achieve a desired degree of stringency by applying the basic principles that govern hybridization reactions and duplex stability, as known to those skilled in the art and described further below (see, for example, Sambrook, J., E. F. Fritsch, and T. Maniatis (1989, Molecular Cloning: A Laboratory Manual, Cold Spring Harbor Laboratory Press, Cold Spring Harbor, N.Y). When hybridizing a polynucleotide to a polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. The hybridization temperature for hybrids anticipated to be less than 50 base pairs in length should be 5 to 10° C. less than the melting temperature of the hybrid, where melting temperature is determined according to the following equations. For hybrids less than 18 base pairs in length, melting temperature (C)=2 (number of A+T bases)+4 (number of G+C bases). For hybrids above 18 base pairs in length, melting temperature (° C.)=81.5+16.6 (log 10 [Na+])+0.41 (% G+C)−(600/N), where N is the number of bases in the hybrid, and [Na+] is the concentration of sodium ions in the hybridization buffer ([Na+] for 1× Standard Sodium Citrate=0.165M). Typically, each such hybridizing polynucleotide has a length that is at least 25% (commonly at least 50%, 60%, or 70%, and most commonly at least 80%) of the length of a polynucleotide to which it hybridizes, and has at least 60% sequence identity (for example, at least 70%, 75%, 80%, 85%, 90%, 95%, 96%, 97%, 98%, 99% or 100%) with a polynucleotide to which it hybridizes.

As will be understood by the person skilled in the art, a linear DNA has two possible orientations: the 5′-to-3′ direction and the 3′-to-5′ direction. For example, if a first sequence is positioned in the 5′-to-3′ direction, and if a second sequence is positioned in the 5′-to-3′ direction within the same polynucleotide molecule/strand, then the first sequence and the second sequence are orientated in the same direction, or have the same orientation. Typically, a promoter sequence and a gene of interest under the regulation of the given promoter are positioned in the same orientation. However, with respect to the first sequence positioned in the 5′-to-3′ direction, if a second sequence is positioned in the 3′-to-5′ direction within the same polynucleotide molecule/strand, then the first sequence and the second sequence are orientated in anti-sense direction, or have anti-sense orientation. Two sequences having anti-sense orientations with respect to each other can be alternatively described as having the same orientation, if the first sequence (5′-to-3′ direction) and the reverse complementary sequence of the first sequence (first sequence positioned in the 5′-to-3′) are positioned within the same polynucleotide molecule/strand. The sequences set forth herein are shown in the 5′-to-3′ direction.

In another aspect, there is provided an isolated polypeptide comprising, consisting or consisting essentially of a polypeptide having at least 60% sequence identity to any of the polypeptide described herein, including any of the polypeptides shown in the sequence listing. Suitably, the isolated polypeptide comprises, consists or consists essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity thereto.

In one embodiment, there is provided a polypeptide encoded by SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:25 or SEQ ID NO:27.

In another embodiment, there is provided an isolated polypeptide comprising, consisting or consisting essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO: 2, 4, 6, 8, 10, 12, 14, 16, 18, 20, 25 or 27.

The polypeptide can include sequences comprising a sufficient or substantial degree of identity or similarity to SEQ ID NO: 2, 4, 6, 8, 10, 12, 14, 16, 18, 20, 25 or 27 to function as nicotinamidase. The fragments of the polypeptide(s) typically retain some or all of the function or activity of the full length sequence.

As discussed herein, the polypeptides also include mutants produced by introducing any type of alterations (for example, insertions, deletions, or substitutions of amino acids; changes in glycosylation states; changes that affect refolding or isomerizations, three-dimensional structures, or self-association states), which can be deliberately engineered or isolated naturally provided that they still have some or all of their function or activity. Suitably, this function or activity is modulated.

A deletion refers to removal of one or more amino acids from a polypeptide. An insertion refers to one or more amino acid residues being introduced into a predetermined site in a polypeptide. Insertions may comprise intra-sequence insertions of single or multiple amino acids. A substitution refers to the replacement of amino acids of the polypeptide with other amino acids having similar properties (such as similar hydrophobicity, hydrophilicity, antigenicity, propensity to form or break a-helical structures or β-sheet structures). Amino acid substitutions are typically of single residues, but may be clustered depending upon functional constraints placed upon the polypeptide and may range from about 1 to about 10 amino acids. The amino acid substitutions are suitably conservative amino acid substitutions as described below. Amino acid substitutions, deletions and/or insertions can be made using peptide synthetic techniques—such as solid phase peptide synthesis or by recombinant DNA manipulation. Methods for the manipulation of DNA sequences to produce substitution, insertion or deletion variants of a polypeptide are well known in the art. The variant may have alterations which produce a silent change and result in a functionally equivalent polypeptide. Deliberate amino acid substitutions may be made on the basis of similarity in polarity, charge, solubility, hydrophobicity, hydrophilicity and the amphipathic nature of the residues as long as the secondary binding of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example according to the Table below. Amino acids in the same block in the second column and suitably in the same line in the third column may be substituted for each other:

| | | |
|---|---|---|
| ALIPHATIC | Non-polar | Gly Ala Pro |
| | | Ile Leu Val |
| | Polar-uncharged | Cys Ser Thr Met |
| | | Asn Gly |
| | Polar-charged | Asp Glu |
| | | Lys Arg |
| AROMATIC | | His Phe TrpTyr |

The polypeptide may be a mature polypeptide or an immature polypeptide or a polypeptide derived from an immature polypeptide. Polypeptides may be in linear form or cyclized using known methods. Polypeptides typically comprise at least 10, at least 20, at least 30, or at least 40 contiguous amino acids.

Recombinant constructs can be used to transform plants or plant cells in order to modulate polypeptide expression, function or activity. A recombinant polynucleotide construct can comprise a polynucleotide encoding one or more polynucleotides as described herein, operably linked to a regulatory region suitable for expressing the polypeptide. Thus, a polynucleotide can comprise a coding sequence that encodes the polypeptide as described herein. Plants or plant cells in which polypeptide expression, function or activity are modulated can include mutant, non-naturally occurring, transgenic, man-made or genetically engineered plants or plant cells. Suitably, the transgenic plant or plant cell comprises a genome that has been altered by the stable integration of recombinant DNA. Recombinant DNA includes DNA which has been genetically engineered and constructed outside of a cell and includes DNA containing naturally occurring DNA or cDNA or synthetic DNA. A transgenic plant can include a plant regenerated from an originally-transformed plant cell and progeny transgenic plants from later generations or crosses of a transformed plant. Suitably, the transgenic modification alters the expression or function or activity of the polynucleotide or the polypeptide described herein as compared to a control plant.

The polypeptide encoded by a recombinant polynucleotide can be a native polypeptide, or can be heterologous to the cell. In some cases, the recombinant construct contains a polynucleotide that modulates expression, operably linked to a regulatory region. Examples of suitable regulatory regions are described herein.

Vectors containing recombinant polynucleotide constructs such as those described herein are also provided. Suitable vector backbones include, for example, those routinely used in the art such as plasmids, viruses, artificial chromosomes, bacterial artificial chromosomes, yeast artificial chromosomes, or bacteriophage artificial chromosomes. Suitable expression vectors include, without limitation, plasmids and viral vectors derived from, for example, bacteriophage, baculoviruses, and retroviruses. Numerous vectors and expression systems are commercially available.

The vectors can include, for example, origins of replication, scaffold attachment regions or markers. A marker gene can confer a selectable phenotype on a plant cell. For example, a marker can confer biocide resistance, such as resistance to an antibiotic (for example, kanamycin, G418, bleomycin, or hygromycin), or an herbicide (for example, glyphosate, chlorsulfuron or phosphinothricin). In addition, an expression vector can include a tag sequence designed to facilitate manipulation or detection (for example, purification or localization) of the expressed polypeptide. Tag sequences, such as luciferase, beta-glucuronidase, green fluorescent polypeptide, glutathione S-transferase, polyhistidine, c-myc or hemagglutinin sequences typically are expressed as a fusion with the encoded polypeptide. Such tags can be inserted anywhere within the polypeptide, including at either the carboxyl or amino terminus.

A plant or plant cell can be transformed by having the recombinant polynucleotide integrated into its genome to become stably transformed. The plant or plant cell described herein can be stably transformed. Stably transformed cells typically retain the introduced polynucleotide with each cell division. A plant or plant cell can be transiently transformed such that the recombinant polynucleotide is not integrated into its genome. Transiently transformed cells typically lose all or some portion of the introduced recombinant polynucleotide with each cell division such that the introduced recombinant polynucleotide cannot be detected in daughter cells after a sufficient number of cell divisions.

A number of methods are available in the art for transforming a plant cell including biolistics, gene gun techniques, Agrobacterium-mediated transformation, viral vector-mediated transformation, freeze-thaw method, microparticle bombardment, direct DNA uptake, sonication, microinjection, plant virus-mediated transfer, and electroporation. The Agrobacterium system for integration of foreign DNA into plant chromosomes has been extensively studied, modified, and exploited for plant genetic engineering. Naked recombinant DNA molecules comprising DNA sequences corresponding to the subject purified polypeptide operably linked, in the sense or antisense orientation, to regulatory sequences are joined to appropriate T-DNA sequences by conventional methods. These are introduced into protoplasts by polyethylene glycol techniques or by electroporation techniques, both of which are standard. Alternatively, such vectors comprising recombinant DNA molecules encoding the subject purified polypeptide are introduced into live Agrobacterium cells, which then transfer the DNA into the plant cells. Transformation by naked DNA without accompanying T-DNA vector sequences can be accomplished via fusion of protoplasts with DNA-containing liposomes or via electroporation. Naked DNA unaccompanied by T-DNA vector sequences can also be used to transform cells via inert, high velocity microprojectiles.

If a cell or cultured tissue is used as the recipient tissue for transformation, plants can be regenerated from transformed cultures if desired, by techniques known to those skilled in the art.

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a polynucleotide can be modulated in a similar manner. Some suitable regulatory regions initiate transcription only, or predominantly, in certain cell types. Methods for identifying and characterizing regulatory regions in plant genomic DNA are known in the art.

Suitable promoters include tissue-specific promoters recognized by tissue-specific factors present in different tissues or cell types (for example, root-specific promoters, shoot-specific promoters, xylem-specific promoters), or present during different developmental stages, or present in response to different environmental conditions. Suitable promoters include constitutive promoters that can be activated in most cell types without requiring specific inducers. Examples of suitable promoters for controlling RNAi polypeptide production include the cauliflower mosaic virus 35S (CaMV/35S), SSU, OCS, lib4, usp, STLS1, B33, nos or ubiquitin- or phaseolin-promoters. Persons skilled in the art are capable of generating multiple variations of recombinant promoters.

Tissue-specific promoters are transcriptional control elements that are only active in particular cells or tissues at specific times during plant development, such as in vegetative tissues or reproductive tissues. Tissue-specific expression can be advantageous, for example, when the expression of polynucleotides in certain tissues is preferred. Examples of tissue-specific promoters under developmental control include promoters that can initiate transcription only (or primarily only) in certain tissues, such as vegetative tissues, for example, roots or leaves, or reproductive tissues, such as fruit, ovules, seeds, pollen, pistols, flowers, or any embryonic tissue. Reproductive tissue-specific promoters may be, for example, anther-specific, ovule-specific, embryo-specific, endosperm-specific, integument-specific, seed and seed coat-specific, pollen-specific, petal-specific, sepal-specific, or combinations thereof.

Suitable leaf-specific promoters include pyruvate, orthophosphate dikinase (PPDK) promoter from C4 plant (maize), cab-m1Ca+2 promoter from maize, the Arabidopsis thaliana myb-related gene promoter (Atmyb5), the ribulose biphosphate carboxylase (RBCS) promoters (for example, the tomato RBCS 1, RBCS2 and RBCS3A genes expressed in leaves and light-grown seedlings, RBCS1 and RBCS2 expressed in developing tomato fruits or ribulose bisphosphate carboxylase promoter expressed almost exclusively in mesophyll cells in leaf blades and leaf sheaths at high levels).

Suitable senescence-specific promoters include a tomato promoter active during fruit ripening, senescence and abscission of leaves, a maize promoter of gene encoding a cysteine protease, the promoter of 82E4 and the promoter of SAG genes. Suitable anther-specific promoters can be used. Suitable root-preferred promoters known to persons skilled in the art may be selected. Suitable seed-preferred promoters include both seed-specific promoters (those promoters active during seed development such as promoters of seed storage polypeptides) and seed-germinating promoters (those promoters active during seed germination). Such seed-preferred promoters include Cim1 (cytokinin-induced message); cZ19B1 (maize 19 kDa zein); milps (myo-inositol-1-phosphate synthase); mZE40-2, also known as Zm-40; nuclc; and celA (cellulose synthase). Gama-zein is an endosperm-specific promoter. Glob-1 is an embryo-specific promoter. For dicots, seed-specific promoters include bean beta-phaseolin, napin, β-conglycinin, soybean lectin, cruciferin, and the like. For monocots, seed-specific promoters include a maize 15 kDa zein promoter, a 22 kDa zein promoter, a 27 kDa zein promoter, a g-zein promoter, a 27 kDa gamma-zein promoter (such as gzw64A promoter, see Genbank Accession number S78780), a waxy promoter, a shrunken 1 promoter, a shrunken 2 promoter, a globulin 1 promoter (see Genbank Accession number L22344), an Itp2 promoter, cim1 promoter, maize end1 and end2 promoters, nuc1 promoter, Zm40 promoter, eep1 and eep2; lec1, thioredoxin H promoter; mlip15 promoter, PCNA2 promoter; and the shrunken-2 promoter.

Examples of inducible promoters include promoters responsive to pathogen attack, anaerobic conditions, elevated temperature, light, drought, cold temperature, or high salt concentration. Pathogen-inducible promoters include those from pathogenesis-related polypeptides (PR polypeptides), which are induced following infection by a pathogen (for example, PR polypeptides, SAR polypeptides, beta-1,3-glucanase, chitinase).

In addition to plant promoters, other suitable promoters may be derived from bacterial origin for example, the octopine synthase promoter, the nopaline synthase promoter and other promoters derived from Ti plasmids, or may be derived from viral promoters (for example, 35S and 19S RNA promoters of cauliflower mosaic virus (CaMV), constitutive promoters of tobacco mosaic virus, cauliflower mosaic virus (CaMV) 19S and 35S promoters, or figwort mosaic virus 35S promoter).

Suitable methods of introducing polynucleotides into plant cells and subsequent insertion into the plant genome include microinjection (Crossway et al., Biotechniques 4:320-334 (1986)), electroporation (Riggs et al., Proc. Natl. Acad. Sci. USA 83:5602-5606 (1986)), Agrobacterium-mediated transformation (U.S. Pat. Nos. 5,981,840 and 5,563,055), direct gene transfer (Paszkowski et al., EMBO J. 3:2717-2722 (1984)), and ballistic particle acceleration (see, for example, U.S. Pat. Nos. 4,945,050; 5,879,918; 5,886,244; 5,932,782; Tomes et al., in Plant Cell, Tissue, and Organ Culture: Fundamental Methods, ed. Gamborg and Phillips (Springer-Verlag, Berlin) (1995); and McCabe et al., Biotechnology 6:923-926 (1988)).

A plant or plant cell comprising a mutation in one or more polynucleotides or polypeptides described herein is disclosed, wherein said mutation results in modulated function or activity. Aside from the mutations described, the mutant plants or plant cells can have one or more further mutations either in the same polynucleotides or polypeptides as described herein or in one or more other polynucleotides or polypeptides within the genome.

There is also provided a method for modulating the level of a polypeptide in a (cured) plant or in (cured) plant material said method comprising introducing into the genome of said plant one or more mutations that modulate expression of at least one gene, wherein said at least one gene is selected from the sequences according to the present disclosure.

There is also provided a method for identifying a plant with modulated levels of nicotine, said method comprising screening a polynucleotide sample from a plant of interest for the presence of one or more mutations in the sequences according to the present disclosure, and optionally correlating the identified mutation(s) with mutation(s) that are known to modulate levels of nicotine.

There is also disclosed a plant or plant cell that is heterozygous or homozygous for one or more mutations in a gene according to the present disclosure, wherein said mutation results in modulated expression of the gene or function or activity of the polypeptide encoded thereby. A number of approaches can be used to combine mutations in one plant including sexual crossing. A plant having one or more favourable heterozygous or homozygous mutations in a gene according to the present disclosure that modulates expression of the gene or the function or activity of the polypeptide encoded thereby can be crossed with a plant having one or more favourable heterozygous or homozygous mutations in one or more other genes that modulate expression thereof or the function or activity of the polypeptide encoded thereby. In one embodiment, crosses are made in order to introduce one or more favourable heterozygous or homozygous mutations within gene according to the present disclosure within the same plant. The function or activity of one or more polypeptides of the present disclosure in a plant is increased or decreased if the function or activity is lower or higher than the function or activity of the same polypeptide(s) in a plant that has not been modified to inhibit the function or activity of that polypeptide and which has been cultured, harvested and cured using the same protocols.

In some embodiments, the mutation(s) is introduced into a plant or plant cell using a mutagenesis approach, and the introduced mutation is identified or selected using methods known to those of skill in the art—such as Southern blot analysis, DNA sequencing, PCR analysis, or phenotypic analysis. Mutations that impact gene expression or that interfere with the function of the encoded polypeptide can be determined using methods that are well known in the art. Insertional mutations in gene exons usually result in null-mutants. Mutations in conserved residues can be particularly effective in inhibiting the metabolic function of the encoded polypeptide. It will be appreciated, for example, that a mutation in one or more of the highly conserved regions would likely alter polypeptide function, while a mutation outside of those highly conserved regions would likely have little to no effect on polypeptide function. In addition, a mutation in a single nucleotide can create a stop codon, which would result in a truncated polypeptide and, depending on the extent of truncation, loss of function.

Methods for obtaining mutant polynucleotides and polypeptides are also disclosed. Any plant of interest, including a plant cell or plant material can be genetically modified by various methods known to induce mutagenesis, including site-directed mutagenesis, oligonucleotide-directed mutagenesis, chemically-induced mutagenesis, irradiation-induced mutagenesis, mutagenesis utilizing modified bases, mutagenesis utilizing gapped duplex DNA, double-strand break mutagenesis, mutagenesis utilizing repair-deficient host strains, mutagenesis by total gene synthesis, DNA shuffling and other equivalent methods.

Fragments of polynucleotides and polypeptides are also disclosed. Fragments of a polynucleotide may encode polypeptide fragments that retain the biological function of the native polypeptide and hence are involved in the metabolite transport network in a plant. Alternatively, fragments of a polynucleotide that are useful as hybridization probes or PCR primers generally do not encode fragment polypeptides retaining biological function. Furthermore, fragments of the disclosed polynucleotides include those that can be assembled within recombinant constructs as discussed herein. Fragments of a polynucleotide may range from at least about 25 nucleotides, about 50 nucleotides, about 75 nucleotides, about 100 nucleotides about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1100 nucleotides, about 1200 nucleotides, about 1300 nucleotides or about 1400 nucleotides and up to the full-length polynucleotide encoding the polypeptides described herein. Fragments of a polypeptide may range from at least about 25 amino acids, about 50 amino acids, about 75 amino acids, about 100 amino acids about 150 amino acids, about 200 amino acids, about 250 amino acids, about 300 amino acids, about 400 amino acids, about 500 amino acids, and up to the full-length polypeptide described herein. Mutant polypeptide variants can be used to create mutant, non-naturally occurring or transgenic plants (for example, mutant, non-naturally occurring, transgenic, man-made or genetically engineered plants) or plant cells comprising one or more mutant polypeptide variants. Suitably, mutant polypeptide variants retain the function of the unmutated polypeptide. The function of the mutant polypeptide variant may be higher, lower or about the same as the unmutated polypeptide.

Mutations in the polynucleotides and polypeptides described herein can include man-made mutations or synthetic mutations or genetically engineered mutations. Mutations in the polynucleotides and polypeptides described herein can be mutations that are obtained or obtainable via a process which includes an in vitro or an in vivo manipulation step. Mutations in the polynucleotides and polypeptides described herein can be mutations that are obtained or obtainable via a process which includes intervention by man.

Methods that introduce a mutation randomly in a polynucleotide can include chemical mutagenesis and radiation mutagenesis. Chemical mutagenesis involves the use of exogenously added chemicals—such as mutagenic, teratogenic, or carcinogenic organic compounds—to induce mutations. Mutagens that create primarily point mutations and short deletions, insertions, missense mutations, simple sequence repeats, transversions, and/or transitions, including chemical mutagens or radiation, may be used to create the mutations. Mutagens include ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz (a) anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Spontaneous mutations in the locus that may not have been directly caused by the mutagen are also contemplated provided that they result in the desired phenotype. Suitable mutagenic agents can also include, for example, ionising radiation—such as X-rays, gamma rays, fast neutron irradiation and UV radiation. The dosage of the mutagenic chemical or radiation is determined experimentally for each type of plant tissue such that a mutation frequency is obtained that is below a threshold level characterized by lethality or reproductive sterility. Any method of plant polynucleotide preparation known to those of skill in the art may be used to prepare the plant polynucleotide for mutation screening.

The mutation process may include one or more plant crossing steps.

After mutation, screening can be performed to identify mutations that create premature stop codons or otherwise non-functional genes. After mutation, screening can be performed to identify mutations that create functional genes that are capable of being expressed at increased or decreased levels. Screening of mutants can be carried out by sequencing, or by the use of one or more probes or primers specific to the gene or polypeptide. Specific mutations in polynucleotides can also be created that can result in modulated gene expression, modulated stability of mRNA, or modulated stability of polypeptide. Such plants are referred to herein as “non-naturally occurring” or “mutant” plants. Typically, the mutant or non-naturally occurring plants will include at least a portion of foreign or synthetic or man-made nucleotide (for example, DNA or RNA) that was not present in the plant before it was manipulated. The foreign nucleotide may be a single nucleotide, two or more nucleotides, two or more contiguous nucleotides or two or more non-contiguous nucleotides—such as at least 10, 20, 30, 40, 50, 100, 200, 300, 400, 500, 600, 700, 800, 900, 1000, 1100, 1200, 1300, 1400 or 1500 or more contiguous or non-contiguous nucleotides.

Other than mutagenesis, compositions that can modulate the expression or function or activity of one or more of the polynucleotides or polypeptides described herein include sequence-specific polynucleotides that can interfere with the transcription of one or more endogenous gene(s); sequence-specific polynucleotides that can interfere with the translation of RNA transcripts (for example, double-stranded RNAs, siRNAs, ribozymes); sequence-specific polypeptides that can interfere with the stability of one or more polypeptides; sequence-specific polynucleotides that can interfere with the enzymatic function of one or more polypeptides or the binding function of one or more polypeptides with respect to substrates or regulatory polypeptides; antibodies that exhibit specificity for one or more polypeptides; small molecule compounds that can interfere with the stability of one or more polypeptides or the enzymatic function of one or more polypeptides or the binding function of one or more polypeptides; zinc finger polypeptides that bind one or more polynucleotides; and meganucleases that have function towards one or more polynucleotides. Gene editing technologies, genetic editing technologies and genome editing technologies are well known in the art.

Zinc finger polypeptides can be used to modulate the expression or function or activity of one or more of the polynucleotides described herein. In various embodiments, a genomic DNA sequence comprising a part of or all of the coding sequence of the polynucleotide is modified by zinc finger nuclease-mediated mutagenesis. The genomic DNA sequence is searched for a unique site for zinc finger polypeptide binding. Alternatively, the genomic DNA sequence is searched for two unique sites for zinc finger polypeptide binding wherein both sites are on opposite strands and close together, for example, 1, 2, 3, 4, 5, 6 or more base pairs apart. Accordingly, zinc finger polypeptides that bind to polynucleotides are provided.

A zinc finger polypeptide may be engineered to recognize a selected target site in a gene. A zinc finger polypeptide can comprise any combination of motifs derived from natural zinc finger DNA-binding domains and non-natural zinc finger DNA-binding domains by truncation or expansion or a process of site-directed mutagenesis coupled to a selection method such as, but not limited to, phage display selection, bacterial two-hybrid selection or bacterial one-hybrid selection. The term “non-natural zinc finger DNA-binding domain” refers to a zinc finger DNA-binding domain that binds a three-base pair sequence within the polynucleotide target and that does not occur in the cell or organism comprising the polynucleotide which is to be modified. Methods for the design of zinc finger polypeptide which binds specific polynucleotides which are unique to a target gene are known in the art.

In other embodiments, a zinc finger polypeptide may be selected to bind to a regulatory sequence of a polynucleotide. More specifically, the regulatory sequence may comprise a transcription initiation site, a start codon, a region of an exon, a boundary of an exon-intron, a terminator, or a stop codon. Accordingly, the disclosure provides a mutant, non-naturally occurring or transgenic plant or plant cells, produced by zinc finger nuclease-mediated mutagenesis in the vicinity of or within one or more polynucleotides described herein, and methods for making such a plant or plant cell by zinc finger nuclease-mediated mutagenesis. Methods for delivering zinc finger polypeptide and zinc finger nuclease to a plant are similar to those described below for delivery of meganuclease.

In another aspect, methods for producing mutant, non-naturally occurring or transgenic or otherwise genetically-modified plants using meganucleases, such as I-Crel, are described. Naturally occurring meganucleases as well as recombinant meganucleases can be used to specifically cause a double-stranded break at a single site or at relatively few sites in the genomic DNA of a plant to allow for the disruption of one or more polynucleotides described herein. The meganuclease may be an engineered meganuclease with altered DNA-recognition properties. Meganuclease polypeptides can be delivered into plant cells by a variety of different mechanisms known in the art.

The disclosure encompass the use of meganucleases to inactivate a polynucleotide(s) described herein (or any combination thereof as described herein) in a plant cell or plant. Particularly, the disclosure provides a method for inactivating a polynucleotide in a plant using a meganuclease comprising: a) providing a plant cell comprising a polynucleotide as described herein; (b) introducing a meganuclease or a construct encoding a meganuclease into said plant cell; and (c) allowing the meganuclease to substantially inactivate the polynucleotide(s) Meganucleases can be used to cleave meganuclease recognition sites within the coding regions of a polynucleotide. Such cleavage frequently results in the deletion of DNA at the meganuclease recognition site following mutagenic DNA repair by non-homologous end joining. Such mutations in the gene coding sequence are typically sufficient to inactivate the gene. This method to modify a plant cell involves, first, the delivery of a meganuclease expression cassette to a plant cell using a suitable transformation method. For highest efficiency, it is desirable to link the meganuclease expression cassette to a selectable marker and select for successfully transformed cells in the presence of a selection agent. This approach will result in the integration of the meganuclease expression cassette into the genome, however, which may not be desirable if the plant is likely to require regulatory approval. In such cases, the meganuclease expression cassette (and linked selectable marker gene) may be segregated away in subsequent plant generations using conventional breeding techniques.

Following delivery of the meganuclease expression cassette, plant cells are grown, initially, under conditions that are typical for the particular transformation procedure that was used. This may mean growing transformed cells on media at temperatures below 26° C., frequently in the dark. Such standard conditions can be used for a period of time, suitably 1-4 days, to allow the plant cell to recover from the transformation process. At any point following this initial recovery period, growth temperature may be raised to stimulate the function of the engineered meganuclease to cleave and mutate the meganuclease recognition site.

One method of gene editing involves the use of transcription activator-like effector nucleases (TALENs) which induce double-strand breaks which cells can respond to with repair mechanisms. NHEJ reconnects DNA from either side of a double-strand break where there is very little or no sequence overlap for annealing. This repair mechanism induces errors in the genome via insertion or deletion, or chromosomal rearrangement. Any such errors may render the gene products coded at that location non-functional. For certain applications, it may be desirable to precisely remove the polynucleotide from the genome of the plant. Such applications are possible using a pair of engineered meganucleases, each of which cleaves a meganuclease recognition site on either side of the intended deletion. TALENs that are able to recognize and bind to a gene and introduce a double-strand break into the genome can also be used. Thus, in another aspect, methods for producing mutant, non-naturally occurring or transgenic or otherwise genetically-modified plants as described herein using TAL Effector Nucleases are contemplated.

Another method of gene editing involves the use of the bacterial CRISPR/Cas system. Bacteria and archaea exhibit chromosomal elements called clustered regularly interspaced short palindromic repeats (CRISPR) that are part of an adaptive immune system that protects against invading viral and plasmid DNA. In Type II CRISPR systems, CRISPR RNAs (crRNAs) function with trans-activating crRNA (tracrRNA) and CRISPR-associated (Cas) polypeptides to introduce double-stranded breaks in target DNA. Target cleavage by Cas9 requires base-pairing between the crRNA and tracrRNA as well as base pairing between the crRNA and the target DNA. Target recognition is facilitated by the presence of a short motif called a protospacer-adjacent motif (PAM) that conforms to the sequence NGG. This system can be harnessed for genome editing. Cas9 is normally programmed by a dual RNA consisting of the crRNA and tracrRNA. However, the core components of these RNAs can be combined into a single hybrid ‘guide RNA’ for Cas9 targeting. The use of a noncoding RNA guide to target DNA for site-specific cleavage promises to be significantly more straightforward than existing technologies—such as TALENs. Using the CRISPR/Cas strategy, retargeting the nuclease complex only requires introduction of a new RNA sequence and there is no need to reengineer the specificity of polypeptide transcription factors. CRISPR/Cas technology was implemented in plants in the method of international application WO 2015/189693 A1, which discloses a viral-mediated genome editing platform that is broadly applicable across plant species. The RNA2 genome of the tobacco rattle virus (TRV) was engineered to carry and deliver guide RNA into Nicotiana benthamiana plants overexpressing Cas9 endonuclease. In the context of the present disclosure, a guide RNA may be derived from any of the sequences disclosed herein and the teaching of WO 2015/189693 A1 applied to edit the genome of a plant cell and obtain a desired mutant plant. The fast pace of the development of the technology has generated a great variety of protocols with broad applicability in plantae, which have been well catalogued in a number of recent scientific review articles (for example, Schiml et al. Plant Methods 2016 12:8; and Khatodia et al. Front Plant Sci. 2016; 7:506). A review of CRISPR/Cas systems with a particular focus on its application in plants is given by Bortesi and Fischer (Biotechnology Advances Volume 33, Issue 1, January-February 2015, Pages 41-52). Bortesi and Fischer also make comparisons between the CRISPR/Cas technology, zinc finger nucleases, and TALENs. More recent developments in the use of CRISPR/Cas for manipulating plant genomes are discussed by Liu et al. in Acta Pharmaceutica Sinica B (Volume 7, Issue 3, May 2017, Pages 292-302) and Curr. Op. in Plant Biol. 2017 36, 1-8. CRISPR/Cas9 plasmids for use in plants are listed in “addgene”, the non-profit plasmid repository (addgene.org), and CRISPR/Cas plasmids are commercially available.

Antisense technology is another well-known method that can be used to modulate the expression of a polypeptide. A polynucleotide of the gene to be repressed is cloned and operably linked to a regulatory region and a transcription termination sequence so that the antisense strand of RNA is transcribed. The recombinant construct is then transformed into a plant cell and the antisense strand of RNA is produced. The polynucleotide need not be the entire sequence of the gene to be repressed, but typically will be substantially complementary to at least a portion of the sense strand of the gene to be repressed.

A polynucleotide may be transcribed into a ribozyme, or catalytic RNA, that affects expression of an mRNA. Ribozymes can be designed to specifically pair with virtually any target RNA and cleave the phosphodiester backbone at a specific location, thereby functionally inactivating the target RNA. Heterologous polynucleotides can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ polynucleotide. The construction and production of hammerhead ribozymes is known in the art. Hammerhead ribozyme sequences can be embedded in a stable RNA such as a transfer RNA (tRNA) to increase cleavage efficiency in vivo.

In one embodiment, the sequence-specific polynucleotide that can interfere with the translation of RNA transcript(s) is interfering RNA. RNA interference or RNA silencing is an evolutionarily conserved process by which specific mRNAs can be targeted for enzymatic degradation. A double-stranded RNA (double-stranded RNA) is introduced or produced by a cell (for example, double-stranded RNA virus, or interfering RNA polynucleotides) to initiate the interfering RNA pathway. The double-stranded RNA can be converted into multiple small interfering RNA (siRNA) duplexes of 21-24 bp length by RNases III, which are double-stranded RNA-specific endonucleases. The siRNAs can be subsequently recognized by RNA-induced silencing complexes that promote the unwinding of siRNA through an ATP-dependent process. The unwound antisense strand of the siRNA guides the activated RNA-induced silencing complexes to the targeted mRNA comprising a sequence complementary to the siRNA anti-sense strand. The targeted mRNA and the anti-sense strand can form an A-form helix, and the major groove of the A-form helix can be recognized by the activated RNA-induced silencing complexes. The target mRNA can be cleaved by activated RNA-induced silencing complexes at a single site defined by the binding site of the 5′-end of the siRNA strand. The activated RNA-induced silencing complexes can be recycled to catalyze another cleavage event.

Interfering RNA expression vectors may comprise interfering RNA constructs encoding interfering RNA polynucleotides that exhibit RNA interference by reducing the expression level of mRNAs, pre-mRNAs, or related RNA variants. The expression vectors may comprise a promoter positioned upstream and operably-linked to an Interfering RNA construct, as further described herein. Interfering RNA expression vectors may comprise a suitable minimal core promoter, a Interfering RNA construct of interest, an upstream (5′) regulatory region, a downstream (3′) regulatory region, including transcription termination and polyadenylation signals, and other sequences known to persons skilled in the art, such as various selection markers.

The double-stranded RNA molecules may include siRNA molecules assembled from a single oligonucleotide in a stem-loop structure, wherein self-complementary sense and antisense regions of the siRNA molecule are linked by means of a polynucleotide based or non-polynucleotide-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating interfering RNA.

The use of small hairpin RNA molecules is also contemplated. They comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a double-stranded RNA molecule (optionally with additional processing steps that may result in addition or removal of one, two, three or more nucleotides from the 3′ end or the 5′ end of either or both strands). The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end or the 5′ end of either or both strands). The spacer sequence is typically an unrelated polynucleotide that is situated between two complementary polynucleotides regions which, when annealed into a double-stranded polynucleotide, comprise a small hairpin RNA. The spacer sequence generally comprises between about 3 and about 100 nucleotides.

Any RNA polynucleotide of interest can be produced by selecting a suitable sequence composition, loop size, and stem length for producing the hairpin duplex. A suitable range for designing stem lengths of a hairpin duplex, includes stem lengths of at least about 10, 11, 12, 13, 14, 15, 16, 17, 18, 19 or 20 nucleotides—such as about 14-30 nucleotides, about 30-50 nucleotides, about 50-100 nucleotides, about 100-150 nucleotides, about 150-200 nucleotides, about 200-300 nucleotides, about 300-400 nucleotides, about 400-500 nucleotides, about 500-600 nucleotides, and about 600-700 nucleotides. A suitable range for designing loop lengths of a hairpin duplex, includes loop lengths of about 4-25 nucleotides, about 25-50 nucleotides, or longer if the stem length of the hair duplex is substantial. In certain embodiments, a double-stranded RNA or ssRNA molecule is between about 15 and about 40 nucleotides in length. In another embodiment, the siRNA molecule is a double-stranded RNA or ssRNA molecule between about 15 and about 35 nucleotides in length. In another embodiment, the siRNA molecule is a double-stranded RNA or ssRNA molecule between about 17 and about 30 nucleotides in length. In another embodiment, the siRNA molecule is a double-stranded RNA or ssRNA molecule between about 19 and about 25 nucleotides in length. In another embodiment, the siRNA molecule is a double-stranded RNA or ssRNA molecule between about 21 to about 23 nucleotides in length. In certain embodiments, hairpin structures with duplexed regions longer than 21 nucleotides may promote effective siRNA-directed silencing, regardless of loop sequence and length. Exemplary sequences for RNA interference are described herein.

The target mRNA sequence is typically between about 14 to about 50 nucleotides in length. The target mRNA can, therefore, be scanned for regions between about 14 and about 50 nucleotides in length that suitably meet one or more of the following criteria: an A+T/G+C ratio of between about 2:1 and about 1:2; an AA dinucleotide or a CA dinucleotide at the 5′ end; a sequence of at least 10 consecutive nucleotides unique to the target mRNA (that is, the sequence is not present in other mRNA sequences from the same plant); and no “runs” of more than three consecutive guanine (G) nucleotides or more than three consecutive cytosine (C) nucleotides. These criteria can be assessed using various techniques known in the art, for example, computer programs such as BLAST can be used to search publicly available databases to determine whether the selected sequence is unique to the target mRNA. Alternatively, a sequence can be selected (and a siRNA sequence designed) using computer software available commercially (for example, OligoEngine, Target Finder and the siRNA Design Tool which are commercially available).

In one embodiment, target mRNA sequences are selected that are between about 14 and about 30 nucleotides in length that meet one or more of the above criteria. In another embodiment, sequences are selected that are between about 16 and about 30 nucleotides in length that meet one or more of the above criteria. In a further embodiment, sequences are selected that are between about 19 and about 30 nucleotides in length that meet one or more of the above criteria. In another embodiment, sequences are selected that are between about 19 and about 25 nucleotides in length that meet one or more of the above criteria.

In an exemplary embodiment, the siRNA molecules comprise a specific antisense sequence that is complementary to at least 12, 13, 14, 15, 16, 17, 18, 19, 20, 21, 22, 23, 24, 25, 26, 27, 28, 29, 30, or more contiguous nucleotides of any one of the polynucleotides described herein. The specific antisense sequence comprised by the siRNA molecule can be identical or substantially identical to the complement. In one embodiment, the specific antisense sequence comprised by the siRNA molecule is at least about 75%, 80%, 85%, 90%, 95%, 96%, 97%, 98%, 99% or 100% identical to the complement of the target mRNA sequence. Methods of determining sequence identity are known in the art and can be determined, for example, by using the BLASTN program of the University of Wisconsin Computer Group (GCG) software or provided on the NCBI website.

One method for inducing double stranded RNA-silencing in plants is transformation with a gene construct producing hairpin RNA (see Nature (2000) 407, 319-320). Such constructs comprise inverted regions of the target gene sequence, separated by an appropriate spacer. The insertion of a functional plant intron region as a spacer fragment additionally increases the efficiency of the gene silencing induction, due to generation of an intron spliced hairpin RNA (Plant J. (2001), 27, 581-590). Suitably, the stem length is about 50 nucleotides to about 1 kilobases in length. Methods for producing intron spliced hairpin RNA are well described in the art (see for example, Bioscience, Biotechnology, and Biochemistry (2008) 72, 2, 615-617). Interfering RNA molecules having a duplex or double-stranded structure, for example double-stranded RNA or small hairpin RNA, can have blunt ends, or can have 3′ or 5′ overhangs. As used herein, “overhang” refers to the unpaired nucleotide or nucleotides that protrude from a duplex structure when a 3′-terminus of one RNA strand extends beyond the 5′-terminus of the other strand (3′ overhang), or vice versa (5′ overhang). The nucleotides comprising the overhang can be ribonucleotides, deoxyribonucleotides or modified versions thereof. In one embodiment, at least one strand of the interfering RNA molecule has a 3′ overhang from about 1 to about 6 nucleotides in length. In other embodiments, the 3′ overhang is from about 1 to about 5 nucleotides, from about 1 to about 3 nucleotides and from about 2 to about 4 nucleotides in length.

When the interfering RNA molecule comprises a 3′ overhang at one end of the molecule, the other end can be blunt-ended or have also an overhang (5′ or 3′). When the interfering RNA molecule comprises an overhang at both ends of the molecule, the length of the overhangs may be the same or different. In one embodiment, the interfering RNA molecule comprises 3′ overhangs of about 1 to about 3 nucleotides on both ends of the molecule. In a further embodiment, the interfering RNA molecule is a double-stranded RNA having a 3′ overhang of 2 nucleotides at both ends of the molecule. In yet another embodiment, the nucleotides comprising the overhang of the interfering RNA are TT dinucleotides or UU dinucleotides.

The interfering RNA molecules can comprise one or more 5′ or 3′-cap structures. The term “cap structure” refers to a chemical modification incorporated at either terminus of an oligonucleotide, which protects the molecule from exonuclease degradation, and may also facilitate delivery or localisation within a cell.

Another modification applicable to interfering RNA molecules is the chemical linkage to the interfering RNA molecule of one or more moieties or conjugates which enhance the function, cellular distribution, cellular uptake, bioavailability or stability of the interfering RNA molecule. The polynucleotides may be synthesized or modified by methods well established in the art. Chemical modifications include 2′ modifications, introduction of non-natural bases, covalent attachment to a ligand, and replacement of phosphate linkages with thiophosphate linkages. In this embodiment, the integrity of the duplex structure is strengthened by at least one, and typically two, chemical linkages.

The nucleotides at one or both of the two single strands may be modified to modulate the activation of cellular enzymes, such as, for example, without limitation, certain nucleases. Techniques for reducing or inhibiting the activation of cellular enzymes are known in the art including, but not limited to, 2′-amino modifications, 2′-fluoro modifications, 2′-alkyl modifications, uncharged backbone modifications, morpholino modifications, 2′-O-methyl modifications, and phosphoramidate.

Ligands may be conjugated to an interfering RNA molecule, for example, to enhance its cellular absorption. In certain embodiments, a hydrophobic ligand is conjugated to the molecule to facilitate direct permeation of the cellular membrane. In certain instances, conjugation of a cationic ligand to oligonucleotides often results in improved resistance to nucleases.

“Targeted Induced Local Lesions In Genomes” (TILLING) is another mutagenesis technology that can be used to generate and/or identify polynucleotides encoding polypeptides with modified expression, function or activity. TILLING also allows selection of plants carrying such mutants. TILLING combines high-density mutagenesis with high-throughput screening methods. Methods for TILLING are well known in the art (see McCallum et al., (2000) Nat Biotechnol 18:455-457 and Stemple (2004) Nat Rev Genet 5 (2): 145-50).

Various embodiments are directed to expression vectors comprising one or more of the polynucleotides or interfering RNA constructs that comprise one or more polynucleotides described herein.

Various embodiments are directed to expression vectors comprising one or more of the polynucleotides or one or more interfering RNA constructs described herein.

Various embodiments are directed to expression vectors comprising one or more polynucleotides or one or more interfering RNA constructs encoding one or more interfering RNA polynucleotides described herein that are capable of self-annealing to form a hairpin structure, in which the construct comprises (a) one or more of the polynucleotides described herein; (b) a second sequence encoding a spacer element that forms a loop of the hairpin structure; and (c) a third sequence comprising a reverse complementary sequence of the first sequence, positioned in the same orientation as the first sequence, wherein the second sequence is positioned between the first sequence and the third sequence, and the second sequence is operably-linked to the first sequence and to the third sequence.

The disclosed sequences can be utilised for constructing various polynucleotides that do not form hairpin structures. For example, a double-stranded RNA can be formed by (1) transcribing a first strand of the DNA by operably-linking to a first promoter, and (2) transcribing the reverse complementary sequence of the first strand of the DNA fragment by operably-linking to a second promoter. Each strand of the polynucleotide can be transcribed from the same expression vector, or from different expression vectors. The RNA duplex having RNA interference can be enzymatically converted to siRNAs to modulate RNA levels.

Thus, various embodiments are directed to expression vectors comprising one or more polynucleotides or interfering RNA constructs described herein encoding interfering RNA polynucleotides capable of self-annealing, in which the construct comprises (a) one or more of the polynucleotides described herein; and (b) a second sequence comprising a complementary (for example, reverse complementary) sequence of the first sequence, positioned in the same orientation as the first sequence.

Various compositions and methods are provided for modulating the endogenous expression levels of one or more of the polypeptides described herein (or any combination thereof as described herein) by promoting co-suppression of gene expression.

Various compositions and methods are provided for modulating the endogenous gene expression level by modulating the translation of mRNA. A host (tobacco) plant cell can be transformed with an expression vector comprising: a promoter operably-linked to a polynucleotide, positioned in anti-sense orientation with respect to the promoter to enable the expression of RNA polynucleotides having a sequence complementary to a portion of mRNA. Various expression vectors for modulating the translation of mRNA may comprise: a promoter operably-linked to a polynucleotide in which the sequence is positioned in anti-sense orientation with respect to the promoter. The lengths of anti-sense RNA polynucleotides can vary, and may be from about 15-20 nucleotides, about 20-30 nucleotides, about 30-50 nucleotides, about 50-75 nucleotides, about 75-100 nucleotides, about 100-150 nucleotides, about 150-200 nucleotides, and about 200-300 nucleotides.

Alternatively, genes can be targeted for inactivation by introducing transposons (for example, IS elements) into the genomes of plants of interest. These mobile genetic elements can be introduced by sexual cross-fertilization and insertion mutants can be screened for loss in polypeptide function. The disrupted gene in a parent plant can be introduced into other plants by crossing the parent plant with plant not subjected to transposon-induced mutagenesis by, for example, sexual cross-fertilization. Any standard breeding techniques known to persons skilled in the art can be utilized. In one embodiment, one or more genes can be inactivated by the insertion of one or more transposons. Mutations can result in homozygous disruption of one or more genes, in heterozygous disruption of one or more genes, or a combination of both homozygous and heterozygous disruptions if more than one gene is disrupted. Suitable transposable elements include retrotransposons, retroposons, and SINE-like elements. Such methods are known to persons skilled in the art.

Alternatively, genes can be targeted for inactivation by introducing ribozymes derived from a number of small circular RNAs that are capable of self-cleavage and replication in plants. These RNAs can replicate either alone (viroid RNAs) or with a helper virus (satellite RNAs). Examples of suitable RNAs include those derived from avocado sunblotch viroid and satellite RNAs derived from tobacco ringspot virus, lucerne transient streak virus, velvet tobacco mottle virus, solanum nodiflorum mottle virus, and subterranean clover mottle virus. Various target RNA-specific ribozymes are known to persons skilled in the art.

The mutant or non-naturally occurring plants or plant cells can have any combination of one or more mutations in one or more genes which results in modulated expression or function or activity of those genes or their products. For example, the mutant or non-naturally occurring plants or plant cells may have a single mutation in a single gene; multiple mutations in a single gene; a single mutation in two or more or three or more or four or more genes; or multiple mutations in two or more or three or more or four or more genes. Examples of such mutations are described herein. By way of further example, the mutant or non-naturally occurring plants or plant cells may have one or more mutations in a specific portion of the gene(s)—such as in a region of the gene that encodes an active site of the polypeptide or a portion thereof. By way of further example, the mutant or non-naturally occurring plants or plant cells may have one or more mutations in a region outside of one or more gene(s)—such as in a region upstream or downstream of the gene it regulates provided that they modulate the function or expression of the gene(s). Upstream elements can include promoters, enhancers or transcription factors. Some elements—such as enhancers—can be positioned upstream or downstream of the gene it regulates. The element(s) need not be located near to the gene that it regulates since some elements have been found located several hundred thousand base pairs upstream or downstream of the gene that it regulates. The mutant or non-naturally occurring plants or plant cells may have one or more mutations located within the first 100 nucleotides of the gene(s), within the first 200 nucleotides of the gene(s), within the first 300 nucleotides of the gene(s), within the first 400 nucleotides of the gene(s), within the first 500 nucleotides of the gene(s), within the first 600 nucleotides of the gene(s), within the first 700 nucleotides of the gene(s), within the first 800 nucleotides of the gene(s), within the first 900 nucleotides of the gene(s), within the first 1000 nucleotides of the gene(s), within the first 1100 nucleotides of the gene(s), within the first 1200 nucleotides of the gene(s), within the first 1300 nucleotides of the gene(s), within the first 1400 nucleotides of the gene(s) or within the first 1500 nucleotides of the gene(s). The mutant or non-naturally occurring plants or plant cells may have one or more mutations located within the first, second, third, fourth, fifth, sixth, seventh, eighth, ninth, tenth, eleventh, twelfth, thirteenth, fourteenth or fifteenth set of 100 nucleotides of the gene(s) or combinations thereof. Mutant or non-naturally occurring plants or plant cells (for example, mutant, non-naturally occurring or transgenic plants or plant cells and the like, as described herein) comprising the mutant polypeptide variants are disclosed. In one embodiment, seeds from plants are mutagenised and then grown into first generation mutant plants. The first generation plants are then allowed to self-pollinate and seeds from the first generation plant are grown into second generation plants, which are then screened for mutations in their loci. Though the mutagenized plant material can be screened for mutations, an advantage of screening the second generation plants is that all somatic mutations correspond to germline mutations. One of skill in the art would understand that a variety of plant materials, including but not limited to, seeds, pollen, plant tissue or plant cells, may be mutagenised in order to create the mutant plants. However, the type of plant material mutagenised may affect when the plant polynucleotide is screened for mutations. For example, when pollen is subjected to mutagenesis prior to pollination of a non-mutagenized plant the seeds resulting from that pollination are grown into first generation plants. Every cell of the first generation plants will contain mutations created in the pollen; thus these first generation plants may then be screened for mutations instead of waiting until the second generation.

Plant heath, also referred to as fitness, may be measured by proxy using plant biomass. Plant biomass is the weight of a plant, suitably the dried weight or the fresh weight. It may be the whole weight of the plant or part of a plant. Suitably the plant biomass measured is that of the fresh green leaves (also referred to herein as “fresh leaf biomass” and “green leaf biomass”). Suitably, the biomass of a plant is compared to the biomass of a control plant grown under the same conditions.

The mutant or non-naturally occurring plants or plant cells may have a biomass the same as control plants or plant cells. The mutant or non-naturally occurring plants or plant cells may have a biomass reduced by at least 2%, 5%, 8%, 10%, 12%, 15%, 20%, 25% or 30% compared to the biomass of a control plants or plant cells grown under the same conditions.

The mutant or non-naturally occurring plants or plant cells may have a biomass increased by at least 2%, 5%, 8%, 10%, 12%, 15%, 20%, 25% or 30% compared to the biomass of a control plants or plant cells grown under the same conditions. A small effect on plant biomass, comprising an increase or decrease of 15% or less, is unlikely to affect plant health or fitness overall.

Plant health or fitness may also be measured by seed production. Seed production includes the number and biomass of seeds. The mutant or non-naturally occurring plants may have seed number or seed biomass reduced by at least 2%, 5%, 8%, 10%, 12%, 15%, 20%, 25% or 30% compared to the seed number or biomass of a control plants grown under the same conditions. The mutant or non-naturally occurring plants may have a seed number or seed biomass increased by at least 2%, 5%, 8%, 10%, 12%, 15%, 20%, 25% or 30 compared to the seed number or seed biomass of a control plants grown under the same conditions

Plant health or fitness may also be measured by general aspect. General aspect includes for example plant phenotype such as height, colour (as an indication of chlorophyll level), flowering time, plant architecture and leaf morphology.

Prepared polynucleotide from individual plants, plant cells, or plant material can optionally be pooled in order to expedite screening for mutations in the population of plants originating from the mutagenized plant tissue, cells or material. One or more subsequent generations of plants, plant cells or plant material can be screened. The size of the optionally pooled group is dependent upon the sensitivity of the screening method used.

After the samples are optionally pooled, they can be subjected to polynucleotide-specific amplification techniques, such as PCR. Any one or more primers or probes specific to the gene or the sequences immediately adjacent to the gene may be utilized to amplify the sequences within the optionally pooled sample. Suitably, the one or more primers or probes are designed to amplify the regions of the locus where useful mutations are most likely to arise. Most suitably, the primer is designed to detect mutations within regions of the polynucleotide. Additionally, it is preferable for the primer(s) and probe(s) to avoid known polymorphic sites in order to ease screening for point mutations. To facilitate detection of amplification products, the one or more primers or probes may be labelled using any conventional labelling method. Primer(s) or probe(s) can be designed based upon the sequences described herein using methods that are well understood in the art.

To facilitate detection of amplification products, the primer(s) or probe(s) may be labelled using any conventional labelling method. These can be designed based upon the sequences described herein using methods that are well understood in the art.

Polymorphisms may be identified by means known in the art and some have been described in the literature.

In some embodiments, a plant may be regenerated or grown from the plant, plant tissue or plant cell. Any suitable methods for regenerating or growing a plant from a plant cell or plant tissue may be used, such as, without limitation, tissue culture or regeneration from protoplasts. Suitably, plants may be regenerated by growing transformed plant cells on callus induction media, shoot induction media and/or root induction media. See, for example, McCormick et al., Plant Cell Reports 5:81-84 (1986). These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. Thus as used herein, “transformed seeds” refers to seeds that contain the nucleotide construct stably integrated into the plant genome.

Accordingly, in a further aspect there is provided a method of preparing a mutant plant. The method involves providing at least one cell of a plant comprising a gene encoding a functional polynucleotide described herein (or any combination thereof as described herein). Next, the at least one cell of the plant is treated under conditions effective to modulate the function of the polynucleotide(s) described herein. The at least one mutant plant cell is then propagated into a mutant plant, where the mutant plant has a modulated level of polypeptide(s) described (or any combination thereof as described herein) as compared to that of a control plant. In one embodiment of this method of making a mutant plant, the treating step involves subjecting the at least one cell to a chemical mutagenising agent as described above and under conditions effective to yield at least one mutant plant cell. In another embodiment of this method, the treating step involves subjecting the at least one cell to a radiation source under conditions effective to yield at least one mutant plant cell. The term “mutant plant” includes mutant plants in which the genotype is modified as compared to a control plant, suitably by means other than genetic engineering or genetic modification.

In certain embodiments, the mutant plant, mutant plant cell or mutant plant material may comprise one or more mutations that have occurred naturally in another plant, plant cell or plant material and confer a desired trait. This mutation can be incorporated (for example, introgressed) into another plant, plant cell or plant material (for example, a plant, plant cell or plant material with a different genetic background to the plant from which the mutation was derived) to confer the trait thereto. Thus, by way of example, a mutation that occurred naturally in a first plant may be introduced into a second plant—such as a second plant with a different genetic background to the first plant. The skilled person is therefore able to search for and identify a plant carrying naturally in its genome one or more mutant alleles of the genes described herein which confer a desired trait. The mutant allele(s) that occurs naturally can be transferred to the second plant by various methods including breeding, backcrossing and introgression to produce a lines, varieties or hybrids that have one or more mutations in the genes described herein. The same technique can also be applied to the introgression of one or more non-naturally occurring mutation(s) from a first plant into a second plant. Plants showing a desired trait may be screened out of a pool of mutant plants. Suitably, the selection is carried out utilising the knowledge of the polynucleotide as described herein. Consequently, it is possible to screen for a genetic trait as compared to a control. Such a screening approach may involve the application of conventional amplification and/or hybridization techniques as discussed herein. Thus, a further aspect of the present disclosure relates to a method for identifying a mutant plant comprising the steps of: (a) providing a sample comprising polynucleotide from a plant; and (b) determining the sequence of the polynucleotide, wherein a difference in the sequence of the polynucleotide as compared to the polynucleotide of a control plant is indicative that said plant is a mutant plant. In another aspect there is provided a method for identifying a mutant plant which accumulates increased or decreased levels of nicotine as compared to a control plant comprising the steps of: (a) providing a sample from a plant to be screened; (b) determining if said sample comprises one or more mutations in one or more of the polynucleotides described herein; and (c) determining the level of nicotine of said plant. Suitably the level of nicotine is determined in green leaves. In another aspect there is provided a method for preparing a mutant plant which has increased or decreased levels of nicotine as compared to a control plant comprising the steps of: (a) providing a sample from a first plant; (b) determining if said sample comprises one or more mutations in one or more the polynucleotides described herein that result in modulated levels of nicotine; and (c) transferring the one or more mutations into a second plant. Suitably the level of nicotine is determined in green leaves. The mutation(s) can be transferred into the second plant using various methods that are known in the art—such as by genetic engineering, genetic manipulation, introgression, plant breeding, backcrossing and the like. In one embodiment, the first plant is a naturally occurring plant. In one embodiment, the second plant has a different genetic background to the first plant. In another aspect there is provided a method for preparing a mutant plant which has increased or decreased levels of nicotine as compared to a control plant comprising the steps of: (a) providing a sample from a first plant; (b) determining if said sample comprises one or more mutations in one or more of the polynucleotides described herein that results in modulated levels of nicotine; and (c) introgressing the one or more mutations from the first plant into a second plant. Suitably the level of nicotine is determined in green leaves. In one embodiment, the step of introgressing comprises plant breeding, optionally including backcrossing and the like. In one embodiment, the first plant is a naturally occurring plant. In one embodiment, the second plant has a different genetic background to the first plant. In one embodiment, the first plant is not a cultivar or an elite cultivar. In one embodiment, the second plant is a cultivar or an elite cultivar. A further aspect relates to a mutant plant (including a cultivar or elite cultivar mutant plant) obtained or obtainable by the methods described herein. In certain embodiments, the “mutant plants” may have one or more mutations localised only to a specific region of the plant—such as within the sequence of the one or more polynucleotide(s) described herein. According to this embodiment, the remaining genomic sequence of the mutant plant will be the same or substantially the same as the plant prior to the mutagenesis.

In certain embodiments, the mutant plants may have one or more mutations localised in more than one genomic region of the plant—such as within the sequence of one or more of the polynucleotides described herein and in one or more further regions of the genome. According to this embodiment, the remaining genomic sequence of the mutant plant will not be the same or will not be substantially the same as the plant prior to the mutagenesis. In certain embodiments, the mutant plants may not have one or more mutations in one or more, two or more, three or more, four or more or five or more exons of the polynucleotide(s) described herein; or may not have one or more mutations in one or more, two or more, three or more, four or more or five or more introns of the polynucleotide(s) described herein; or may not have one or more mutations in a promoter of the polynucleotide(s) described herein; or may not have one or more mutations in the 3′ untranslated region of the polynucleotide(s) described herein; or may not have one or more mutations in the 5′ untranslated region of the polynucleotide(s) described herein; or may not have one or more mutations in the coding region of the polynucleotide(s) described herein; or may not have one or more mutations in the non-coding region of the polynucleotide(s) described herein; or any combination of two or more, three or more, four or more, five or more; or six or more thereof parts thereof.

In a further aspect there is provided a method of identifying a plant, a plant cell or plant material comprising a mutation in a gene encoding a polynucleotide described herein comprising: (a) subjecting a plant, a plant cell or plant material to mutagenesis; (b) obtaining a sample from said plant, plant cell or plant material or descendants thereof; and (c) determining the polynucleotide sequence of the gene or a variant or a fragment thereof, wherein a difference in said sequence is indicative of one or more mutations therein. This method also allows the selection of plants having mutation(s) that occur(s) in genomic regions that affect the expression of the gene in a plant cell, such as a transcription initiation site, a start codon, a region of an intron, a boundary of an exon-intron, a terminator, or a stop codon.

Plants suitable for use in genetic modification include monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae.

Suitable species may include members of the genera Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species may include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (tritic wheat times rye), bamboo, Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugarbeet), Manihot esculenta (cassaya), Lycopersicon esculentum (tomato), Lactuca sativa (lettuce), Musyclise alca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffeycliseca (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Rosa spp. (rose), Dianthus caryophyllus (carnation), Petunia spp. (petunia), Poinsettia pulcherrima (poinsettia), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy), Panicum virgatum (switchgrass), Sorghuycliseor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

Various embodiments are directed to mutant tobacco, non-naturally occurring tobacco or transgenic tobacco plants or plant cells modified to modulate gene expression levels thereby producing a plant or plant cell—such as a tobacco plant or plant cell—in which the expression level of a polypeptide is modulated within tissues of interest as compared to a control. The disclosed compositions and methods can be applied to any species of the genus Nicotiana, including N. rustica and N. tabacum (for example, LA B21, LN KY171, TI 1406, Basma, Galpao, Perique, Beinhart 1000-1, and Petico). Other species include N. acaulis, N. acuminata, N. africana, N. alata, N. ameghinoi, N. amplexicaulis, N. arentsii, N. attenuata, N. azambujae, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N. maritima, N. megalosiphon, N. miersii, N. noctiflora, N. nudicaulis, N. obtusifolia, N. occidentalis, N. occidentalis subsp. hesperis, N. otophora, N. paniculata, N. pauciflora, N. petunioides, N. plumbaginifolia, N. quadrivalvis, N. raimondii, N. repanda, N. rosulata, N. rosulata subsp. ingulba, N. rotundifolia, N. setchellii, N. simulans, N. solanifolia, N. spegazzinii, N. stocktonii, N. suaveolens, N. sylvestris, N. thyrsiflora, N. tomentosa, N. tomentosiformis, N. trigonophylla, N. umbratica, N. undulata, N. velutina, N. wigandioides, and N. x sanderae. In one embodiment, the plant is N. tabacum.

The use of tobacco cultivars and elite tobacco cultivars is also contemplated herein. The transgenic, non-naturally occurring or mutant plant may therefore be a tobacco variety or elite tobacco cultivar that comprises one or more transgenes, or one or more genetic mutations or a combination thereof. The genetic mutation(s) (for example, one or more polymorphisms) can be mutations that do not exist naturally in the individual tobacco variety or tobacco cultivar (for example, elite tobacco cultivar) or can be genetic mutation(s) that do occur naturally provided that the mutation does not occur naturally in the individual tobacco variety or tobacco cultivar (for example, elite tobacco cultivar).

Particularly useful Nicotiana tabacum varieties include Burley type, dark type, flue-cured type, and Oriental type tobaccos. Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF911, DT 538 LC Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KY14×L8 LC, Little Crittenden, McNair 373, McNair 944, msKY 14×L8, Narrow Leaf Madole, Narrow Leaf Madole LC, NBH 98, N-126, N-777LC, N-7371LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B13P, Xanthi (Mitchell-Mor), Bel-W3, 79-615, Samsun Holmes NN, KTRDC number 2 Hybrid 49, Burley 21, KY8959, KY9, MD 609, PG01, PG04, PO1, PO2, PO3, RG11, RG 8, VA509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpão Comum, HB04P, Hicks Broadleaf, Kabakulak Elassona, Kutsage E1, LA BU 21, NC 2326, NC 297, PVH 2110, Red Russian, Samsun, Saplak, Simmaba, Talgar 28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

Embodiments are also directed to compositions and methods for producing mutant plants, non-naturally occurring plants, hybrid plants, or transgenic plants that have been modified to modulate the expression or function of a polynucleotide(s) described herein (or any combination thereof as described herein). Advantageously, the mutant plants, non-naturally occurring plants, hybrid plants, or transgenic plants that are obtained may be similar or substantially the same in overall appearance to control plants. Various phenotypic characteristics such as degree of maturity, number of leaves per plant, stalk height, leaf insertion angle, leaf size (width and length), internode distance, and lamina-midrib ratio can be assessed by field observations.

One aspect relates to a seed of a mutant plant, a non-naturally occurring plant, a hybrid plant or a transgenic plant described herein. Suitably, the seed is a tobacco seed. A further aspect relates to pollen or an ovule of a mutant plant, a non-naturally occurring plant, a hybrid plant or a transgenic plant that is described herein. In addition, there is provided a mutant plant, a non-naturally occurring plant, a hybrid plant or a transgenic plant as described herein which further comprises a polynucleotide conferring male sterility.

Also provided is a tissue culture of regenerable cells of the mutant plant, non-naturally occurring plant, hybrid plant, or transgenic plant or a part thereof as described herein, which culture regenerates plants capable of expressing all the morphological and physiological characteristics of the parent. The regenerable cells include cells from leaves, pollen, embryos, cotyledons, hypocotyls, roots, root tips, anthers, flowers and a part thereof, ovules, shoots, stems, stalks, pith and capsules or callus or protoplasts derived therefrom.

One object is to provide mutant, transgenic or non-naturally occurring plants or parts thereof that exhibit modulated levels of nicotine in the plant material, for example, in cured leaves. Suitably, mutant, transgenic or non-naturally occurring plants or parts thereof that exhibit modulated levels of nicotine as compared to a control plant. Suitably, the mutant, transgenic or non-naturally occurring plants or parts thereof have substantially the same visual appearance as the control plant.

Accordingly, there is described herein mutant, transgenic or non-naturally occurring plants or parts thereof or plant cells that have modulated levels of nicotine compared to control cells or control plants. The mutant, transgenic or non-naturally occurring plants or plant cells have been modified to modulate the synthesis or function of one or more of the polypeptides described herein by modulating the expression of one or more of the corresponding polynucleotides described herein. Suitably, the modulated levels of nicotine are observed in at least the green leaves, suitably cured leaves. In certain embodiments, the level of nicotine in the plant—such as the green leaves, suitably cured leaves or cured tobacco—may be modulated. In certain embodiments, the level of nicotine in the plant—such as the green leaves, suitably cured leaves or cured tobacco—may be reduced.

A further aspect, relates to a mutant, non-naturally occurring or transgenic plant or cell, wherein the expression or the function of one or more of the polypeptides described herein is modulated and a part of the plant (for example, the green leaves, suitably cured leaves or cured tobacco) have decreased levels of nicotine of at least 5% therein as compared to a control plant in which the expression or the function of said polypeptide(s) has not been modulated. In certain embodiments, the level of nicotine in the plant—such as the green leaves, suitably cured leaves or cured tobacco—may be modulated, for example, by at least about 5%. Most suitably the level of nicotine in part of the plant is modulated by at least 10%, 15%, 20%, 25%, 30%, 25%, 40%, 45%, 50%, 55%, 60%, 65%, 70% or 75% as compared to a control plant. Most suitably the level of nicotine in part of the plant is reduced by at least 5%, 10%, 15%, 20%, 25%, 30%, 25%, 40%, 45%, 50%, 55%, 60%, 65%, 70% or 75% as compared to a control plant.

A still further aspect, relates to a cured plant material—such as cured leaf or cured tobacco—derived or derivable from a mutant, non-naturally occurring or transgenic plant or cell, wherein expression of one or more of the polynucleotides described herein or the function of the polypeptide encoded thereby is modulated and wherein the level of nicotine is modulated by at least 5% as compared to a control plant. Most suitably the level of nicotine in cured plant material is modulated by at least 10%, 15%, 20%, 25%, 30%, 25%, 40%, 45%, 50%, 55%, 60%, 65%, 70% or 75% as compared to a control plant. Most suitably the level of nicotine in cured plant material is reduced by at least 5%, 10%, 15%, 20%, 25%, 30%, 25%, 40%, 45%, 50%, 55%, 60%, 65%, 70% or 75% as compared to a control plant.

As total alkaloids are representative of more than 90% of the nicotine content of a tobacco plant, such as Nicotiana tabacum, the rest being mainly minor alkaloids anatabine, anabasine and nornicotine (see for example Jones et al., 2001; Journal of AOOAC INTERNATIONAL. (84) (2): 309-315), a measurement of the total alkaloids of a plant cell, a plant or part thereof may be considered a proxy for a measurement of the nicotine content of a plant cell, a plant or part thereof described herein and of a control a plant cell, a plant or part thereof described herein. Most suitably the total alkaloid in a plant, a part thereof or cured plant material is modulated by at least 10%, 15%, 20%, 25%, 30%, 25%, 40%, 45%, 50%, 55%, 60%, 65%, 70% or 75% as compared to a control plant. Most suitably the level of nicotine in a plant, part thereof or cured plant material is reduced by at least 5%, 10%, 15%, 20%, 25%, 30%, 25%, 40%, 45%, 50%, 55%, 60%, 65%, 70% or 75% as compared to a control plant.

Suitably the visual appearance of said plant or part thereof (for example, leaf) is substantially the same as the control plant. Suitably, the plant is a tobacco plant.

Embodiments are also directed to compositions and methods for producing mutant, non-naturally occurring or transgenic plants or plant cells that have been modified to modulate the expression or function of the one or more of the polynucleotides or polypeptides described herein which can result in plants or plant components (for example, leaves—such as green leaves or cured leaves—or tobacco) or plant cells with modulated nicotine content.

The mutant, non-naturally occurring or transgenic plants obtained according to this disclosure can be similar or substantially the same in visual appearance to the corresponding control plants. In one embodiment, the leaf weight of the mutant, non-naturally occurring or transgenic plant is substantially the same as the control plant. In one embodiment, the leaf number of the mutant, non-naturally occurring or transgenic plant is substantially the same as the control plant. In one embodiment, the leaf weight and the leaf number of the mutant, non-naturally occurring or transgenic plant is substantially the same as the control plant. In one embodiment, the stalk height of the mutant, non-naturally occurring or transgenic plants is substantially the same as the control plants at, for example, one, two or three or more months after field transplant or 10, 20, 30 or 36 or more days after topping. For example, the stalk height of the mutant, non-naturally occurring or transgenic plants is not less than the stalk height of the control plants. In another embodiment, the chlorophyll content of the mutant, non-naturally occurring or transgenic plants is substantially the same as the control plants. In another embodiment, the stalk height of the mutant, non-naturally occurring or transgenic plants is substantially the same as the control plants and the chlorophyll content of the mutant, non-naturally occurring or transgenic plants is substantially the same as the control plants. In other embodiments, the size or form or number or colouration of the leaves of the mutant, non-naturally occurring or transgenic plants is substantially the same as the control plants. Suitably, the plant is a tobacco plant.

In another aspect, there is provided a method for modulating the amount of nicotine in at least a part of a plant (for example, the leaves—such as cured leaves—or in tobacco), comprising the steps of: (i) modulating the expression or function of an one or more of the polypeptides described herein (or any combination thereof as described herein), suitably, wherein the polypeptide(s) is encoded by the corresponding polynucleotides described herein; (ii) measuring the level of nicotine in at least a part (for example, the leaves—such as cured leaves—or tobacco or in smoke) of the mutant, non-naturally occurring or transgenic plant obtained in step (i); and (iii) identifying a mutant, non-naturally occurring or transgenic plant in which the level of nicotine therein has been modulated in comparison to a control plant. Suitably, the visual appearance of said mutant, non-naturally occurring or transgenic plant is substantially the same as the control plant. Suitably, the plant is a tobacco plant.

In another aspect, there is provided a method for modulating the amount of nicotine in at least a part of cured plant material—such as cured leaf—comprising the steps of: (i) modulating the expression or function of an one or more of the polypeptides (or any combination thereof as described herein), suitably, wherein the polypeptide(s) is encoded by the corresponding polynucleotides described herein; (ii) harvesting plant material—such as one or more of the leaves—and curing for a period of time; (iii) measuring the level of nicotine in at least a part of the cured plant material obtained In step (ii) or during step (ii); and (iv) identifying cured plant material in which the level of nicotine therein has been modulated in comparison to a control plant.

Suitably provided herein are plants or part thereof, or cured tobacco or tobacco products wherein the level of nicotine is modulated as compared to a control plant, and the level of ammonia is the same as a control plant. Suitably provided herein are plants or part thereof, or cured tobacco or tobacco products wherein the level of nicotine is modulated as compared to a control plant, and the level of ammonia is the reduced as compared to a control plant. Suitably provided herein are plants or part thereof, or cured tobacco or tobacco products wherein the level of nicotine is reduced as compared to a control plant, and the level of ammonia is the reduced as compared to a control plant.

An increase in expression as compared to the control may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200%, 300%, 500%, 1000% or more, which includes an increase in transcriptional function or polynucleotide expression or polypeptide expression or a combination thereof.

An increase in function or activity as compared to a control may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200%, 300%, 500%, 1000% or more. A reduction in expression as compared to a control may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or 100%, which includes a reduction in transcriptional function or polynucleotide expression or polypeptide expression or a combination thereof.

A reduction in function or activity as compared to a control may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or 100%.

Polynucleotides and recombinant constructs described herein can be used to modulate the expression or function or activity of the polynucleotides or polypeptides described herein in a plant species of interest, suitably tobacco.

A number of polynucleotide based methods can be used to increase gene expression in plants and plant cells. By way of example, a construct, vector or expression vector that is compatible with the plant to be transformed can be prepared which comprises the gene of interest together with an upstream promoter that is capable of overexpressing the gene in the plant or plant cell. Exemplary promoters are described herein. Following transformation and when grown under suitable conditions, the promoter can drive expression in order to modulate the levels of this enzyme in the plant, or in a specific tissue thereof. In one exemplary embodiment, a vector carrying one or more polynucleotides described herein (or any combination thereof as described herein) is generated to overexpress the gene in a plant or plant cell. The vector carries a suitable promoter—such as the cauliflower mosaic virus CaMV 35S promoter—upstream of the transgene driving its constitutive expression in all tissues of the plant. The vector also carries an antibiotic resistance gene in order to confer selection of the transformed calli and cell lines.

The expression of sequences from promoters can be enhanced by including expression control sequences, including enhancers, chromatin activating elements, transcription factor responsive elements and the like. Such control sequences may be constitutive, and upregulate transcription in a universal manner; or they may be facultative, and upregulate transcription in response to specific signals. Signals associated with senescence and signals which are active during the curing procedure are specifically indicated.

Various embodiments are therefore directed to methods for modulating the expression level of one or more polynucleotides described herein (or any combination thereof as described herein) by integrating multiple copies of the polynucleotide into a plant genome, comprising: transforming a plant cell host with an expression vector that comprises a promoter operably-linked to one or more polynucleotides described herein. The polypeptide encoded by a recombinant polynucleotide can be a native polypeptide, or can be heterologous to the cell. One object is to provide mutant, transgenic or non-naturally occurring plants or parts thereof that exhibit modulated levels of ammonia in the plant material, for example, in cured leaves. Suitably, mutant, transgenic or non-naturally occurring plants or parts thereof that exhibit modulated levels of ammonia as compared to a control plant. Suitably, the mutant, transgenic or non-naturally occurring plants or parts thereof have substantially the same visual appearance as the control plant.

Accordingly, there is described herein mutant, transgenic or non-naturally occurring plants or parts thereof or plant cells that have modulated levels of ammonia compared to control cells or control plants. The mutant, transgenic or non-naturally occurring plants or plant cells have been modified to modulate the synthesis or function of one or more of the polypeptides described herein by modulating the expression of one or more of the corresponding polynucleotides described herein. Suitably, the modulated levels of ammonia are observed in at least cured leaves. In certain embodiments, the level of ammonia in the plant—suitably cured leaves or cured tobacco—may be modulated. In certain embodiments, the level of ammonia in the plant—suitably cured leaves or cured tobacco—may be reduced. Suitably, the modulated levels of ammonia may be observed in the green leaves. However, the ammonia level in green leaves is generally extremely low and the moderation of ammonia levels is more keenly observed in cured plant material.

A further aspect, relates to a mutant, non-naturally occurring or transgenic plant or cell, wherein the expression or the function of one or more of the polypeptides described herein is modulated and a part of the plant (for example, suitably cured leaves or cured tobacco) have decreased levels of ammonia of at least 1%, 5%, 10%, 15%, 20%, 21%, 22%, 23%, 2%, or 25% therein as compared to a control plant in which the expression or the function of said polypeptide(s) has not been modulated. In certain embodiments, the level of nicotine in the plant—such suitably cured leaves or cured tobacco—may be modulated, for example, by at least about least 1%, 5%, 10%, 15%, 20%, 21%, 22%, 23%, 2%, or 25%.

A still further aspect, relates to a cured plant material—such as cured leaf or cured tobacco—derived or derivable from a mutant, non-naturally occurring or transgenic plant or cell, wherein expression of one or more of the polynucleotides described herein or the function of the polypeptide encoded thereby is modulated and wherein the level of ammonia is modulated by at least 1%, 5%, 10%, 15%, 20%, 21%, 22%, 23%, 2%, or 25% as compared to a control plant.

Suitably the visual appearance of said plant or part thereof (for example, leaf) is substantially the same as the control plant. Suitably, the plant is a tobacco plant.

Embodiments are also directed to compositions and methods for producing mutant, non-naturally occurring or transgenic plants or plant cells that have been modified to modulate the expression or function of the one or more of the polynucleotides or polypeptides described herein which can result in plants or plant components (for example, leaves—such cured leaves—or tobacco) or plant cells with modulated ammonia content.

The mutant, non-naturally occurring or transgenic plants obtained according to this disclosure can be similar or substantially the same in visual appearance to the corresponding control plants. In one embodiment, the leaf weight of the mutant, non-naturally occurring or transgenic plant is substantially the same as the control plant. In one embodiment, the leaf number of the mutant, non-naturally occurring or transgenic plant is substantially the same as the control plant. In one embodiment, the leaf weight and the leaf number of the mutant, non-naturally occurring or transgenic plant is substantially the same as the control plant. In one embodiment, the stalk height of the mutant, non-naturally occurring or transgenic plants is substantially the same as the control plants at, for example, one, two or three or more months after field transplant or 10, 20, 30 or 36 or more days after topping. For example, the stalk height of the mutant, non-naturally occurring or transgenic plants is not less than the stalk height of the control plants. In another embodiment, the chlorophyll content of the mutant, non-naturally occurring or transgenic plants is substantially the same as the control plants. In another embodiment, the stalk height of the mutant, non-naturally occurring or transgenic plants is substantially the same as the control plants and the chlorophyll content of the mutant, non-naturally occurring or transgenic plants is substantially the same as the control plants. In other embodiments, the size or form or number or colouration of the leaves of the mutant, non-naturally occurring or transgenic plants is substantially the same as the control plants. Suitably, the plant is a tobacco plant.

In another aspect, there is provided a method for modulating the amount of ammonia in at least a part of a plant (for example, the leaves—such as cured leaves—or in tobacco), comprising the steps of: (i) modulating the expression or function of an one or more of the polypeptides described herein (or any combination thereof as described herein), suitably, wherein the polypeptide(s) is encoded by the corresponding polynucleotides described herein; (ii) measuring the level of ammonia in at least a part (for example, the leaves—such as cured leaves—or tobacco or in smoke) of the mutant, non-naturally occurring or transgenic plant obtained in step (i); and (iii) identifying a mutant, non-naturally occurring or transgenic plant in which the level of nicotine therein has been modulated in comparison to a control plant. Suitably, the visual appearance of said mutant, non-naturally occurring or transgenic plant is substantially the same as the control plant. Suitably, the plant is a tobacco plant.

In another aspect, there is provided a method for modulating the amount of ammonia in at least a part of cured plant material—such as cured leaf—comprising the steps of: (i) modulating the expression or function of an one or more of the polypeptides (or any combination thereof as described herein), suitably, wherein the polypeptide(s) is encoded by the corresponding polynucleotides described herein; (ii) harvesting plant material—such as one or more of the leaves—and curing for a period of time; (iii) measuring the level of ammonia in at least a part of the cured plant material obtained in step (ii) or during step (ii); and (iv) identifying cured plant material in which the level of ammonia therein has been modulated in comparison to a control plant.

A plant carrying a mutant allele of one or more polynucleotides described herein (or any combination thereof as described herein) can be used in a plant breeding program to create useful lines, varieties and hybrids. In particular, the mutant allele is introgressed into the commercially important varieties described above. Thus, methods for breeding plants are provided, that comprise crossing a mutant plant, a non-naturally occurring plant or a transgenic plant as described herein with a plant comprising a different genetic identity. The method may further comprise crossing the progeny plant with another plant, and optionally repeating the crossing until a progeny with the desirable genetic traits or genetic background is obtained. One purpose served by such breeding methods is to introduce a desirable genetic trait into other varieties, breeding lines, hybrids or cultivars, particularly those that are of commercial interest. Another purpose is to facilitate stacking of genetic modifications of different genes in a single plant variety, lines, hybrids or cultivars. Intraspecific as well as interspecific matings are contemplated. The progeny plants that arise from such crosses, also referred to as breeding lines, are examples of non-naturally occurring plants of the disclosure.

In one embodiment, a method is provided for producing a non-naturally occurring plant comprising: (a) crossing a mutant or transgenic plant with a second plant to yield progeny tobacco seed; (b) growing the progeny tobacco seed, under plant growth conditions, to yield the non-naturally occurring plant. The method may further comprises: (c) crossing the previous generation of non-naturally occurring plant with itself or another plant to yield progeny tobacco seed; (d) growing the progeny tobacco seed of step (c) under plant growth conditions, to yield additional non-naturally occurring plants; and I repeating the crossing and growing steps of (c) and (d) multiple times to generate further generations of non-naturally occurring plants. The method may optionally comprises prior to step (a), a step of providing a parent plant which comprises a genetic identity that is characterized and that is not identical to the mutant or transgenic plant. In some embodiments, depending on the breeding program, the crossing and growing steps are repeated from 0 to 2 times, from 0 to 3 times, from 0 to 4 times, 0 to 5 times, from 0 to 6 times, from 0 to 7 times, from 0 to 8 times, from 0 to 9 times or from 0 to 10 times, in order to generate generations of non-naturally occurring plants. Backcrossing is an example of such a method wherein a progeny is crossed with one of its parents or another plant genetically similar to its parent, in order to obtain a progeny plant in the next generation that has a genetic identity which is closer to that of one of the parents. Techniques for plant breeding, particularly plant breeding, are well known and can be used in the methods of the disclosure. The disclosure further provides non-naturally occurring plants produced by these methods. Certain embodiments exclude the step of selecting a plant.

In some embodiments of the methods described herein, lines resulting from breeding and screening for variant genes are evaluated in the field using standard field procedures. Control genotypes including the original unmutagenized parent are included and entries are arranged in the field in a randomized complete block design or other appropriate field design. For tobacco, standard agronomic practices are used, for example, the tobacco is harvested, weighed, and sampled for chemical and other common testing before and during curing. Statistical analyses of the data are performed to confirm the similarity of the selected lines to the parental line. Cytogenetic analyses of the selected plants are optionally performed to confirm the chromosome complement and chromosome pairing relationships.

DNA fingerprinting, single nucleotide polymorphism, microsatellite markers, or similar technologies may be used in a marker-assisted selection (MAS) breeding program to transfer or breed mutant alleles of a gene into other tobaccos, as described herein. For example, a breeder can create segregating populations from hybridizations of a genotype containing a mutant allele with an agronomically desirable genotype. Plants in the F2 or backcross generations can be screened using a marker developed from a genomic sequence or a fragment thereof, using one of the techniques listed herein. Plants identified as possessing the mutant allele can be backcrossed or self-pollinated to create a second population to be screened. Depending on the expected inheritance pattern or the MAS technology used, it may be necessary to self-pollinate the selected plants before each cycle of backcrossing to aid identification of the desired individual plants. Backcrossing or other breeding procedure can be repeated until the desired phenotype of the recurrent parent is recovered.

According to the disclosure, in a breeding program, successful crosses yield F1 plants that are fertile. Selected F1 plants can be crossed with one of the parents, and the first backcross generation plants are self-pollinated to produce a population that is again screened for variant gene expression (for example, the null version of the gene). The process of backcrossing, self-pollination, and screening is repeated, for example, at least 4 times until the final screening produces a plant that is fertile and reasonably similar to the recurrent parent. This plant, if desired, is self-pollinated and the progeny are subsequently screened again to confirm that the plant exhibits variant gene expression. In some embodiments, a plant population in the F2 generation is screened for variant gene expression, for example, a plant is identified that fails to express a polypeptide due to the absence of the gene according to standard methods, for example, by using a PCR method with primers based upon the polynucleotide sequence information for the polynucleotide(s) described herein (or any combination thereof as described herein).

Hybrid tobacco varieties can be produced by preventing self-pollination of female parent plants (that is, seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), or transgenic male sterility wherein a transgene inhibits microsporogenesis and/or pollen formation, or self-incompatibility. Female parent plants containing CMS are particularly useful. In embodiments in which the female parent plants are CMS, pollen is harvested from male fertile plants and applied manually to the stigmas of CMS female parent plants, and the resulting F1 seed is harvested.

Varieties and lines described herein can be used to form single-cross tobacco F1 hybrids. In such embodiments, the plants of the parent varieties can be grown as substantially homogeneous adjoining populations to facilitate natural cross-pollination from the male parent plants to the female parent plants. The F1 seed formed on the female parent plants is selectively harvested by conventional means. One also can grow the two parent plant varieties in bulk and harvest a blend of F1 hybrid seed formed on the female parent and seed formed upon the male parent as the result of self-pollination. Alternatively, three-way crosses can be carried out wherein a single-cross F1 hybrid is used as a female parent and is crossed with a different male parent. As another alternative, double-cross hybrids can be created wherein the F1 progeny of two different single-crosses are themselves crossed.

A population of mutant, non-naturally occurring or transgenic plants can be screened or selected for those members of the population that have a desired trait or phenotype. For example, a population of progeny of a single transformation event can be screened for those plants having a desired level of expression or function of the polypeptide(s) encoded thereby. Physical and biochemical methods can be used to identify expression or activity levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme function of polypeptides and polynucleotides; and polypeptide gel electrophoresis, Western blots, immunoprecipitation, and enzyme-linked immunoassays to detect polypeptides. Other techniques such as in situ hybridization, enzyme staining, and immunostaining and enzyme assays also can be used to detect the presence or expression, function or activity of polypeptides or polynucleotides.

Mutant, non-naturally occurring or transgenic plant cells and plants are described herein comprising one or more recombinant polynucleotides, one or more polynucleotide constructs, one or more double-stranded RNAs, one or more conjugates or one or more vectors/expression vectors.

Without limitation, the plants and parts thereof described herein can be modified either before or after the expression, function or activity of the one or more polynucleotides and/or polypeptides according to the present disclosure have been modulated.

One or more of the following further genetic modifications can be present in the mutant, non-naturally occurring or transgenic plants and parts thereof.

One or more genes that are involved in the conversion of nitrogenous metabolic intermediates can be modified resulting in lower levels of at least one tobacco-specific nitrosamine (TSNA). Non-limiting examples of such genes include those encoding nicotine demethylase—such as CYP82E4, CYP82E5 and CYP82E10 as described in WO2006/091194, WO2008/070274, WO2009/064771 and WO2011/088180—and nitrate reductase, as described in WO2016046288.

One or more genes that are involved in heavy metal uptake or heavy metal transport can be modified resulting in lower heavy metal content. Non-limiting examples include genes in the family of multidrug resistance associated polypeptides, the family of cation diffusion facilitators (CDF), the family of Zrt-Irt-like polypeptides (ZIP), the family of cation exchangers (CAX), the family of copper transporters (COPT), the family of heavy-metal ATPases (for example, HMAs, as described in WO2009/074325 and WO2017/129739), the family of homologs of natural resistance-associated macrophage polypeptides (NRAMP), and other members of the family of ATP-binding cassette (ABC) transporters (for example, MRPs), as described in WO2012/028309, which participate in transport of heavy metals—such as cadmium.

Other exemplary modifications can result in plants with modulated expression or function of isopropylmalate synthase which results in a change in sucrose ester composition which can be used to alter favour profile (see WO2013029799).

Other exemplary modifications can result in plants with modulated expression or function of threonine synthase in which levels of methional can be modulated (see WO2013029800).

Other exemplary modifications can result in plants with modulated expression or function of one or more of neoxanthin synthase, lycopene beta cyclase and 9-cis-epoxycarotenoid dioxygenase to modulate beta-damascenone content to alter flavour profile (see WO2013064499).

Other exemplary modifications can result in plants with modulated expression or function of members of the CLC family of chloride channels to modulate nitrate levels therein (see WO2014096283 and WO2015197727).

Other exemplary modifications can result in plants with modulated expression or function of one or more asparagine synthetases to modulate levels of asparagine in leaf and modulated levels of acrylamide in aerosol produced upon heating or combusting the leaf (see WO2017042162).

Examples of other modifications include modulating herbicide tolerance, for example, glyphosate is an active ingredient of many broad spectrum herbicides. Glyphosate resistant transgenic plants have been developed by transferring the aroA gene (a glyphosate EPSP synthetase from Salmonella typhimurium and E. coli). Sulphonylurea resistant plants have been produced by transforming the mutant ALS (acetolactate synthetase) gene from Arabidopsis. OB polypeptide of photosystem II from mutant Amaranthus hybridus has been transferred in to plants to produce atrazine resistant transgenic plants; and bromoxynil resistant transgenic plants have been produced by incorporating the bxn gene from the bacterium Klebsiella pneumoniae.

Another exemplary modification results in plants that are resistant to insects. Bacillus thuringiensis (Bt) toxins can provide an effective way of delaying the emergence of Bt-resistant pests, as recently illustrated in broccoli where pyramided cry1Ac and cry1C Bt genes controlled diamondback moths resistant to either single polypeptide and significantly delayed the evolution of resistant insects.

Another exemplary modification results in plants that are resistant to diseases caused by pathogens (for example, viruses, bacteria, fungi). Plants expressing the Xa21 gene (resistance to bacterial blight) with plants expressing both a Bt fusion gene and a chitinase gene (resistance to yellow stem borer and tolerance to sheath) have been engineered.

Another exemplary modification results in altered reproductive capability, such as male sterility.

Another exemplary modification results in plants that are tolerant to abiotic stress (for example, drought, temperature, salinity), and tolerant transgenic plants have been produced by transferring acyl glycerol phosphate enzyme from Arabidopsis; genes coding mannitol dehydrogenase and sorbitol dehydrogenase which are involved in synthesis of mannitol and sorbitol improve drought resistance.

Another exemplary modification results in plants in which the activity of one or more endogenous glycosyltransferases—such as N-acetylglucosaminyltransferase, β (1,2)-xylosyltransferase and a (1,3)-fucosyl-transferase is modulated (see WO/2011/117249). Another exemplary modification results in plants in which the activity of one or more nicotine N-demethylases is modulated such that the levels of nornicotine and metabolites of nornicotine—that are formed during curing can be modulated (see WO2015169927).

Other exemplary modifications can result in plants with improved storage polypeptides and oils, plants with enhanced photosynthetic efficiency, plants with prolonged shelf life, plants with enhanced carbohydrate content, and plants resistant to fungi. Transgenic plants in which the expression of S-adenosyl-L-methionine (SAM) and/or cystathionine gamma-synthase (CGS) has been modulated are also contemplated.

One or more genes that are involved in the nicotine synthesis pathway can be modified resulting in plants or parts of plants that when cured, produce modulated levels of nicotine. The nicotine synthesis genes can be selected from the group consisting of: A622, BBLa, BBLb, JRE5L1, JRE5L2, MATE1, MATE 2, MPO1, MPO2, MYC2a, MYC2b, NBB1, nic1, nic2, NUP1, NUP2, PMT1, PMT2, PMT3, PMT4 and QPT or a combination of one or more thereof.

One or more genes that are involved in controlling the amount of one or more alkaloids can be modified resulting in plants or parts of plants that produce modulated levels of alkaloid. Alkaloid level controlling genes can be selected from the group consisting of; BBLa, BBLb, JRE5L1, JRE5L2, MATE1, MATE 2, MYC2a, MYC2b, nic1, nic2, NUP1 and NUP2 or a combination of two or more thereof.

One or more such traits may be introgressed into the mutant, non-naturally occurring or transgenic plants from another cultivar or may be directly transformed into it.

Various embodiments provide mutant plants, non-naturally occurring plants or transgenic plants, as well as biomass in which the expression level of one or more polynucleotides according to the present disclosure are modulated to thereby modulate the level of polypeptide(s) encoded thereby.

Parts of the plants described herein, particularly the leaf lamina and midrib of such plants, can be incorporated into or used in making various consumable products including but not limited to aerosol forming materials, aerosol forming devices, smoking articles, smokable articles, smokeless products, medicinal or cosmetic products, intravenous preparations, tablets, powders, heat-not-burn (smoke-free alternative) products and tobacco products. Examples of aerosol forming materials include tobacco compositions, tobaccos, tobacco extract, cut tobacco, cut filler, cured tobacco, expanded tobacco, homogenized tobacco, reconstituted tobacco, and pipe tobaccos. Smoking articles and smokable articles are types of aerosol forming devices. Examples of smoking articles or smokable articles include cigarettes, cigarillos, and cigars. Examples of smokeless products comprise chewing tobaccos, and snuffs. In certain aerosol forming devices, rather than combustion, a tobacco composition or another aerosol forming material is heated by one or more electrical heating elements to produce an aerosol. In another type of heated aerosol forming device, an aerosol is produced by the transfer of heat from a combustible fuel element or heat source to a physically separate aerosol forming material, which may be located within, around or downstream of the heat source. Smokeless tobacco products and various tobacco-containing aerosol forming materials may contain tobacco in any form, including as dried particles, shreds, granules, powders, or a slurry, deposited on, mixed in, surrounded by, or otherwise combined with other ingredients in any format, such as flakes, films, tabs, foams, or beads. As used herein, the term ‘smoke’ is used to describe a type of aerosol that is produced by smoking articles, such as cigarettes, or by combusting an aerosol forming material. In the smoking article or smokable article, the tobacco may be combined with a binder. Examples of binders are described herein.

In one embodiment, there is also provided cured plant material from the mutant, transgenic and non-naturally occurring plants described herein. Processes of curing green tobacco leaves are known by those having skills in the art and include without limitation air-curing, fire-curing, flue-curing and sun-curing as described herein.

In another embodiment, there is described tobacco products including tobacco-containing aerosol forming materials comprising plant material—such as leaves, suitably cured leaves—from the mutant tobacco plants, transgenic tobacco plants or non-naturally occurring tobacco plants described herein. The tobacco products described herein can be a blended tobacco product which may further comprise unmodified tobacco.

In one example, leaf material—such as lamina and midrib—can be processed according to the methods described in US20190142058A1 in which a cast sheet of homogenized tobacco material is prepared by pulping cellulose fibres with water; grinding a blend of tobacco of one or more tobacco types to tobacco particles; combining the pulped cellulose fibres with the tobacco particles and with a binder to form a slurry; homogenizing the slurry; casting the slurry to form a cast sheet of homogenized tobacco material from the slurry; discarding undesired portions of the cast sheet; and introducing the discarded undesired portions of the cast sheet into the slurry. Accordingly, leaf material—such as lamina and midrib—can be combined with a binder—such as natural pectins, such as fruit, citrus or tobacco pectins; guar gums, such as hydroxyethyl guar and hydroxypropyl guar; locust bean gums, such as hydroxyethyl and hydroxypropyl locust bean gum; alginate; starches, such as modified or derivitized starches; celluloses, such as methyl, ethyl, ethylhydroxymethyl and carboxymethyl cellulose; tamarind gum; dextran; pullalon; konjac flour; xanthan gum and the like. Thus, tobacco material can comprise the leaf material as described herein and a binder. In one embodiment, there is also provided cured plant leaf material. Processes of curing green tobacco leaves are known by those having skills in the art and include without limitation air-curing, fire-curing, flue-curing and sun-curing as described herein.

In another embodiment, there is described smoke-free alternative tobacco products, for example, heat-not-burn products, comprising plant material—such as leaves, suitably cured leaves—from the mutant tobacco plants, transgenic tobacco plants or non-naturally occurring tobacco plants described herein. These smoke-free products, in which tobacco is heated rather than combusted, offer smokers alternatives to conventional cigarettes, which may reduce harmful chemicals released from the tobacco while still delivering nicotine. When tobacco is heated but not heated enough to cause it to burn, the tobacco is allowed to release a flavourful vapour which gives the user an experience of smoking tobacco but without releasing smoke or ash.

The mutant, non-naturally occurring or transgenic plants may have other uses in, for example, agriculture. For example, mutant, non-naturally occurring or transgenic plants described herein can be used to make animal feed and human food products.

The disclosure also provides methods for producing seeds comprising cultivating the mutant plant, non-naturally occurring plant, or transgenic plant described herein, and collecting seeds from the cultivated plants. Seeds from plants described herein can be conditioned and bagged in packaging material by means known in the art to form an article of manufacture. Packaging material such as paper and cloth are well known in the art. A package of seed can have a label, for example, a tag or label secured to the packaging material, a label printed on the package that describes the nature of the seeds therein.

Compositions, methods and kits for genotyping plants for identification, selection, or breeding can comprise a means of detecting the presence of a polynucleotide (or any combination thereof as described herein) in a sample of polynucleotide. Accordingly, a composition is described comprising one or more primers for specifically amplifying at least a portion of one or more of the polynucleotides and optionally one or more probes and optionally one or more reagents for conducting the amplification or detection.

Accordingly, gene specific oligonucleotide primers or probes comprising about 10 or more contiguous polynucleotides corresponding to the polynucleotide(s) described herein are disclosed. Said primers or probes may comprise or consist of about 15, 20, 25, 30, 40, 45 or 50 more contiguous polynucleotides that hybridise (for example, specifically hybridise) to the polynucleotide(s) described herein. In some embodiments, the primers or probes may comprise or consist of about 10 to 50 contiguous nucleotides, about 10 to 40 contiguous nucleotides, about 10 to 30 contiguous nucleotides or about 15 to 30 contiguous nucleotides that may be used in sequence-dependent methods of gene identification (for example, Southern hybridization) or isolation (for example, in situ hybridization of bacterial colonies or bacteriophage plaques) or gene detection (for example, as one or more amplification primers in amplification or detection). The one or more specific primers or probes can be designed and used to amplify or detect a part or all of the polynucleotide(s). By way of specific example, two primers may be used in a PCR protocol to amplify a polynucleotide fragment. The PCR may also be performed using one primer that is derived from a polynucleotide sequence and a second primer that hybridises to the sequence upstream or downstream of the polynucleotide sequence—such as a promoter sequence, the 3′ end of the mRNA precursor or a sequence derived from a vector. Examples of thermal and isothermal techniques useful for in vitro amplification of polynucleotides are well known in the art. The sample may be or may be derived from a plant, a plant cell or plant material or a tobacco product made or derived from the plant, the plant cell or the plant material as described herein.

In a further aspect, there is also provided a method of detecting a polynucleotide(s) described herein (or any combination thereof as described herein) in a sample comprising the step of: (a) providing a sample comprising, or suspected of comprising, a polynucleotide; (b) contacting said sample with one or more primers or one or more probes for specifically detecting at least a portion of the polynucleotide(s); and (c) detecting the presence of an amplification product, wherein the presence of an amplification product is indicative of the presence of the polynucleotide(s) in the sample. In a further aspect, there is also provided the use of one or more primers or probes for specifically detecting at least a portion of the polynucleotide(s). Kits for detecting at least a portion of the polynucleotide(s) are also provided which comprise one or more primers or probes for specifically detecting at least a portion of the polynucleotide(s). The kit may comprise reagents for polynucleotide amplification—such as PCR—or reagents for probe hybridization-detection technology—such as Southern Blots, Northern Blots, in-situ hybridization, or microarray. The kit may comprise reagents for antibody binding-detection technology such as Western Blots, ELISAs, SELDI mass spectrometry or test strips. The kit may comprise reagents for DNA sequencing. The kit may comprise reagents and instructions for using the kit.

In some embodiments, a kit may comprise instructions for one or more of the methods described. The kits described may be useful for genetic identity determination, phylogenetic studies, genotyping, haplotyping, pedigree analysis or plant breeding particularly with co-dominant scoring.

The present disclosure also provides a method of genotyping a plant, a plant cell or plant material comprising a polynucleotide as described herein. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. The specific method of genotyping may employ any number of molecular marker analytic techniques including amplification fragment length polymorphisms (AFLPs). AFLPs are the product of allelic differences between amplification fragments caused by polynucleotide variability. Thus, the present disclosure further provides a means to follow segregation of one or more genes or polynucleotides as well as chromosomal sequences genetically linked to these genes or polynucleotides using such techniques as AFLP analysis.

The invention is further described in the Examples below, which are provided to describe the invention in further detail. These examples, which set forth a preferred mode presently contemplated for carrying out the invention, are intended to illustrate and not to limit the invention.

### EXAMPLES

### Example 1: Identifying and Expressing Nicotinamidase Genes in Nicotiana tabacum

### Methods

Sequence comparison identified 10 Nicotinamidases in Nicotiana tabacum. NIC1A-S(SEQ ID NO: 1), NIC1A-T (SEQ ID NO:3), NIC1B-S(SEQ ID NO:5), NIC1B-T (SEQ ID NO:7), NIC3-S (SEQ ID NO:9), NIC3-T (SEQ ID NO: 11), NIC4A-S(SEQ ID NO:13), NIC4A-T (SEQ ID NO: 15), NIC4B-S(SEQ ID NO:17) and NIC4B-T (SEQ ID NO:19). The respective polypeptides encoded are shown herein as SEQ ID NO:2, 4, 6, 8, 10, 12, 14, 16, 18 and 20.

RNA from field grown Nicotiana tabacum is analysed for these sequences.

### Results

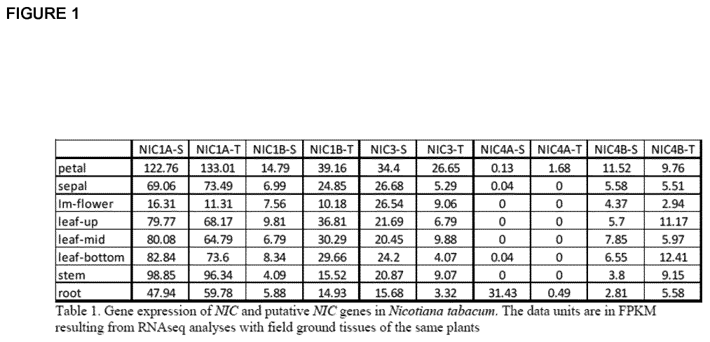

Table 1, as shown in FIG. 1, shows that the most expressed genes in leaf, sepal, stem, root and petal are NIC1A-S(SEQ ID NO:1) and NIC1A-T (SEQ ID NO:3). This suggests that both genes are essential for the recycling of NAD and energy supply in the whole plants.

NIC3-S(SEQ ID NO:9) shows greater expression than NIC3-T (SEQ ID NO:11) in all tissues, whereas the genes of the NIC4 cluster (SEQ ID NOs: 13, 15, 17 and 19) are globally poorly expressed, except NIC4A-S(SEQ ID NO: 13) in the root.

### Example 2: RNAi Silencing of Nicotinamidases

### Methods

As NIC1AS-T and NIC1B-T are remarkably expressed in all tissues (see FIG. 1), suggesting a global function in the NAD salvage pathway, the inventors focused on the two NIC3 genes (SEQ ID NO:9 and 11).

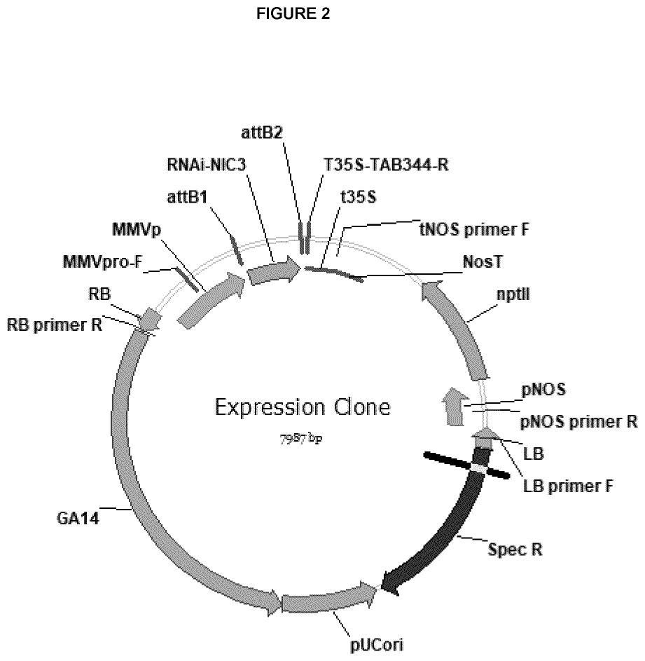

For this purpose, the silencing of NIC3 in Burley tobacco uses a classical RNAi. A specific DNA fragment within a coding sequence identical to both NIC3-S and NIC3-T (SEQ ID NO: 21) is cloned with the strong constitutive Mirabilis Mosaic Virus (MMV) promoter in a GATEWAY vector (pENTR221) as shown in FIG. 2. The NIC3 gene fragment is flanked between MMV and the 3′ nos terminator sequence of the nopaline synthase gene of Agrobacterium tumefaciens.

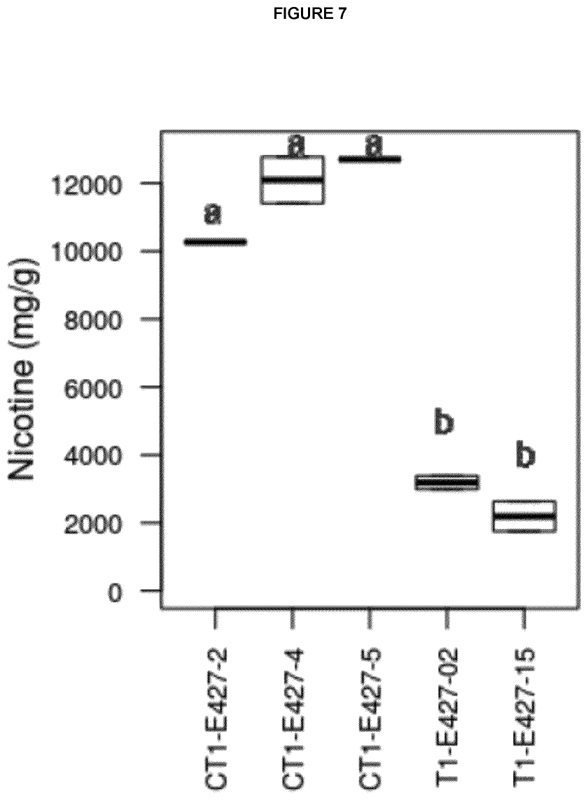

The tobacco line TN90 is transformed using standard Agrobacterium mediated transformation protocols. Two independent T1 plant lines (issued from a selection of TO plants) and wildtype plants are analysed after 48 hours of curing to determine the possible impact of the absence of tobacco nicotinamidase 3 on green leaf biomass, nicotine, content and ammonia in cured leaf. The transformed plants are referred to herein as “NIC3-RNAi” plants. (see FIG. 7)

### Results

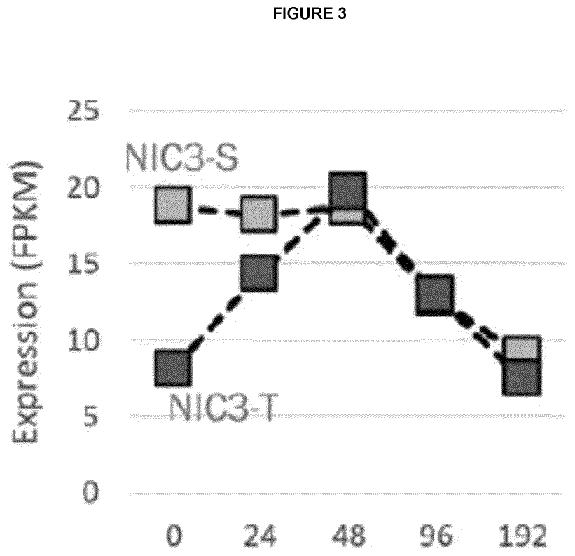

NIC3 expression is monitored by qPCR in leaves air-cured for 48 hours. This time is selected for transcription analyses, since previous data showed that NIC3-S and NIC3-T are still expressed after 48 hours air-curing, and furthermore because NIC3-T is up-regulated during the first 48 hours leaf curing (FIG. 3) thereby being easier to monitor by qPCR. The qPCR primers used are shown in SEQ ID NO:22 and SEQ ID NO:23.

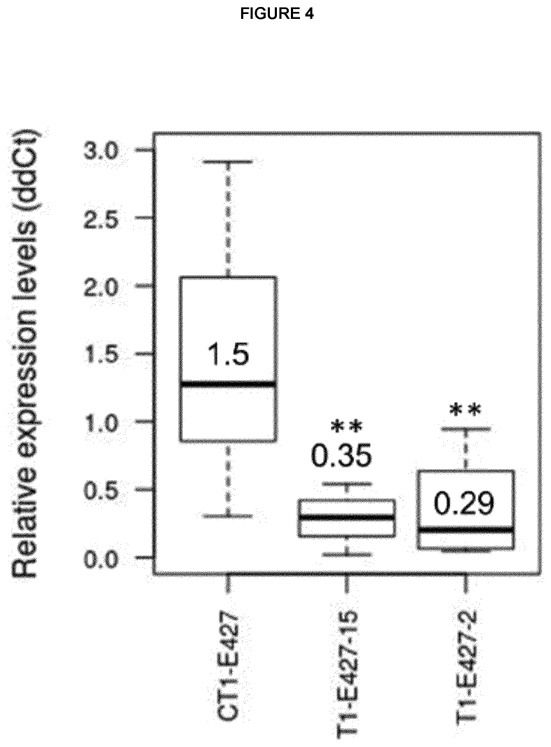

The impact on total alkaloids is then measured in the two NIC3 silenced lines compared to wildtype. As shown in FIG. 4, the reduction of the total alkaloid content compared to wildtype reached 71% in NIC3-S and 68% in NIC3-T. As total alkaloids are representative of more than 90% of the nicotine content, the rest being mainly minor alkaloids anatabine, anabasine and nornicotine, these data indicate that the two NIC3 genes encode a nicotinamidase involved in the synthesis of nicotine in roots.

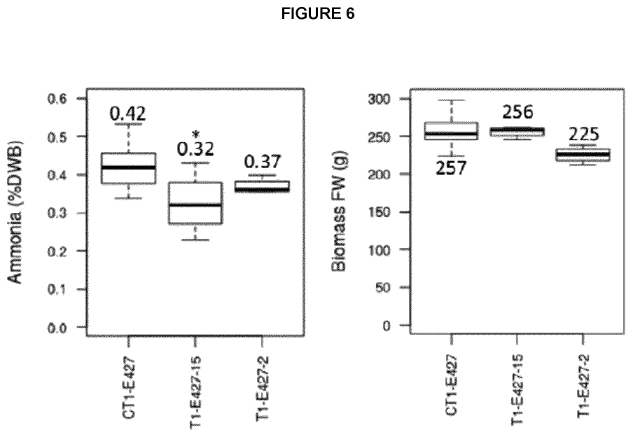

An additional observation is that there is some impact on ammonia production in cured leaves when silencing NIC3 genes, the maximum being a reduction of 24% (p<0.05) in the RNAi line T1-E427-15 when compared to wildtype (CT1-E427, FIG. 6, left). This suggests that possibly other nicotinamidase genes are playing a role in the production of ammonia (and nicotinate) during leaf curing, via the catabolism of NAD or NADP.

The inventors also show that the biomass shows only a small reduction in the RNAi silenced NIC3 plants. Only a 12% reduction of the fresh leaf biomass is observed in one independent line of NIC3-RNAi plants (FIG. 6, right). Furthermore, the plant fitness, general aspect and seed production is observed as not affected. This indicates that NIC3-S and NIC3-T (SEQ ID NO: 11 and 13) are likely not the major proteins involved in the NAD salvation pathway, but are more involved in the nicotinic acid supply for alkaloid production within the roots.

### Example 3: Selective Breeding of Nicotinamidase Mutant Lines

### Methods

An EMS-mutant library is screened to identify STOP mutations in NIC3-S(SEQ ID NO:9) and NIC3-T (SEQ ID NO:11). This screening identified two STOP mutants, one in each of NIC3-S and NIC3-T. These variant seed lines are named NIC3-S-W77* (wherein the polynucleotide including the stop codon is shown in SEQ ID NO:24 and the encoded polypeptide in SEQ ID NO: 25) and NIC3-T-W77* (wherein the polynucleotide including the stop codon is shown in SEQ ID NO:26 and the encoded polypeptide in SEQ ID NO:27) based on the position of the STOP mutation in the protein sequence. In both mutants TGG (W, tryptophan) of the wildtype sequence (SEQ ID NO: 9 to SEQ ID NO: 12) is mutated to a STOP mutation (TAG), shortening the gene product to an unfunctional 77 amino acids as shown in SEQ ID NO: 25 and SEQ ID NO: 27.

The two mutants identified are crossed to get NIC3-S-W77* and NIC3-T-W77* in the same variant line of tobacco (Nicotiana tabacum) for a breeding program to obtain seed production in a commercial tobacco background.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cellular, molecular and plant biology or related fields are intended to be within the scope of the following claims.

### Sequences

SEQ ID NO: 1 NIC1A-S coding sequence
atggtactaaaaacaatagaaattttgaagaatgaaattgccatagaggaggaatcagtgattatcactgaagat
gttaaggccggtcttgttcttgtggacatcatcaatggcttttgcaccgttggggctggaaatctggcaccaaga
gagccaaacaggcagatctctgaaatgattgatgaatcaacaaggctggctagattgttctgtgacaagaaatgg
cctattcttgcctttcttgattcacaccaccctgacaaacttgaacacccttatcctcctcactgtatcactggc
actgatgaatccaatttggttcctgcactaagatggttggagaaggaacaaaacgtaacaatcaggcgtaaagac
tgctatgatggctatattggttcgtttcaggaggatggctctaatgtatttgttgattgggtgaaaaacaacaaa
attcaaactttgttggttgtaggggcatgtacagacatttgtgtgctggatttcgtttgctctacattatcagct
aggaacccggcttcctcgaccctttggaggaagtagtagtttactctcagggatgtgccacttttgattttcctg
cctccatggcaagaaacaccaaagatatttcaccacatcctcaggagctgatgcatcatgtaggactttatatgg
cgaaggaaaggggtgctaaaatagctagagaagtctccttcaacagcttgaagaaaccatga
SEQ ID NO: 2 NIC1A-S gene product
MVLKTIEILKNEIAIEEESVIITEDVKAGLVLVDIINGFCTVGAGNLAPREPNRQISEMIDESTRLARLFCDKKW
PILAFLDSHHPDKLEHPYPPHCITGTDESNLVPALRWLEKEQNVTIRRKDCYDGYIGSFQEDGSNVFVDWVKNNK
IQTLLVVGACTDICVLDFVCSTLSARNRGFLDPLEEVVVYSQGCATFDFPASMARNKDISPHPQE
LMHHVGLYMAKERGAKIAREVSFNSLKKP
SEQ ID NO: 3 NIC1A-T coding sequence
atggtaataaaaacaatagatattttgaagaatgaaattcccatagaggaggaatcactgatcatcactgaagat
gtgaaagctggtcttgttcttgtggatatcatcaatggcttttgcactgttggagctggaaatctggcaccaaga
gagccaaacaggcagatctctgaaatgattgaggaatcaaaaaggctggctagattgttctgtgacaagaaatgg
cctattcttgcctttcttgattcacaccaacctcacaaacttgaacacccttatcctcctcactgtatcactggc
actgatgaatccaatttggttcctgcactaagatggttggagaaggaacaaaacgtaacaatcaggcgtaaagat
tgctacgatggctatattggttcttttcaggaggatggctctaatgtatttgttgattgggtgaaaaacaacaaa
attcaaactttgttggttgtaggggtatgtacagacatttgtgtgctggatttcgtttgctctacattatcagct
aagaaccgcggtttcctcaatcctttggaacaagtagtagtttactctaaaggatgtgccacttttgattttcca
gcctccatggcaagaaacagcaaagatatttcaccacatcctcaggagctgatgcatcatgtaggactttatatg
gcgaaggaaaggggtgctaaaatagctagagaagtctccttcgacagcttgaagaaaccatga
SEQ ID NO: 4 NIC1A-T gene product
MVIKTIDILKNEIPIEEESLIITEDVKAGLVLVDIINGFCTVGAGNLAPREPNRQISEMIEESKRLARLFCDKKW
PILAFLDSHQPHKLEHPYPPHCITGTDESNLVPALRWLEKEQNVTIRRKDCYDGYIGSFQEDGSNVFVDWVKNNK
IQTLLVVGVCTDICVLDFVCSTLSAKNRGFLNPLEQVVVYSKGCATFDFPASMARNSKDISPHPQE
LMHHVGLYMAKERGAKIAREVSFDSLKKP
SEQ ID NO: 5 NIC1B-S coding sequence
atgggaacggtggcgaaagaggcggcgattgatttactgaaaaatgagattccggtgaaggaagatgagcctctg
tttctctccggcgacgtcaacaccggtctcgtactcgtcgacatcgttaatggcttctgcactgtcggcgctggc
tatttggctccagtagcacctgataaacaaatttcagcaatggttgatgagtcggttaaacttgcaaaattgttt
tgtgagaagaaatggcctatttatgctcttcttgattctcatcatcctaatgtgcccgagccacctcatcctcct
cattgtatcgctggaacggatgaatctaagttggttcctgctctgcagtggttggaaagtgaaccgaatgtgaca
ctgcgatgcaaggattgcattgatgggttcgttggttcgattgagaaagatggatctaatgtctttgtagattgg
gtgaaagctaatgagattaaagctatattggttgtagggatatgcacggacatttgcctgcttgattttgtgtgt
tctgccttatctgcaaggaaccgtggatttctctcccccctggaagatgtgattgtatattcccgtggctgttct
acttttgatcttccagcacagattgctaacataaaaggagccttacctcatccacaggaattgatgcatcatata
ggcctttacatggccaaaggaagaggagcaaaagtagtttcagaggtctcctttgatacatatgcaaaagcgaca
taa
SEQ ID NO: 6 NIC1B-S gene product
MGTVAKEAAIDLLKNEIPVKEDEPLFLSGDVNTGLVLVDIVNGFCTVGAGYLAPVAPDKQISAMVDESVKLAKLF
CEKKWPIYALLDSHHPNVPEPPHPPHCIAGTDESKLVPALQWLESEPNVTLRCKDCIDGFVGSIEKDGSNVFVDW
VKANEIKAILVVGICTDICLLDFVCSALSARNRGFLSPLEDVIVYSRGCSTFDLPAQIANIKGALPHPQELMHHI
GLYMAKGRGAKVVSEVSFDTYAKAT
SEQ ID NO: 7 NIC1B-T coding sequence
atgggaacggtggcgaaagaggcggcgattgatttactgaaaaatgagattccggtggaggaagacgagcctctg
tttatctccggcgacgtcaacaccggtctcgtactcgtggacatcgttaatggcttctgcactgtcggcgctggc
aatttggctccagtggcacccgataaacaaatttcagcaatggttgatgagtcggttaaacttgcaaaaatgttt
tgtgagaagaaatggcctatttatgctcttcttgattctcatcatcctgatgtgcctgagccacctcatcctcct
cattgtatcgctggaacagatgaatctaagttggttcctgctctgcaatggttggaaagtgaaccaaatgtgaca
ctgcgatgcaaggattgcattgatgggtttcttggttcgattgagaaggatggatctaatgtcttcgtagattgg
gtgaaagctaatgagattaaagctatattggttgtagggatatgcacggacatgtgcgtgcttgattttgtgtgt
tctgccttatctgcaaggaaccgtggatttctctcccccctggaagatgtgattgtatattcccgtggctgtgct
acttttgatcttccactgcagattgctaacataaaaggagccttacctcatccacaagaattgatgcatcatata
ggcctttacatggccaaaggaagaggagcaaaagtagtttcagagatctccattgatacaactgcaagagagaca
taa
SEQ ID NO: 8 NIC1B-T gene product
MGTVAKEAAIDLLKNEIPVEEDEPLFISGDVNTGLVLVDIVNGFCTVGAGNLAPVAPDKQISAMVDESVKLAKMF
CEKKWPIYALLDSHHPDVPEPPHPPHCIAGTDESKLVPALQWLESEPNVTLRCKDCIDGFLGSIEKDGSNVFVDW
VKANEIKAILVVGICTDMCVLDFVCSALSARNRGFLSPLEDVIVYSRGCATFDLPLQIANIKGALPHPQELMHHI
GLYMAKGRGAKVVSEISIDTTARET
SEQ ID NO: 9 NIC3-S coding sequence. Underlined section = SEQ ID NO: 21
within the sequence shown
atggcttcctcatcatacaggaaatatgaaacccgaaagcgaaatccagacccgaaatctgcggttcttttagtg
atagacatacagaaccacttctactccatggccaagcctattctccccgctatcaacaccaccatcgacctctgc
cgacaggcttccgtgcctgtgatattcacgcgccaccgtcacaagtcaccggatgactacggcatgctctacgag
tggtggaacggagacgtcatcagagatggcaccattgaagctgaactcataccggagttgaaccggagagatgct
gacttggtggtagaaaagcacacttatagtgccttcagagatacaagcctagaggagaaactgttggagatgggg
ataacggaggtaatagtgagtggagtaatgaccaacttgtgttgtgagactacagccagagaggcctttgtgaga
gggttcagagtcttcttttccacagatgctactgctacttcctcagcagaattgcatgatgctacactgaagaac
ttagcttatggtttcacttatttggttgactgcaaaaggattcaagatgcgttttcaaattcctga
SEQ ID NO: 10 NIC3-S gene product
MASSSYRKYETRKRNPDPKSAVLLVIDIQNHFYSMAKPILPAINTTIDLCRQASVPVIFTRHRHKSPDDYGMLYE
WWNGDVIRDGTIEAELIPELNRRDADLVVEKHTYSAFRDTSLEEKLLEMGITEVIVSGVMTNLCCETTAREAFVR
GFRVFFSTDATATSSAELHDATLKNLAYGFTYLVDCKRIQDAFSNS
SEQ ID NO: 11 NIC3-T coding sequence. Underlined section = SEQ ID NO: 21
within the sequence shown
atggcttcctcatcatacaggaaatatgaaacccgaaagcgaaatccagacccgaaatctgcggttcttttagtg
atagatatgcagaactacttctactccatggccaagcctattctccccgctatcaacactaccatcgacctctgc
cgacaggcttccgtgcctgtgatattcacgcgccaccgtcacaagtcaccggatgactacggcatgctctacgag
tggtggaacggagacatcattaaagacggcaccgttgaggctgaactcataccggagttggaccgaagagaaagc
gacttggtggtggaaaaacacacttacaatacaaacctagaggagaaactgttggagatggggaaaaaggaggta
atagtgagtggagtaatgaccaacctgtgttgtgagacaacagccagagaggcatttgttagaggattcagagtc
ttcttttcgacagatgctactgctacttcctcagcagaattgcatgatgctacactgaagaacttagcatatggt
ttcacttatttggttgactgcaaaaggattcaatctgcgttttcaaattcctga
SEQ ID NO: 12 NIC3-T gene product
MASSSYRKYETRKRNPDPKSAVLLVIDMQNYFYSMAKPILPAINTTIDLCRQASVPVIFTRHRHKSPDDYGMLYE
WWNGDIIKDGTVEAELIPELDRRESDLVVEKHTYNTNLEEKLLEMGKKEVIVSGVMTNLCCETTAREAFVRGERV
FFSTDATATSSAELHDATLKNLAYGFTYLVDCKRIQSAFSNS
SEQ ID NO: 13 NIC4A-S coding sequence
atggcctcggaattaaatgttggcaaatggaagaacgtagctctccttgtcatagacatgcagaaagacttcata
tttggccctatgcaagtaaaaggaggtcaagctatcgtccctaacgttatcaaagctgttgaggttgcaagaaac
tgtggcattcacatcgtttgggttgtacgtgagaatgatccattagggagagatgttgaattatttcgtcgacac
tttaaaccaaagctggcatcaaagggtagtgttggggctgaactagttgatgggctcgttatggacgtagaagag
gattacaagctggtcaaaacacgtttcagcgcatttcttaacactcaccttcactcatatcttcagaccaatggc
attactgatttggtcttcactggtgtccaaacaccaaattgcatacggcaaactgtatttgatgcagtatcattg
gactataaacaagtgacagttattactgacgccacagctgctgctacacctgatatacacattgcaaacatactt
gacatgaaaaatattggagtagcaacccctacattggaagaatggtgccaatctacatag
SEQ ID NO: 14 NIC4A-S gene product
MASELNVGKWKNVALLVIDMQKDFIFGPMQVKGGQAIVPNVIKAVEVARNCGIHIVWVVRENDPLGRDVELFRRH
FKPKLASKGSVGAELVDGLVMDVEEDYKLVKTRFSAFLNTHLHSYLQTNGITDLVFTGVQTPNCIRQTVEDAVSL
DYKQVTVITDATAAATPDIHIANILDMKNIGVATPTLEEWCQST
SEQ ID NO: 15 NIC4A-T coding sequence
atggccacggaatatgtaggcaaatggaagaacgtagctctccttgtcatagacatgcagatagacttcatattt
ggccctatgcaagtaaaaggaggtcaagctatcgtccctaacgttatcaaagctgttgaggttgcgagaaactgt
ggcattcacatcgtttgggttgtacgggagaatgatccattagggagagatgttgaattatttcgtcggcacctt
aaaccaaagctggcatcaaagggtagtgtaggggctgaactaattgatgggctcgttatggacgtagaaaaggat
tacaagctggtcaaaacacgtttcagtgcatttcttaacacgcaccttcactcatattttcagaccaatggcatt
actaatctggtcttcaccggtgtccaaacaccaaattgcatacggcaaactgtgtttgatgcagtatcattggac
tataaacaagtgacagttattactgacgccacagctgctgctacacctgatatacacatt
SEQ ID NO: 16 NIC4A-T gene product
MATEYVGKWKNVALLVIDMQIDFIFGPMQVKGGQAIVPNVIKAVEVARNCGIHIVWVVRENDPLGRDVELFRRHL
KPKLASKGSVGAELIDGLVMDVEKDYKLVKTRFSAFLNTHLHSYFQTNGITNLVFTGVQTPNCIRQTVEDAVSLD
YKQVTVITDATAAATPDIHI
SEQ ID NO: 17 NIC4B-S coding sequence
atggcctcagaaaatggttgtaaatggaacaaaaccgctcttcttgtcatagacatgcagaaagattttgtacta
ccaggtgctcctatgctagttaaaggaggcgaagctattgttcctaacgttatcaagactgttgaggttgcaaga
aaccgttgcatccccattatttgggttgtccgtgagcatgatccattaggtagagatgttgaattgtttcgtcgg
catttatatggtgatgggaaaccaaaacctacatcaaagggtagtgtcggggcagaattggttgatgggcttgtt
attcaggaagatgattacaaattggtgaaaacccggttcagtgcattttttaacacgaaccttcattcgtatctt
cagggcattggcattaccaacttggtcattattggtgttcaaactccaaattgcatacggcagactgtctttgat
gctgtagcattggactatgaacgtgttacggtcattatagatgccacagctgctgctactcctgatatacatatc
gcaaacataattgacatgaaaaatgtaggggtggtgactcctacgttagaagaatggtgccagtctatggagcaa
tag
SEQ ID NO: 18 NIC4B-S gene product
MASENGCKWNKTALLVIDMQKDFVLPGAPMLVKGGEAIVPNVIKTVEVARNRCIPIIWVVREHDPLGRDVELFRR
HLYGDGKPKPTSKGSVGAELVDGLVIQEDDYKLVKTRFSAFFNTNLHSYLQGIGITNLVIIGVQTPNCIRQTVED
AVALDYERVTVIIDATAAATPDIHIANIIDMKNVGVVTPTLEEWCQSMEQ
SEQ ID NO: 19 NIC4B-T coding sequence
atggcctcagaaaatggttgtaaatggaacaaaactgctcttcttgtcatagacatgcagaaagattttgtacta
ccaggtgctcctatgctagttaaaggaggcgaagctattgttcctaacgttatcaagaccgttgaggttacaaga
agccgcggcatccccattatttgggttgtccgtgagcatgatcaattaggtagagatgttgaattgtttcgtcgg
catttatatggtgatggaaaaccaaaaccaacatcaaagggtagtgtgggggcagagctagttgatgggcttgtt
attgaggaagatgattacaaattggtgaaaacacggttcagtgcattttttaacacgaaccttcattcgtatctt
cagggcattggcattaccaacttggtcattattggtgttcaaactccaaactgcatacggcagactgtctttgat
gctgtagcattggactatgaacgtgttacggtcattatagatgccacagctgctgctactcctgatatacatatc
gcgaacatatttgacatgaaaaatgtgggggtggcaactcctacgttagaagaatggtgccggtctaaggagcaa
tacaagtgctag
SEQ ID NO: 20 NIC4B-T gene product
MASENGCKWNKTALLVIDMQKDFVLPGAPMLVKGGEAIVPNVIKTVEVTRSRGIPIIWVVREHDQLGRDVELFRR
HLYGDGKPKPTSKGSVGAELVDGLVIEEDDYKLVKTRFSAFFNTNLHSYLQGIGITNLVIIGVQTPNCIRQTVED
AVALDYERVTVIIDATAAATPDIHIANIFDMKNVGVATPTLEEWCRSKEQYKC
SEQ ID NO: 21 NIC3 sequence insert for RNAi plasmid construct
acagatgctactgctacttcctcagcagaattgcatgatgctacactgaagaacttagc
SEQ ID NO: 22 NIC3 specific qPCR forward primer
ttcctcatcatacaggaaatatgaaa
SEQ ID NO: 23 NIC3 specific qPCR reverse primer
actcgtagagcatgccgtagtc
SEQ ID NO: 24 NIC3-S-W77* coding sequence
atggcttcctcatcatacaggaaatatgaaacccgaaagcgaaatccagacccgaaatctgcggttcttttagtg
atagacatacagaaccacttctactccatggccaagcctattctccccgctatcaacaccaccatcgacctctgc
cgacaggcttccgtgcctgtgatattcacgcgccaccgtcacaagtcaccggatgactacggcatgctctacgag
tggtag
SEQ ID NO: 25 NIC3-S-W77* gene product
MASSSYRKYETRKRNPDPKSAVLLVIDIQNHFYSMAKPILPAINTTIDLCRQASVPVIFTRHRHKSPDDYGM
LYEW*
SEQ ID NO: 26 NIC3-T-W77* coding sequence
atggcttcctcatcatacaggaaatatgaaacccgaaagcgaaatccagacccgaaatctgcggttcttttagtg
atagatatgcagaactacttctactccatggccaagcctattctccccgctatcaacactaccatcgacctctgc
cgacaggcttccgtgcctgtgatattcacgcgccaccgtcacaagtcaccggatgactacggcatgctctacgag
tggtag
SEQ ID NO: 27 NIC3-T-W77* gene product
MASSSYRKYETRKRNPDPKSAVLLVIDMQNYFYSMAKPILPAINTTIDLCRQASVPVIFTRHRHKSPDDYGMLYE
W*

1. A plant cell having modulated expression or activity of a nicotinamidase enzyme, said plant cell comprising:
(a) a polynucleotide sequence comprising, consisting or consisting essentially of a sequence encoding the nicotinamidase enzyme; or
(b) a polypeptide encoded by the polynucleotide set forth in (a);
wherein said plant cell comprises at least one modification which modulates the expression or activity of the polynucleotide or the polypeptide as compared to a control plant cell in which the expression or activity of the polynucleotide or polypeptide has not been modified,
wherein said polynucleotide (a) comprises:
(i) a polynucleotide comprising, consisting or consisting essentially of a sequence having at least 70% sequence identity to, SEQ ID NO:9, SEQ ID NO:11, SEQ ID NO:24 or SEQ ID NO: 26; and/or
(ii) a construct, vector or expression vector comprising the isolated polynucleotide set forth in (i) or set forth in SEQ ID NO:21.

2. The plant cell according to claim 1, wherein said polypeptide (b) comprises:
a polypeptide encoded by the polynucleotide set forth in claim 1; and/or
a polypeptide comprising, consisting or consisting essentially of a sequence having at least 90% sequence identity to SEQ ID NO:10, SEQ ID NO:12, SEQ ID NO:25 or SEQ ID NO:27.

3. The plant cell according to claim 1, wherein said plant cell comprises a polynucleotide comprising, consisting or consisting essentially of a sequence having at least 80% sequence identity to SEQ ID NOs: 9, 11, 24 or 26 suitably, wherein the plant cell comprises a polynucleotide comprising, consisting or consisting essentially of a sequence having at least 85% sequence identity to SEQ ID NOs: 9, 11, 24 or 26; or
wherein said plant cell comprises a polypeptide comprising, consisting or consisting essentially of a sequence having at least 95% sequence identity to SEQ ID NOs: 10, 12, 25 or 27 or at least 94% sequence identity to or SEQ ID NOs: 10, 12, 25 or 27 suitably, wherein the plant cell comprises a polypeptide comprising, consisting or consisting essentially of a sequence having at least 93% sequence identity to SEQ ID NOs: 10, 12, 25 or 27.

4. The plant cell according to claim 1, wherein the at least one modification is a modification of the plant cell's genome, or a modification of the construct, vector or expression vector, or a transgenic modification; suitably,
wherein the modification of the plant cell's genome, or the modification of the construct, vector, or expression vector is a mutation or edit,
most suitably wherein the modification of the plant cell's genome, or the modification of the construct, vector, or expression vector is a truncation.

5. The plant cell according to claim 1, wherein the modification decreases the expression or activity of the polynucleotide or the polypeptide as compared to the control plant cell; suitably,
wherein the plant cell comprises an interference polynucleotide comprising a sequence that is at least 80% complementary to at least 50 nucleotides of an RNA transcribed from the polynucleotide of claim 1(i).

6. The plant cell according to claim 1, wherein the modulated expression or activity of the polynucleotide or the polypeptide modulates the level of nicotine in cured or dried leaf derived from the plant cell as compared to the level of nicotine in cured or dried leaf derived from a control plant, suitably
wherein the level of nicotine in cured or dried leaf derived from the plant cell is decreased as compared to the level of nicotine in cured or dried leaf derived from a control plant, and/or
wherein the modulated expression or activity of the polynucleotide or the polypeptide modulates the level of ammonia in cured or dried leaf derived from the plant cell as compared to the level of ammonia in cured or dried leaf derived from a control plant, suitably
wherein the level of ammonia in cured or dried leaf derived from the plant cell is decreased as compared to the level of ammonia in cured or dried leaf derived from a control plant.

7. A plant or part thereof comprising the plant cell according to claim 1; suitably,
wherein the amount of nicotine is modified in at least a part of the plant as compared to a control plant or part thereof;
suitably wherein the amount of nicotine is reduced in at least a part of the plant as compared to a control plant or part thereof;
suitably wherein the amount of nicotine is reduced in at least the leaves of the plant as compared to the leaves of a control plant or part thereof.

8. Plant material, cured plant material, or homogenized plant material, derived from the plant or part thereof of claim 7, suitably
wherein the cured plant material is air-cured or sun-cured or flue-cured plant material; suitably,
wherein the plant material, cured plant material, or homogenized plant material comprises biomass, seed, stem, flowers, or leaves from the plant or part thereof of claim 7.

9. A tobacco product comprising the plant cell of claim 1, suitably wherein said tobacco product comprises reduced nicotine content, suitably wherein said tobacco product comprises reduced nicotine content compared to a control tobacco product, suitably wherein said tobacco product comprises reduced ammonia content, suitably wherein said tobacco product comprises reduced ammonia content compared to a control tobacco product, suitability said tobacco product is a smoke-free alternative tobacco product.

10. A method for producing the plant of claim 7, comprising the steps of:
(a) providing a plant cell of any comprising a polynucleotide comprising, consisting or consisting essentially of a polynucleotide comprising, consisting or consisting essentially of a sequence having at least 80% sequence identity to SEQ ID NOs: 9, 11, 13, 24 or 26;
(b) modifying the plant cell to modulate the expression of said polynucleotide as compared to a control plant cell; and
(c) propagating the plant cell into a plant,
suitably wherein step (c) comprises cultivating the plant from a cutting or seedling comprising the plant cell,
optionally wherein the step of modifying the plant cell comprises introducing a stop codon to the polynucleotide comprising, consisting or consisting essentially of a sequence having at least 80% sequence identity to SEQ ID NOs: 9, 11, 24 or 26,
suitably wherein introduction of said stop codon causes truncation of polypeptide encoded by the polynucleotide comprising, consisting or consisting essentially of a sequence having at least 80% sequence identity to SEQ ID NOs: 9, 11, 24 or 26.

11. The method according to claim 10, wherein the step of modifying the plant cell comprises modifying the genome of the cell by genome editing or genome engineering; suitably,
wherein the genome editing or genome engineering is selected from CRISPR/Cas technology, zinc finger nuclease-mediated mutagenesis, chemical or radiation mutagenesis, homologous recombination, oligonucleotide-directed mutagenesis and meganuclease-mediated mutagenesis.

12. The method according to claim 10, wherein the step of modifying the plant cell comprises transfecting the cell with a construct comprising a polynucleotide comprising, consisting, or consisting essentially of a sequence having at least 80% sequence identity to SEQ ID NO: 21 operably linked to a promoter; and/or
wherein the step of modifying the plant cell comprises introducing an interference polynucleotide comprising a sequence that is at least 80% complementary to an RNA transcribed from the polynucleotide of claim 1(i) into the cell; suitably,
wherein the plant cell is transfected with a construct expressing an interference polynucleotide comprising a sequence that is at least 80% complementary to at least 50 nucleotides of an RNA transcribed from the polynucleotide of claim 1(i).

13. A tobacco product comprising a plant cell according to claim 1, said tobacco product comprising a reduced nicotine level, suitably wherein said tobacco product comprises a reduced nicotine level compared to a control tobacco product, suitably wherein said tobacco product comprises a nicotine level reduced by at least 10%, 15%, 20%, 25%, 30%, 25%, 40%, 45%, 50%, 55%, 60%, 65%, 70% or 75% compared to a control tobacco product,
optionally wherein said tobacco product further comprises reduced ammonia level, suitably wherein said tobacco product comprises reduced ammonia level compared to a control tobacco product. suitably wherein said tobacco product comprises an ammonia level reduced by at least 10%, 15%, 20%, 25%, 30%, 25%, 40%, 45%, 50%, 55%, 60%, 65%, 70% or 75% compared to a control tobacco product.

14. A method for producing cured plant material with an altered amount of nicotine derived therefrom or an altered amount of nicotine as compared to control plant material, comprising the steps of:
(a) providing a plant or part thereof according to claim 7;
(b) optionally harvesting the plant material therefrom; and
(c) curing the plant material; suitably,
wherein the plant material comprises cured leaves, cured stems or cured flowers, or a mixture thereof; and/or
wherein the curing method is selected from the group consisting of air curing, fire curing, smoke curing, and flue curing.
